(19) United States
(12) Patent Application Publication
(10) Pub. No.: US 2017/0152486 A1
(43) Pub. Date: Jun. 1, 2017

(54) METHOD FOR CULTURE OF HUMAN BLADDER CELL LINES AND ORGANOIDS AND USES THEREOF

(71) Applicant: THE TRUSTEES OF COLUMBIA UNIVERSITY IN THE CITY OF NEW YORK, New York, NY (US)

(72) Inventors: Michael M. SHEN, New York, NY (US); Lamont Jordan BARLOW, New York, NY (US); Chee Wai CHUA, New York, NY (US)

(21) Appl. No.: 15/288,871

(22) Filed: Oct. 7, 2016

Related U.S. Application Data
(63) Continuation-in-part of application No. PCT/US15/19013, filed on Mar. 5, 2015.
(60) Provisional application No. 61/976,247, filed on Apr. 7, 2014.

(51) Int. Cl. C12N 5/071; C12N 5/09; G01N 33/50

(52) U.S. Cl. CPC C12N 5/0685; G01N 33/5044; G01N 33/5011; C12N 5/0693; C12N 2501/727; C12N 2506/30; C12N 2501/11; C12N 2509/00; C12N 2509/10; C12N 2533/90

(57) ABSTRACT

The invention discloses a methodology for the culture of bladder cell lines and organoids from human bladder, both non-cancerous as well as cancer tissue.

This application is a continuation-in-part of International Application No. PCT/US2015/019013, filed Mar. 5, 2015 which claims the benefit of and priority to U.S. provisional patent application Ser. No. 61/976,247 filed Apr. 7, 2014, the disclosure of all of which is hereby incorporated by reference in its entirety for all purposes.

### GOVERNMENT SUPPORT

This invention was made with government support under Grant No. P01 CA154293 awarded by the National Institute of Health/National Cancer Institute. The government has certain rights in the invention.

All patents, patent applications and publications, and other literature references cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

There were over 72,000 new cases of bladder cancer in America in 2013, and 15,000 people died from the disease. The majority of patients are diagnosed with non-muscle-invasive bladder cancer (NMIBC), or cancer which remains in the superficial layers of the bladder. The standard treatment for NMIBC is to remove the tumor endoscopically through a procedure called a “transurethral resection of bladder tumor” (TURBT). After TURBT, many patients are also given either immunotherapy or chemotherapy directly into the bladder; this treatment, referred to as “intravesical therapy,” can reduce the risk of recurrence and progression. However, many patients will not respond to intravesical therapy and require partial or complete surgical removal of the bladder (“cystectomy”). Unfortunately, there are currently no established methods to predict whether or not an individual patient will have a response to any specific intravesical agent. Patients who do not respond are at risk of disease progression the longer they keep their bladder. It would be ideal to distinguish patients most likely to respond to various intravesical agents from others who are unlikely to respond to any agent and should undergo immediate bladder removal.

Compared to other common malignancies, there are few available intravesical agents; this is largely due to the fact that it is difficult to conduct clinical trials due to slow study accrual and lack of funding. There is also a limited availability of preclinical bladder cancer models to test drug activity. This invention relates to the culture of bladder cell lines and organoids from human bladder tissue.

### SUMMARY OF THE INVENTION

The present invention provides methods for culturing bladder cell lines or organoids from bladder tissue.

In one aspect, the invention provides a method for culturing a bladder cell line, the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture. In one embodiment, the bladder tissue is non-cancerous. In another embodiment, the bladder tissue is cancerous. In another embodiment, the bladder tissue is obtained from a bladder tumor. In a further embodiment, the subject is a human. In another embodiment, the bladder tissue is obtained from an endoscopic biopsy, an endoscopic resection, or a cystectomy sample. In a further embodiment, the bladder cell line displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% Matrigel. In another embodiment, the culture medium comprises 5% heat-inactivated charcoal stripped FBS. In another embodiment, the ROCK inhibitor is Y-27632. In another embodiment, the culture medium comprises 10 μM of Y-27632. In one embodiment, the cells in the bladder cell line grow as adherent cells in two-dimensional culture. In another embodiment, a single cell suspension is obtained by the dissociating of (b). In a further embodiment, the single cell suspension contains epithelial and stromal cells. In another embodiment, (b) comprises dissociating the sample of bladder tissue with collagenase, hyaluronidase, dispase, or a combination thereof. In another embodiment, the isolating of (c) is by immunomagnetic cell separation. In a further embodiment, the immunomagnetic cell separation uses an antibody against Epithelial Cell Adhesion Molecule (EpCAM). In one embodiment, the method further comprises: (e) serially passaging the bladder cell line colonies. In another embodiment, the adherent cell culture support is a tissue culture plate that enhances or maximizes attachment of the cells to the surface of the support. In another embodiment, the adherent cell culture support is a Primaria™ surface modified cell culture plate. In another embodiment, the method has at least 80% efficiency. In another embodiment, the method has at least 85% efficiency. In another embodiment, the method has at least 89% efficiency. In another embodiment, the method has at least 90% efficiency.

In one aspect, the invention provides a method for culturing a bladder organoid, the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture. In one embodiment, the bladder tissue is non-cancerous. In another embodiment, the bladder tissue is cancerous. In another embodiment, the bladder tissue is obtained from a bladder tumor. In a further embodiment, the subject is a human. In another embodiment, the bladder tissue is obtained from an endoscopic biopsy, an endoscopic resection, or a cystectomy sample. In a further embodiment, the bladder organoid displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% Matrigel. In another embodiment, the culture medium comprises 5% heat-inactivated charcoal stripped FBS. In another embodiment, the ROCK inhibitor is Y-27632. In another embodiment, the culture medium comprises 10 μM of Y-27632. In one embodiment, a bladder cell line is obtained from the organoids. In one embodiment, the cells in the bladder cell line grow as adherent cells in two-dimensional culture. In another embodiment, a single cell suspension is obtained by the dissociating of (b). In a further embodiment, the single cell suspension contains epithelial and stromal cells. In another embodiment, (b) comprises dissociating the sample of bladder tissue with collagenase, hyaluronidase, dispase, or a combination thereof. In another embodiment, the isolating of (c) is by immunomagnetic cell separation. In a further embodiment, the immunomagnetic cell separation uses an antibody against Epithelial Cell Adhesion Molecule (EpCAM). In one embodiment, the method further comprises: (e) serially passaging the bladder cell line colonies. In another embodiment, low attachment cell culture support is a tissue culture plate that minimizes or prevents attachment of the cells to the surface of the support. In another embodiment, the low attachment cell culture support is a Ultra-Low Attachment 96 well plate. In another embodiment, the method has at least 80% efficiency. In another embodiment, the method has at least 85% efficiency. In another embodiment, the method has at least 89% efficiency. In another embodiment, the method has at least 90% efficiency.

In one aspect, the invention provides a method for culturing a bladder organoid, the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids. In one embodiment, the bladder tissue is non-cancerous. In another embodiment, the bladder tissue is cancerous. In another embodiment, the bladder tissue is obtained from a bladder tumor. In one embodiment, the subject is a human. In another embodiment, the bladder tissue is obtained from an endoscopic biopsy, an endoscopic resection, or a cystectomy sample. In a further embodiment, the bladder organoid displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% heat-inactivated charcoal stripped FBS. In one embodiment, a bladder cell line is obtained from the organoids. In one embodiment, the cells in the bladder cell line grow as adherent cells in two-dimensional culture. In another embodiment, a single cell suspension is obtained by the dissociating of (b). In another embodiment, cell clusters are obtained by the dissociating of (b). In a further embodiment, the single cell suspension contains epithelial and stromal cells. In a further embodiment, the cell clusters contain epithelial and stromal cells. In another embodiment, (b) comprises dissociating the sample of bladder tissue with collagenase, hyaluronidase, or a combination thereof. In another embodiment, the dissociating further comprises dissociating the sample with TrypLE™ or trypsin. In one embodiment, the method further comprises: (f) serially passaging the bladder cell line colonies. In another embodiment, the cell culture support is a 6-well tissue culture plate. In another embodiment, the cell culture support is surface modified before the plating by rinsing Matrigel solution over the support surface and incubating the cell culture support at 37° C. for at least 30 minutes. In another embodiment, the method has at least 80% efficiency. In another embodiment, the method has at least 85% efficiency. In another embodiment, the method has at least 89% efficiency. In another embodiment, the method has at least 90% efficiency.

In one aspect, the invention provides a bladder cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture. In one embodiment, the subject is a human. In another embodiment, the cell line is preserved in a tissue bank.

In one aspect, the invention provides a bladder cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids. In one embodiment, the subject is a human. In another embodiment, the cell line is preserved in a tissue bank.

In one aspect, the invention provides a bladder cell line, wherein the cell line is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids and wherein a bladder cell line is obtained from the organoids. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids and wherein a bladder cell line is obtained from the organoids. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder organoid, wherein the organoid is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture. In one embodiment, the subject is a human. In another embodiment, the cell line is preserved in a tissue bank.

In one aspect, the invention provides a bladder tumor organoid, wherein the organoid is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture. In one embodiment, the bladder organoid displays the transformed phenotype of cancerous bladder tissue. In one embodiment, the subject is a human. In another embodiment, the cell line is preserved in a tissue bank.

In one aspect, the invention provides a bladder organoid, wherein the organoid is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids. In one embodiment, the subject is a human. In another embodiment, the cell line is preserved in a tissue bank.

In one aspect, the invention provides a bladder tumor organoid, wherein the organoid is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids. In one embodiment, the bladder organoid displays the transformed phenotype of cancerous bladder tissue. In one embodiment, the subject is a human. In another embodiment, the cell line is preserved in a tissue bank.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on an adherent cell culture support; and (v) culturing the dissociated bladder epithelial cells in a culture medium comprising hepatocyte medium, FBS, Matrigel, and ROCK inhibitor; wherein the dissociated bladder epithelial cells form bladder cell line colonies in culture; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on an adherent cell culture support; and (v) culturing the dissociated bladder epithelial cells in a culture medium comprising hepatocyte medium, FBS, Matrigel, and ROCK inhibitor; wherein the dissociated bladder epithelial cells form bladder cell line colonies in culture; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder organoid with a test compound, wherein the organoid is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor organoid with a test compound, wherein the organoid is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder organoid with a test compound, wherein the organoid is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor organoid with a test compound, wherein the organoid is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; plating the isolated dissociated bladder epithelial cells of (c) on an adherent cell culture support; (e) culturing the dissociated bladder epithelial cells in a culture medium comprising hepatocyte medium, FBS, Matrigel, and ROCK inhibitor, wherein the dissociated bladder epithelial cells form bladder cell line colonies in culture; (e) contacting the bladder cell line with a test compound; and (f) determining whether growth of the bladder cell line is inhibited in the presence of the test compound, as compared to growth of the bladder cell line in the absence of the test compound, wherein the test compound is administered to the subject if growth of the bladder cell line is inhibited in the presence of the test compound. In one embodiment, the test compound is an intravesical agent. In another embodiment, the test compound is an antineoplastic agent. In a further embodiment, the test compound is a chemotherapy agent. In another embodiment, the growth of the bladder cell line of (f) is measured using a MTT assay.

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; (d) plating the isolated dissociated bladder epithelial cells of (c) on an adherent cell culture support; (e) culturing the dissociated bladder epithelial cells in a culture medium comprising hepatocyte medium, FBS, Matrigel, and ROCK inhibitor, wherein the dissociated bladder epithelial cells form bladder cell line colonies in culture; (e) contacting the bladder cell line with a test compound; and (f) determining whether growth of the bladder cell line is inhibited in the presence of the test compound, as compared to growth of the bladder cell line in the absence of the test compound, wherein a cystectomy is performed on the subject if growth of the bladder cell line is not inhibited in the presence of the test compound. In one embodiment, the test compound is an intravesical agent. In another embodiment, the test compound is an antineoplastic agent. In a further embodiment, the test compound is a chemotherapy agent. In another embodiment, the growth of the bladder cell line of (f) is measured using a MTT assay.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; (e) incubating the culture of (d) wherein the dissociated bladder tissue forms organoids; (f) contacting the bladder organoid with a test compound; and determining whether growth of the bladder organoid is inhibited in the presence of the test compound, as compared to growth of the bladder organoid in the absence of the test compound, wherein the test compound is administered to the subject if growth of the bladder organoid is inhibited in the presence of the test compound. In one embodiment, the test compound is an intravesical agent. In another embodiment, the test compound is an antineoplastic agent. In a further embodiment, the test compound is a chemotherapy agent. In another embodiment, the growth of the bladder cell line of (f) is measured using a MTT assay.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; (e) incubating the culture of (d) wherein the dissociated bladder tissue forms organoids; (f) contacting the bladder organoid with a test compound; and determining whether growth of the bladder organoid is inhibited in the presence of the test compound, as compared to growth of the bladder organoid in the absence of the test compound, wherein a cystectomy is performed on the subject if growth of the bladder cell line is not inhibited in the presence of the test compound. In one embodiment, the test compound is an intravesical agent. In another embodiment, the test compound is an antineoplastic agent. In a further embodiment, the test compound is a chemotherapy agent. In another embodiment, the growth of the bladder cell line of (f) is measured using a MTT assay.

### BRIEF DESCRIPTION OF THE FIGURES

The patent or application file contains at least one color drawing executed in color. Copies of this patent or patent application publication with color drawing(s) will be provided by the United States Patent and Trademark Office upon request and payment of the necessary fee.

FIG. 1 shows a schematic of the method for establishing patient-specific bladder cancer cell cultures for drug sensitivity testing.

FIGS. 2A-2F shows patient-derived bladder cancer cell lines in culture. FIG. 2A: Single cells are seen on day 1 of adherent culture. FIG. 2B: Small colonies are seen by day 6. FIG. 2C: Large colonies with moderate confluence seen on day 12. FIG. 2D: Colonies are seen on day 5 after two passages. FIGS. 2E-F: Spherical “organoids” form when cells are grown in 3-dimensional floating culture.

FIGS. 3A-3O shows immunohistochemical analysis of patient-derived cell lines. FIGS. 3A-E: Histological analysis of parental tumor tissue from Line #7 using H&E (FIG. 3A), p53 (FIG. 3B), Ki-67 (FIG. 3C), cytokeratin 7 (FIG. 3D), and uroplakin III (FIG. 3E) are all consistent with high-grade urothelial carcinoma. FIGS. 3F-J: Identical staining performed on fixed adherent cells grown on slides show similar staining pattern as parental tissue. FIGS. 3K-O: Identical staining on cultured human prostate cancer cells shows similar p53 and Ki-67 staining but no cytokeratin 7 or uroplakin III staining.

FIG. 4 shows the drug sensitivity profile for line #7. Drug sensitivity was performed after 24-hour drug exposure followed by MTT proliferation assay. Optical density from MTT assay is proportional to viable cells present. Mean optical densities with 95% confidence intervals for six technical replicates of each drug dilution are shown. Statistical comparisons were made between DMSO only (pink bar) and each drug dilution.

FIG. 5 shows tissue culture images of the bladder tumor organoid line MaB22 (passage 2) generated with the organoid culturing methodology described herein. Images are shown at 10× magnification.

FIG. 6 shows hematoxylin and eosin (H&E) staining of bladder tumor organoid line MaB22 (passage 2).

FIG. 7 shows immunofluorescent staining of bladder tumor organoid line MaB22 (passage 2) for CK7 and Ki67 as indicated. Images are shown at 40× magnification.

FIG. 8 shows immunofluorescent staining of bladder tumor organoid line MaB22 (passage 2) for CK8 and CK5 as indicated. Images are shown at 40× magnification.

FIG. 9 shows immunohistochemical staining of bladder tumor organoid line MaB22 (passage 2) for p53.

FIG. 10 shows histology of patient-derived bladder cancer organoids and corresponding parental tumors. Bright-field images of organoids in culture and hematoxylin-eosin (H&E) stained sections are shown for six independent patient-derived organoid lines, together with low and high-power images of H&E-stained sections from the corresponding parental tumors.

FIG. 11 shows marker expression in patient-derived organoids and parental tumors. (Top) Immunofluorescence staining for p53 (green), CK8 (red), CK5 (white), and DAPI (blue) in organoids from six independent patient-derived lines and in their corresponding parental tumors. All 6 lines display prevalent staining for the luminal marker CK8, and that MaB33 and JuB3 show strong nuclear p53 immunostaining. Notably, the MaB30 and SuB2 lines and their parental tumors show minor populations of CK5-positive cells (arrows), indicating phenotypic heterogeneity. (Bottom) Immunofluorescence staining for CK7 (green), Ki67 (red), and DAPI (blue) in organoid lines and parental tumors. All 6 lines display strong expression of CK7, consistent with their urothelial origin.

FIG. 12 shows a summary of targeted exome sequencing of patient-derived organoids. Sequencing analyses of seven patient-derived organoid lines together with their corresponding parental tumors and normal patient blood were performed using the MSK-IMPACT platform, and analyzed using a custom bioinformatic pipeline. Mutations (top) and copy number alterations (bottom) identified in the organoid lines are summarized using the indicated colors and symbols.

FIGS. 13-14 shows Tumor heterogeneity and evidence for clonal evolution in organoid culture. (FIG. 13) Partial output from cBioPortal, showing mutations identified in the JuB3 organoid line at passages 2, 6, and 10, as well as in the parental tumor. Multiple mutations are only found in the parental tumor (such as NF1 and PAK7), while several mutations are found in all four samples. Mutations in NTRK3 and SMARCA4 are only detected at passage 2 and in the parental tumor (arrows), and are subclonal (see allelic frequencies column). (FIG. 14) Marker expression in JuB3 organoids at passage 6. Note heterogeneity of the organoid population with respect to expression of CK14 and P-cadherin (arrows).

FIG. 15 shows xenografts derived from patient-derived organoids by orthotopic implantation. (Left) Ultrasound imaging of orthotopic implants of organoids into the bladder wall. (Right) Histopathological analysis of xenograft and corresponding organoid and parental tumor tissue. Note that a CK5-positive subpopulation of tumor cells is present in all three samples (arrows), consistent with persistence of tumor heterogeneity.

FIG. 16 shows organoids established from patient-derived xenografts. The similarity of marker expression in xenograft tissue and in organoids derived from the xenograft is shown by immunofluorescence (left) for p53 (green), CK8 (red), CK5 (white), and DAPI (blue) or (right) for CK7 (green), Ki67 (red), and DAPI (blue).

FIG. 17 shows drug response assays using patient-derived organoids. Dose response curves are shown for three independent patient-derived organoid lines treated with the indicated compounds. Calculated values for IC₅₀ and area under the curve (AUC) are shown for each combination of organoid line and treatment. Organoids were plated at a concentration of 2,000 cells per well on 96-well plates, and treated for 5 days with the indicated drug concentration, followed by CellTiterGlo assays (Promega) to measure cell viability. Each data point corresponds to three biological replicates; error bars correspond to one standard deviation.

FIGS. 18-19 shows response of organoid lines to drugs that target epigenetic regulators.

FIG. 18 shows a graph of Log concentration of drug vs. percentage viability of the organoid lines indicated.

FIG. 19 shows the calculated values for IC₅₀ and area under the curve (AUC) for each organoid line.

FIG. 20 shows clinical challenges associated with bladder cancer. (Top) Proposed progression pathway for bladder cancer. Possible relationships between low-grade and high-grade disease are indicated. (Bottom) Schematic of clinical stages and standard treatments for bladder cancer. TUR, transurethral resection; CIS, carcinoma in situ. Adapted from [6]

FIGS. 21A-21D shows ultrasound-guided intramural engraftment into bladder for propagation of tumors. (A) Experimental design. UMUC3 human bladder cancer cells are implanted orthotopically into the bladder of host mice and tumor growth was monitored using ultrasound imaging. Cisplatin treatment was initiated when tumors reached 5 mm, and mice were treated (8 mg/kg) for 2 weeks. (B) Phenotypic analyses of UMUC3 human bladder tumors. Shown are representative images of whole mount tumors, ultrasound images, H&E staining, or immunostaining with the indicated markers. The numbers on the ultrasound images show tumor volume; scale bars represent 50 microns. (C) Summary of tumor weights for the indicated groups. n=9-14/group; p-values were calculated using a Mann Whitney U test. (D) Quantification of cellular proliferation as assessed by the Ki67 staining of tumor cells. n=3/group; p-values were calculated using a Mann Whitney U test.

FIG. 22 shows drug response assays using patient-derived organoids. Dose response curves are shown for six independent patient-derived organoid lines treated with the indicated compounds. Calculated values for IC50 and area under the curve (AUC) are shown for each combination of organoid line and treatment. Organoids were plated at a concentration of 2,000 cells per well on 96-well plates, and treated for 5 days with the indicated drug concentration, followed by CellTiterGlo assays (Promega) to measure cell viability. Each data point corresponds to three biological replicates; error bars correspond to one standard deviation.

FIG. 23 shows histology of patient-derived bladder cancer organoids and corresponding parental tumors. Bright-field images of organoids in culture and hematoxylin-eosin (H&E) stained sections are shown for patient-derived organoid lines as indicated, together with low and high-power images of H&E-stained sections from the corresponding parental tumors.

FIG. 24 shows marker expression in JuB3 patient-derived organoids and parental tumors.

FIG. 25 shows marker expression in MaB28 patient-derived organoids and parental tumors.

FIG. 26 shows marker expression in MaB30 patient-derived organoids and parental tumors.

FIG. 27 shows marker expression in MaB30-2 patient-derived organoids and parental tumors.

FIG. 28 shows marker expression in SuB2 patient-derived organoids and parental tumors.

FIGS. 29-41 show the response of organoid lines to drugs as indicated.

### DETAILED DESCRIPTION

### Definitions and Abbreviations

The term “FBS” designates fetal bovine serum.

The term “EGF” designates epidermal growth factor.

The term “DMEM” designates Dulbecco's Modified Eagle Medium.

The term “F-12” designates Nutrient Mixture F-12.

The term “HBSS” designates Hanks” Balanced Salt Solution.

The term “CK7” designates cytokeratin 7.

The term “UP3” designates uroplakin III.

The term “ROCK” designates Rho-Associated Coil Kinase.

The term “EpCAM” designates Epithelial Cell Adhesion Molecule.

The term “DMSO” designates dimethyl sulfoxide.

The term “TURBT” designates transurethral resection of bladder tumor.

The term “CK5” designates cytokeratin 5.

The term “CK8” designates cytokeratin 8.

The term “PBS” designates Phosphate Buffered Saline.

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

### DETAILED DESCRIPTION

The present invention relates to a methodology for the culture of bladder cell lines and organoids from human bladder, both non-cancerous as well as cancer tissue. For example, the present invention also relates to a new protocol to rapidly and efficiently establish patient-derived organoids and cell lines from bladder tumor biopsy specimens. These organoids and cell lines can be used to predict individual response to chemotherapeutic agents, as well as test new agents in a preclinical setting. Previous work in the field has not been successful in culturing patient specific bladder tissue for determining the response that an individual's own tumor cells will have in a clinical setting. Human bladder tumors have been used to establish cell cultures. Without being bound by theory, the published efficiency rates with which cultures can be successfully established are widely variable (31-78%) and generally far from optimal. Most of these studies only culture the cells for a short period of time, limiting the long-term utility. Another limitation is that many studies use tissue from cystectomy, which requires removal of the entire bladder. Removal of the entire bladder is not useful for testing of intravesical agents which involves administration of treatment directly into the bladder.

In one embodiment, the methodology described herein allows a small sample (as small as 20 milligrams) to be taken from an endoscopic bladder biopsy or transurethral resection of bladder tumor (TURBT) and grow it in culture. In one embodiment, the methodology described herein has a very high efficiency rate (currently 89%) and causes the cells to grow very rapidly, providing enough cells to perform sensitivity testing in as little as two weeks. Since intravesical therapy is typically started 2-6 weeks after TURBT, this allows analysis of the use of intravesical agents within a useful timeframe. In another embodiment, the bladder cell lines can remain in culture for an extended period of time, and they can also be frozen for long-term storage and thawed at a later date with immediate resumption of normal growth.

In some embodiments, the present invention relates to culture conditions that can support the growth of dissociated bladder epithelial cells to form large tissue masses (organoids) in culture. This can be achieved using cells from human patient specimens (using fresh bladder tissue).

In some embodiments, the present invention relates to the growth of cell lines and organoids from normal human bladder tissue from endoscopic bladder biopsy, TURBT, or cystectomy, as well as any human bladder cancer tissue from these procedures.

In some embodiments, the cell lines and organoids of the present invention maintain the transformed phenotype of the bladder tumor tissue.

In one aspect, the invention provides a method for culturing a bladder cell line, a bladder tumor cell line, a bladder organoid, or a bladder tumor organoid, wherein the cell line or organoid maintains or displays the phenotype of the sample of bladder tissue from which the cell line or organoid is derived. The phenotype of the cell line or organoid can be determined by evaluating markers. Expression of markers can be evaluated by a variety of methods known in the art. The presence of markers can be determined at the DNA, RNA or polypeptide level. In one embodiment, the method can comprise detecting the presence of a marker gene polypeptide expression. Polypeptide expression includes the presence or absence of a marker gene polypeptide sequence. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (such as, p53, Ki-67, CK7, UP3, CK5, CK8, or a combination thereof) RNA expression, for example in bladder cell lines or organoids. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

In one embodiment, organoids can display characteristic tissue architecture. The method can comprise detecting other characteristic tissue architecture in organoids using various techniques known in the art, including staining of tissue with various stains including, but not limited to, Gomori's trichrome, haematoxylin and eosin, periodic acid-Schiff, Masson's trichrome, Silver staining, or Sudan staining.

In some embodiments, the present invention relates to screening methods for the identification of new candidate therapeutics for bladder cancer. This screening can be performed on a patient-specific basis using cell lines or organoids grown from surgically-isolated tumor tissue.

In some embodiments, the present invention relates to small molecule screens for the identification of candidate therapeutics.

In some embodiments, the present invention relates to tumor tissue banks in which patient-specific cell lines or organoids can be stored and used for the large-scale screening of candidate therapeutic compounds. Such cell line or organoid banks can also be useful for patient-specific diagnostics, assays for the efficacy of potential treatments, and identification of the appropriate targeted tumor population, as well as other applications in personalized medicine.

The culture conditions of the instant invention can include EGF, 5% fetal bovine serum, and 5% Matrigel.

Matrigel™ is the trade name for a reconstituted basement membrane preparation that is extracted from the Engelbreth-Holm-Swarm (EHS) mouse sarcoma, a tumor rich in extracellular matrix proteins. This material, once isolated, is approximately 60% laminin, 30% collagen IV, and 8% entactin. Entactin is a bridging molecule that interacts with laminin and collagen IV, and contributes to the structural organization of these extracellular matrix molecules. Matrigel also containsheparan sulfate proteoglycan (perlecan), TGF-β, epidermal growth factor, insulinlike growth factor, fibroblast growth factor, tissue plasminogen activator, and other growth factors which occur naturally in the EHS tumor. There is also residual matrix metalloproteinases derived from the tumor cells. Matrigel is produced and sold by Corning Life Sciences. Trevigen, Inc. markets their own version under the trade name Cultrex BME.

In some embodiments, organoids of the invention can be cultured in a Matrigel™ gel or matrix. In another embodiment, the organoids of the invention can be cultures in a collagen matrix.

In some embodiments, the cell lines and organoids provide a methodology for the culture and long-term maintenance of viable human bladder cancer tissue. The availability of this methodology allows many applications for tumor screening and experimental therapeutics in an ex vivo culture-based setting, providing patient-specific reagents to investigate tumor response without the use of elaborate mouse models or extensive clinical trials.

The present invention provides methods for culturing bladder tissue. In one aspect the present invention provides methods for culturing bladder tissue that maintains the differentiated state of bladder, or recapitulates the phenotype of bladder tumors.

In one embodiment, the bladder cancer is a transitional cell carcinoma or a urothelial cell carcinoma. In another embodiment, the bladder cancer is a squamous cell carcinoma. In another embodiment, the bladder cancer is adenocarcinoma. In one embodiment, the epithelium of the bladder is a transitional epithelium or urothelium.

### Methods of Culturing Bladder Cell Lines

In one aspect, the invention provides a method for culturing a bladder cell line, the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture.

Cells can be grown in suspension or adherent cultures. Some cells can be cultured without being attaching to a surface (suspension cultures), while other cells require a surface (adherent cells). Cells can also grow in a three-dimensional environment such as a matrix or a scaffold.

In one embodiment, the bladder cell lines grow as attached cells in two-dimensional culture. In one embodiment, the bladder cell lines grow as adherent cells. In one embodiment, the adherent cell culture support is a tissue culture plate. Tissue culture plates and supports can be used in a variety of shapes, sizes and materials, including, but not limited to, plates, flasks, wells, and bags. Tissue culture supports can be coated with various substances, including, but not limited to, extracellular matrix components to increase adhesion properties for example. In one embodiment, the adherent cell culture support is a tissue culture plate that enhances or maximizes attachment of the cells to the surface of the support. In one embodiment, the adherent cell culture support is a Primaria™ surface modified cell culture plate. In one embodiment, the adherent cell culture support is a Primaria™ 24 well flat bottom surface modified multiwell cell culture plate. The Primaria™ surface modified cell culture plate is an example of a type of tissue culture support that enhances or maximizes attachment of the cells to the surface of the support. A variety of alternative cell culture supports that enhance or maximize attachment of cells to the surface of the support are known in the art and can be found, for example, in Corning Cell Culture Selection Guide, the contents of which is hereby incorporated by reference in its entirety. In another embodiment, the adherent cell culture support is a polystyrene plate. In a further embodiment, the adherent cell culture support is a surface modified polystyrene plate. For example, the surface of the plate can be modified to incorporate anionic and cationic functional groups to enhance the attachment of the cells to the surface of the support. In one embodiment, the adherent cell culture support is a 6 well plate, a 12 well plate, a 24 well plate, a 48 well plate, or a 96 well plate.

In one embodiment, the bladder tissue is non-cancerous. In another embodiment, the bladder tissue is cancerous. In another embodiment, the bladder tissue is obtained from a bladder tumor. In a further embodiment, the subject is a human. In another embodiment, the bladder tissue is obtained from an endoscopic biopsy, an endoscopic resection, or a cystectomy sample. In a further embodiment, the bladder cell line displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% Matrigel. In another embodiment, the culture medium comprises 5% heat-inactivated charcoal stripped FBS. In another embodiment, the ROCK inhibitor is Y-27632. In another embodiment, the culture medium comprises 10 μM of Y-27632. In one embodiment, the cells in the bladder cell line grow as attached cells in two-dimensional culture. In another embodiment, a single cell suspension is obtained by the dissociating of (b). In a further embodiment, the single cell suspension contains epithelial and stromal cells. In another embodiment, (b) comprises dissociating the sample of bladder tissue with collagenase, hyaluronidase, dispase, or a combination thereof. In another embodiment, the isolating of (c) is by immunomagnetic cell separation. In a further embodiment, the immunomagnetic cell separation uses an antibody against Epithelial Cell Adhesion Molecule (EpCAM). In one embodiment, the method further comprises: (e) serially passaging the bladder cell line colonies.

The present invention provides methods for dissociating cells from a tissue or mixed population of cells. In one embodiment, cells are dissociated from bladder tissue.

In one embodiment, cells are dissociated from normal tissue. In one embodiment, cells are dissociated from non-cancerous tissue. In another embodiment, cells are dissociated from cancerous tissue. In another embodiment, cells are dissociated from human tissue. In one embodiment, cells are dissociated from localized tumors. In another embodiment, cells are dissociated from malignant tumors. In another embodiment, cells are dissociated from metastasized tumors.

In a further embodiment, the bladder cell lines are cultured from one or more localized tumors. In one embodiment, the bladder cell lines are cultured from malignant tumors. In another embodiment, the bladder cell lines are cultured from metastasized tumors. In one embodiment, the tumor is a bladder tumor.

In one embodiment, a sample of tissue can be obtained by biopsy. Methods of obtaining tissue samples are known to one of skill in the art. In one embodiment, the sample of tissue is obtained from a bladder biopsy or endoscopic resection. In another embodiment, the sample of tissue is obtained from a cystectomy.

In one embodiment, the subject is an animal. In other embodiments, the subject is a human. In other embodiments, the subject is a mammal. In some embodiments, the subject is a rodent, such as a mouse or a rat. In some embodiments, the subject is a cow, pig, sheep, goat, cat, horse, dog, and/or any other species of animal used as livestock or kept as pets.

In one aspect, the invention provides a method for culturing a bladder cell line or a bladder tumor cell line, wherein the cell line maintains or displays the phenotype of the sample of bladder tissue from which the cell line is derived. The phenotype of the cell line can be determined by evaluating markers. Expression of markers can be evaluated by a variety of methods known in the art. In one embodiment, the bladder cell lines display the differentiation of the non-cancerous bladder tissue. In one embodiment, the bladder cell lines display the transformed phenotype of the cancerous bladder tissue.

In one embodiment, the culture medium comprises EGF. In another embodiment, the culture medium does not comprise EGF. In one embodiment, the culture medium comprises Glutamax. In another embodiment, the culture medium does not comprise Glutamax. In one embodiment, the culture medium comprises antibiotic-antimycotic. In another embodiment, the culture medium does not comprise antibiotic-antimycotic.

In one embodiment, the culture medium comprises serum, including, but not limited to, FBS. In another embodiment, the culture medium does not comprise serum, including, but not limited to, FBS. In one embodiment, the culture medium comprises a ROCK inhibitor. In another embodiment, the culture medium does not comprise a ROCK inhibitor. In one embodiment, the culture medium comprises Matrigel. In another embodiment, the culture medium does not comprise Matrigel.

In one embodiment, the bladder cell lines grow as attached cells in two-dimensional culture. In one embodiment, the cells are cancerous. In another embodiment, the cells are tumor cells. In another embodiment, the cells are normal. In yet another embodiment, the cells are non-cancerous.

In another embodiment, the cell cultures are used as cell lines. In one embodiment, the cell cultures are used as bladder cell lines. In one embodiment, the cell cultures are used as cancer cell lines. In another embodiment, the cell cultures are used as bladder cancer cell lines.

In one embodiment, the cells of the bladder cell lines express p53, Ki-67, CK7, UP3, CK5, CK8, or a combination thereof. In one embodiment, the cells of the bladder cell lines express p53. In another embodiment, the cells of the bladder cell lines express Ki-67. In another embodiment, the cells of the bladder cell lines express CK7. In another embodiment, the cells of the bladder cell lines express UP3. In another embodiment, the cells of the bladder cell lines express CK5. In another embodiment, the cells of the bladder cell lines express CK8.

In one aspect, the invention provides a method for culturing a bladder cell line or a bladder tumor cell line, wherein the method has a high efficiency rate. In one aspect, the invention provides a high efficiency method for culturing a bladder cell line or a bladder tumor cell line. In one embodiment, the efficiency rate is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%.

In one embodiment, the invention provides a method for culturing a bladder cell line or a bladder tumor cell line, wherein the method has at least 80% efficiency. In one embodiment, the invention provides a method for culturing a bladder cell line or a bladder tumor cell line, wherein the method has at least 85% efficiency. In one embodiment, the invention provides a method for culturing a bladder cell line or a bladder tumor cell line, wherein the method has at least 89% efficiency. In one embodiment, the invention provides a method for culturing a bladder cell line or a bladder tumor cell line, wherein the method has at least 90% efficiency.

### Methods of Culturing Bladder Organoids by Matrigel Floating Method

In one aspect, the invention provides a method for culturing a bladder organoid, the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture.

In one embodiment, the bladder cell lines grow as organoids in Matrigel floating culture. In one embodiment, the low attachment cell culture support is a tissue culture plate. Tissue culture plates and supports can be used in a variety of shapes, sizes and materials, including, but not limited to, plates, flasks, wells, and bags. Tissue culture supports can be coated with various substances, to decrease adhesion properties. In another embodiment, the low attachment cell culture support is a tissue culture plate that minimizes or prevents attachment of the cells to the surface of the support. In one embodiment, the low attachment cell culture support is a Corning Ultra-Low Attachment cell culture plate. In one embodiment, the low attachment cell culture support is a Corning Ultra-Low Attachment 96 well plate. The Corning Ultra-Low Attachment cell culture plate is an example of a type of tissue culture support that minimizes or prevents attachment of the cells to the surface of the support. A variety of alternative cell culture supports that minimize or prevent attachment of cells to the surface of the support are known in the art and can be found, for example, in Corning Cell Culture Selection Guide, the contents of which is hereby incorporated by reference in its entirety. In another embodiment, the low attachment cell culture support is a polystyrene plate. In a further embodiment, the low attachment cell culture support is a surface modified polystyrene plate. For example, the surface of the support can be modified to be hydrophilic and/or neutrally charged to minimize or prevent the attachment of the cells to the surface of the support. In another embodiment, the surface of the support can be modified so the plate has a covalently bonded hydrogel surface to minimize or prevent the attachment of the cells to the surface if the plate. In one embodiment, the low attachment cell culture support is a 6 well plate, a 12 well plate, a 24 well plate, a 48 well plate, or a 96 well plate.

In one embodiment, the bladder tissue is non-cancerous. In another embodiment, the bladder tissue is cancerous. In another embodiment, the bladder tissue is obtained from a bladder tumor. In a further embodiment, the subject is a human. In another embodiment, the bladder tissue is obtained from an endoscopic biopsy, an endoscopic resection, or a cystectomy sample. In a further embodiment, the bladder organoid displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% Matrigel. In another embodiment, the culture medium comprises 5% heat-inactivated charcoal stripped FBS. In another embodiment, the ROCK inhibitor is Y-27632. In another embodiment, the culture medium comprises 10 μM of Y-27632. In one embodiment, a bladder cell line is obtained from the organoids. In one embodiment, the cells in the bladder cell line grow as attached cells in two-dimensional culture. In another embodiment, a single cell suspension is obtained by the dissociating of (b). In a further embodiment, the single cell suspension contains epithelial and stromal cells. In another embodiment, (b) comprises dissociating the sample of bladder tissue with collagenase, hyaluronidase, dispase, or a combination thereof. In another embodiment, the isolating of (c) is by immunomagnetic cell separation. In a further embodiment, the immunomagnetic cell separation uses an antibody against Epithelial Cell Adhesion Molecule (EpCAM). In one embodiment, the method further comprises: (e) serially passaging the bladder cell line colonies.

The present invention provides methods for dissociating cells from a tissue or mixed population of cells. In one embodiment, cells are dissociated from bladder tissue.

In one embodiment, cells are dissociated from normal tissue. In one embodiment, cells are dissociated from non-cancerous tissue. In another embodiment, cells are dissociated from cancerous tissue. In another embodiment, cells are dissociated from human tissue. In one embodiment, cells are dissociated from localized tumors. In another embodiment, cells are dissociated from malignant tumors. In another embodiment, cells are dissociated from metastasized tumors.

In a further embodiment, the organoids are cultured from one or more localized tumors. In one embodiment, the organoids are cultured from malignant tumors. In another embodiment, the organoids are cultured from metastasized tumors. In one embodiment, the tumor is a bladder tumor.

In one embodiment, a sample of tissue can be obtained by biopsy. Methods of obtaining tissue samples are known to one of skill in the art. In one embodiment, the sample of tissue is obtained from a bladder biopsy or endoscopic resection. In another embodiment, the sample of tissue is obtained from a cystectomy.

In one embodiment, the subject is an animal. In other embodiments, the subject is a human. In other embodiments, the subject is a mammal. In some embodiments, the subject is a rodent, such as a mouse or a rat. In some embodiments, the subject is a cow, pig, sheep, goat, cat, horse, dog, and/or any other species of animal used as livestock or kept as pets.

In one aspect, the invention provides a method for culturing a bladder organoid or a bladder organoid, wherein the organoid maintains or displays the phenotype of the sample of bladder tissue from which the organoid is derived. The phenotype of the organoid can be determined by evaluating markers. Expression of markers can be evaluated by a variety of methods known in the art. In one embodiment, the organoids display the differentiation of the non-cancerous bladder tissue. In one embodiment, the organoids display the transformed phenotype of the cancerous bladder tissue.

In one embodiment, the culture medium comprises EGF. In another embodiment, the culture medium does not comprise EGF. In one embodiment, the culture medium comprises Glutamax. In another embodiment, the culture medium does not comprise Glutamax. In one embodiment, the culture medium comprises antibiotic-antimycotic. In another embodiment, the culture medium does not comprise antibiotic-antimycotic.

In one embodiment, the culture medium comprises serum, including, but not limited to, FBS. In another embodiment, the culture medium does not comprise serum, including, but not limited to, FBS. In one embodiment, the culture medium comprises a ROCK inhibitor. In another embodiment, the culture medium does not comprise a ROCK inhibitor. In one embodiment, the culture medium comprises Matrigel. In another embodiment, the culture medium does not comprise Matrigel.

In one embodiment, bladder cell lines that grow as attached cells in two-dimensional culture are derived from the organoids. In one embodiment, the cells are cancerous. In another embodiment, the cells are tumor cells. In another embodiment, the cells are normal. In yet another embodiment, the cells are non-cancerous.

In one embodiment, organoids can be converted to two-dimensional adherent culture by passaging the organoid culture and plating the dissociated bladder organoid cells on an adherent cell culture support. In one embodiment, the adherent cell culture support is a tissue culture plate. Tissue culture plates and supports can be used in a variety of shapes, sizes and materials. Tissue culture plates can be coated with various substances, including, but not limited to, extracellular matrix components to increase adhesion properties for example. In another embodiment, the adherent cell culture support is a tissue culture plate that enhances or maximizes attachment of the cells to the surface of the support. In one embodiment, the adherent cell culture support is a Primaria™ 24 well flat bottom surface modified multiwell cell culture plate. The Primaria™ 24 well flat bottom surface modified multiwell cell culture plate is an example of a type of tissue culture plate that enhances or maximizes attachment of the cells to the surface of the support. A variety of alternative cell culture plates that enhance or maximize attachment of cells to the surface of the support are known in the art and can be found, for example, in Corning Cell Culture Selection Guide, the contents of which is hereby incorporated by reference in its entirety. In another embodiment, the adherent cell culture support is a polystyrene plate. In a further embodiment, the adherent cell culture support is a surface modified polystyrene plate. For example, the surface of the plate can be modified to incorporate anionic and cationic functional groups to enhance the attachment of the cells to the surface of the support. In one embodiment, the cell culture support is a 6 well plate, a 12 well plate, a 24 well plate, a 48 well plate, or a 96 well plate. In another embodiment, the cell cultures are used as cell lines. In one embodiment, the cell cultures are used as bladder cell lines. In one embodiment, the cell cultures are used as cancer cell lines. In another embodiment, the cell cultures are used as bladder cancer cell lines.

In one embodiment, cell cultures are obtained from the organoids. In another embodiment, cells in the cell cultures grow as attached cells in two-dimensional culture. In yet another embodiment, the cell cultures comprise cell lines. In one embodiment, the cells are cancerous. In another embodiment, the cells are tumor cells. In another embodiment, the cells are normal. In yet another embodiment, the cells are non-cancerous.

In one embodiment, the cell cultures comprise bladder cell lines. In one embodiment, the cell cultures comprise cancer cell lines. In another embodiment, the cell cultures comprise bladder cancer cell lines.

In one embodiment, the cells of the organoids express p53, Ki-67, CK7, UP3, CK5, CK8, or a combination thereof. In one embodiment, the cells of the organoids express p53. In another embodiment, the cells of the organoids express Ki-67. In another embodiment, the cells of the organoids express CK7. In another embodiment, the cells of the organoids express UP3. In another embodiment, the cells of the bladder cell lines express CK5. In another embodiment, the cells of the bladder cell lines express CK8.

In one embodiment, the cells of the bladder cell lines express p53, Ki-67, CK7, UP3, CK5, CK8 or a combination thereof. In one embodiment, the cells of the bladder cell lines express p53. In another embodiment, the cells of the bladder cell lines express Ki-67. In another embodiment, the cells of the bladder cell lines express CK7. In another embodiment, the cells of the bladder cell lines express UP3. In another embodiment, the cells of the bladder cell lines express CK5. In another embodiment, the cells of the bladder cell lines express CK8.

In one aspect, the invention provides a method for culturing a bladder organoid or a bladder tumor organoid, wherein the method has a high efficiency rate. In one aspect, the invention provides a high efficiency method for culturing a bladder organoid or a bladder tumor organoid. In one embodiment, the efficiency rate is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%.

In one embodiment, the invention provides a method for culturing a bladder organoid or a bladder tumor organoid, wherein the method has at least 80% efficiency. In one embodiment, the invention provides a method for culturing a bladder organoid or a bladder tumor organoid, wherein the method has at least 85% efficiency. In one embodiment, the invention provides a method for culturing a bladder organoid or a bladder tumor organoid, wherein the method has at least 89% efficiency. In one embodiment, the invention provides a method for culturing a bladder organoid or a bladder tumor organoid, wherein the method has at least 90% efficiency.

### Methods of Culturing Bladder Organoids by Embedding Method

In one aspect, the invention provides a method for culturing a bladder organoid, the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids.

In one embodiment, the bladder cell lines grow as organoids in Matrigel embedding culture. In one embodiment, the cell culture support is a tissue culture plate. In one embodiment, the cell culture support is a 6-well tissue culture plate. Tissue culture plates and supports can be used in a variety of shapes, sizes and materials, including, but not limited to, plates, flasks, wells, and bags. A variety of cell culture supports are known in the art and can be found, for example, in Corning Cell Culture Selection Guide, the contents of which is hereby incorporated by reference in its entirety. In another embodiment, the cell culture support is a polystyrene plate. In a further embodiment, the cell culture support is a surface modified polystyrene plate. In one embodiment, the cell culture support is a 6 well plate, a 12 well plate, a 24 well plate, a 48 well plate, or a 96 well plate.

In one embodiment, the bladder tissue is non-cancerous. In another embodiment, the bladder tissue is cancerous. In another embodiment, the bladder tissue is obtained from a bladder tumor. In a further embodiment, the subject is a human. In another embodiment, the bladder tissue is obtained from an endoscopic biopsy, an endoscopic resection, or a cystectomy. In a further embodiment, the bladder organoid displays the transformed phenotype of the cancerous bladder tissue. In one embodiment, the culture medium further comprises Glutamax. In another embodiment, the culture medium further comprises EGF. In a further embodiment, the culture medium further comprises antibiotic-antimycotic. In another embodiment, the culture medium comprises 10 ng/ml of EGF. In another embodiment, the culture medium comprises 5% heat-inactivated charcoal stripped FBS. In another embodiment, the culture medium contains a ROCK inhibitor. In another embodiment, the ROCK inhibitor is Y-27632. In another embodiment, the culture medium comprises 10 μM of Y-27632. In one embodiment, a bladder cell line is obtained from the organoids. In one embodiment, the cells in the bladder cell line grow as attached cells in two-dimensional culture. In another embodiment, cell clusters are obtained by the dissociating of (b). In another embodiment, a single cell suspension is obtained by the dissociating of (b). In a further embodiment, the single cell suspension contains epithelial and stromal cells. In another embodiment, (b) comprises dissociating the sample of bladder tissue with collagenase, hyaluronidase, dispase, or a combination thereof. In another embodiment, (b) comprises dissociating the sample of bladder tissue with collagenase and hyaluronidase. In another embodiment, (b) comprises dissociating the sample of bladder tissue with trypsin. In another embodiment, (b) comprises dissociating the sample of bladder tissue with TrypLE™. In another embodiment, (b) comprises dissociating the sample of bladder tissue with collagenase and hyaluronidase followed by trypsin. In another embodiment, (b) comprises dissociating the sample of bladder tissue with collagenase and hyaluronidase followed by TrypLE™. In one embodiment, the method further comprises: (f) serially passaging the bladder organoids. In one embodiment, the bladder organoids are passaged using dispase.

In another embodiment, the dissociating of (b) is followed by an isolation step, wherein dissociated bladder epithelial cells are isolated from the dissociated bladder tissue of (b). In one embodiment the isolating of bladder epithelial cells is by immunomagnetic cell separation. In a further embodiment, the immunomagnetic cell separation uses an antibody against Epithelial Cell Adhesion Molecule (EpCAM).

In one embodiment, the contacting of (c) is performed below about 10° C. in order to maintain the Matrigel solution in liquid form. After plating in the cell culture support the temperature can be raised above about 10° C. and the Matrigel solution can form a matrix or gel. In one embodiment, the Matrigel solution solidifies or forms a gel by incubation at 37° C. for 30 minutes. In one embodiment, the Matrigel solution solidifies or forms a gel at about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In another embodiment, before plating the dissociated bladder tissue and Matrigel solution in the cell culture support, the cell culture support is surface modified. In one embodiment, the support surface is pre-coated by rinsing Matrigel solution over the support surface and incubating the cell culture support at 37° C. for at least 30 minutes. In one embodiment, the Matrigel solution comprises hepatocyte medium and Matrigel. In one embodiment, the Matrigel solution comprises serum, including, but not limited to, FBS. In another embodiment, the Matrigel solution does not comprise serum, including, but not limited to, FBS. In one embodiment, the Matrigel solution comprises 3 parts Matrigel to 2 parts hepatocyte medium. In one embodiment, the Matrigel solution comprises 60% Matrigel and 40% hepatocyte medium.

In one aspect, the invention provides a method for culturing a bladder organoid, the method comprising: (a) obtaining a sample of bladder tissue from a subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; and (e) incubating the culture of (d) wherein the dissociated bladder tissue forms organoids.

In one embodiment, the bladder cell lines grow as organoids in a collagen matrix. In one embodiment, the bladder cell lines grow as organoids in an extracellular matrix or scaffold, including, but not limited to collagen, laminin, fibronectin, gelatin, or Geltrex®. In one embodiment, the collagen matrix comprises collagen I. In one embodiment, the collagen matrix comprises rat tail collagen I.

In one embodiment, after plating in the cell culture support the temperature can be raised above about 10° C. and the collagen solution can form a matrix or gel. In one embodiment, the collagen solution solidifies or forms a gel by incubation at 37° C. for 30 minutes. In one embodiment, the Matrigel solution solidifies or forms a gel at about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

In another embodiment, before plating the dissociated bladder tissue and collagen solution in the cell culture support, the cell culture support is surface modified. In one embodiment, the support surface is pre-coated by rinsing collagen solution over the support surface and incubating the cell culture support at 37° C. for at least 30 minutes. In one embodiment, the collagen solution comprises setting solution and collagen. In one embodiment, the collagen solution comprises 9 parts collagen to 1 parts setting solution. In one embodiment, setting solution comprises EBSS, sodium bicarbonate and sodium hydroxide.

The present invention provides methods for dissociating cells from a tissue or mixed population of cells. In one embodiment, cells are dissociated from bladder tissue.

In one embodiment, cells are dissociated from normal tissue. In one embodiment, cells are dissociated from non-cancerous tissue. In another embodiment, cells are dissociated from cancerous tissue. In another embodiment, cells are dissociated from human tissue. In one embodiment, cells are dissociated from localized tumors. In another embodiment, cells are dissociated from malignant tumors. In another embodiment, cells are dissociated from metastasized tumors.

In a further embodiment, the organoids are cultured from one or more localized tumors. In one embodiment, the organoids are cultured from malignant tumors. In another embodiment, the organoids are cultured from metastasized tumors. In one embodiment, the tumor is a bladder tumor.

In one embodiment, a sample of tissue can be obtained by biopsy. Methods of obtaining tissue samples are known to one of skill in the art. In one embodiment, the sample of tissue is obtained from a bladder biopsy or endoscopic resection. In another embodiment, the sample of tissue is obtained from a cystectomy.

In one embodiment, the subject is an animal. In other embodiments, the subject is a human. In other embodiments, the subject is a mammal. In some embodiments, the subject is a rodent, such as a mouse or a rat. In some embodiments, the subject is a cow, pig, sheep, goat, cat, horse, dog, and/or any other species of animal used as livestock or kept as pets.

In one aspect, the invention provides a method for culturing a bladder organoid or a bladder organoid, wherein the organoid maintains or displays the phenotype of the sample of bladder tissue from which the organoid is derived. The phenotype of the organoid can be determined by evaluating markers. Expression of markers can be evaluated by a variety of methods known in the art. In one embodiment, the organoids display the differentiation of the non-cancerous bladder tissue. In one embodiment, the organoids display the transformed phenotype of the cancerous bladder tissue.

In one embodiment, the liquid culture medium comprises EGF. In another embodiment, the liquid culture medium does not comprise EGF. In one embodiment, the liquid culture medium comprises Glutamax. In another embodiment, the liquid culture medium does not comprise Glutamax. In one embodiment, the liquid culture medium comprises antibiotic-antimycotic. In another embodiment, the liquid culture medium does not comprise antibiotic-antimycotic.

In one embodiment, the liquid culture medium comprises serum, including, but not limited to, FBS. In another embodiment, the liquid culture medium does not comprise serum, including, but not limited to, FBS. In one embodiment, the liquid culture medium comprises a ROCK inhibitor. In another embodiment, the liquid culture medium does not comprise a ROCK inhibitor.

In one embodiment, the Matrigel solution comprises hepatocyte medium and Matrigel. In one embodiment, the Matrigel solution comprises serum, including, but not limited to, FBS. In another embodiment, the Matrigel solution does not comprise serum, including, but not limited to, FBS. In one embodiment, the Matrigel solution comprises 3 parts Matrigel to 2 parts hepatocyte medium. In one embodiment, the Matrigel solution comprises 60% Matrigel and 40% hepatocyte medium.

In one embodiment, bladder cell lines that grow as attached cells in two-dimensional culture are derived from the organoids. In one embodiment, the cells are cancerous. In another embodiment, the cells are tumor cells. In another embodiment, the cells are normal. In yet another embodiment, the cells are non-cancerous.

In one embodiment, organoids can be converted to two-dimensional adherent culture by passaging the organoid culture and plating the dissociated bladder organoid cells on an adherent cell culture support. In one embodiment, the adherent cell culture support is a tissue culture plate. Tissue culture plates and supports can be used in a variety of shapes, sizes and materials. Tissue culture plates can be coated with various substances, including, but not limited to, extracellular matrix components to increase adhesion properties for example. In another embodiment, the adherent cell culture support is a tissue culture plate that enhances or maximizes attachment of the cells to the surface of the support. In one embodiment, the adherent cell culture support is a Primaria™ 24 well flat bottom surface modified multiwell cell culture plate. The Primaria™ 24 well flat bottom surface modified multiwell cell culture plate is an example of a type of tissue culture plate that enhances or maximizes attachment of the cells to the surface of the support. A variety of alternative cell culture plates that enhance or maximize attachment of cells to the surface of the support are known in the art and can be found, for example, in Corning Cell Culture Selection Guide, the contents of which is hereby incorporated by reference in its entirety. In another embodiment, the adherent cell culture support is a polystyrene plate. In a further embodiment, the adherent cell culture support is a surface modified polystyrene plate. For example, the surface of the plate can be modified to incorporate anionic and cationic functional groups to enhance the attachment of the cells to the surface of the support. In one embodiment, the adherent cell culture support is a 6 well plate, a 12 well plate, a 24 well plate, a 48 well plate, or a 96 well plate.

In another embodiment, the cell cultures are used as cell lines. In one embodiment, the cell cultures are used as bladder cell lines. In one embodiment, the cell cultures are used as cancer cell lines. In another embodiment, the cell cultures are used as bladder cancer cell lines.

In one embodiment, cell cultures are obtained from the organoids. In another embodiment, cells in the cell cultures grow as attached cells in two-dimensional culture. In yet another embodiment, the cell cultures comprise cell lines. In one embodiment, the cells are cancerous. In another embodiment, the cells are tumor cells. In another embodiment, the cells are normal. In yet another embodiment, the cells are non-cancerous.

In one embodiment, the cell cultures comprise bladder cell lines. In one embodiment, the cell cultures comprise cancer cell lines. In another embodiment, the cell cultures comprise bladder cancer cell lines.

In one embodiment, the cells of the organoids express p53, Ki-67, CK7, UP3, CK5, CK8, or a combination thereof. In one embodiment, the cells of the organoids express p53. In another embodiment, the cells of the organoids express Ki-67. In another embodiment, the cells of the organoids express CK7. In another embodiment, the cells of the organoids express UP3. In another embodiment, the cells of the bladder cell lines express CK5. In another embodiment, the cells of the bladder cell lines express CK8.

In one embodiment, the cells of the bladder cell lines express p53, Ki-67, CK7, UP3, CK5, CK8 or a combination thereof. In one embodiment, the cells of the bladder cell lines express p53. In another embodiment, the cells of the bladder cell lines express Ki-67. In another embodiment, the cells of the bladder cell lines express CK7. In another embodiment, the cells of the bladder cell lines express UP3. In another embodiment, the cells of the bladder cell lines express CK5. In another embodiment, the cells of the bladder cell lines express CK8.

In one aspect, the invention provides a method for culturing a bladder organoid or a bladder tumor organoid, wherein the method has a high efficiency rate. In one aspect, the invention provides a high efficiency method for culturing a bladder organoid or a bladder tumor organoid. In one embodiment, the efficiency rate is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100%.

In one embodiment, the invention provides a method for culturing a bladder organoid or a bladder tumor organoid, wherein the method has at least 80% efficiency. In one embodiment, the invention provides a method for culturing a bladder organoid or a bladder tumor organoid, wherein the method has at least 85% efficiency. In one embodiment, the invention provides a method for culturing a bladder organoid or a bladder tumor organoid, wherein the method has at least 89% efficiency. In one embodiment, the invention provides a method for culturing a bladder organoid or a bladder tumor organoid, wherein the method has at least 90% efficiency.

Isolation of Cells from Tissue

The present invention provides methods for separating, enriching, isolating or purifying cells from a tissue or mixed population of cells. In one embodiment, the isolated cells are epithelial cells. In another embodiment, the isolated cells are bladder epithelial cells. In one embodiment, cells are dissociated from normal bladder specimens. In one embodiment, cells are dissociated from non-cancerous bladder specimens. In another embodiment, cells are dissociated from cancerous bladder specimens. In another embodiment, the isolated cells are a mixed population. In a further embodiment, the isolated cells are not a mixed population.

In one embodiment, the cells are dissociated from normal organ specimens. In another embodiment, the cells are dissociated from non-cancerous organ specimens. In another embodiment, the cells are dissociated from cancerous organ specimens.

In one embodiment, bladder tissue is collected during surgery including, but not limited to, during cystectomies, endoscopic resection and bladder biopsies. In one embodiment, the bladder tissue is normal. In another embodiment, the bladder tissue is cancerous. In another embodiment, the bladder tissue is non-cancerous. In another embodiment, the bladder epithelial cells are cancerous. In another embodiment, the bladder epithelial cells is non-cancerous. In one embodiment, the bladder tissue is collected from a human subject.

In one embodiment the tissue sample is a bladder tissue sample. In another embodiment 1 gram of tissue is used. In one embodiment, at least 0.1 gram, at least 0.2 grams, at least 0.3 grams, at least 0.4 grams, at least 0.5 grams, at least 0.6 grams, at least 0.7 grams, at least 0.8 grams, at least 0.9 grams, at least 1.0 grams, at least 2.0 grams, at least 3.0 grams, at least 4.0 grams, at least 5.0 grams, at least 6.0 grams, at least 7.0 grams, at least 8.0 grams, at least 9.0 grams, or at least 10.0 grams of tissue is used. In one embodiment, the bladder tissue sample is removed without cautery.

In one embodiment, the tissue sample, for example, the bladder tissue sample, is incubated in a cell culture medium. In one embodiment, the cell culture medium is Dulbecco's Modified Eagle Medium (DMEM). In another embodiment, the cell culture medium is Dulbecco's Modified Eagle Medium/Nutrient Mixture F-12 (DMEM/F-12). In one embodiment, the cell culture medium is hepatocyte medium. In another embodiment, the cell culture medium is supplemented with serum. In one embodiment, the cell culture medium is supplemented with fetal bovine serum (FBS). In another embodiment, the cell culture medium is supplemented with 5% fetal bovine serum (FBS). In one embodiment, the cell culture medium is supplemented with about 0.1% FBS, about 0.2% FBS, about 0.3% FBS, about 0.4% FBS, about 0.5% FBS, about 0.6% FBS, about 0.7% FBS, about 0.8% FBS, about 0.9% FBS, about 1% FBS, about 2% FBS, about 3% FBS, about 4% FBS, about 5% FBS, about 6% FBS, about 7% FBS, about 8% FBS, about 9% FBS, about 10% FBS, about 15% FBS, or about 20% FBS, or more.

In one embodiment, the cell culture medium is supplemented with at least 0.1% FBS, with at least 0.2% FBS, with at least 0.3% FBS, with at least 0.4% FBS, with at least 0.5% FBS, with at least 0.6% FBS, with at least 0.7% FBS, with at least 0.8% FBS, with at least 0.9% FBS, with at least 1% FBS, with at least 2% FBS, with at least 3% FBS, with at least 4% FBS, with at least 5% FBS, with at least 6% FBS, with at least 7% FBS, with at least 8% FBS, with at least 9% FBS, with at least 10% FBS, or with at least 20% FBS.

In one embodiment, the tissue sample, for example, the bladder tissue sample, is dissociated into a single cell suspension. In another embodiment, the tissue sample, for example, the bladder tissue sample, is dissociated into cell clusters. In one embodiment, cell clusters comprise about 5 to 50 cells. In another embodiment, cell clusters comprise about 5, about 10, about 15, about 20, about 25, about 30, about 35, about 40, about 45, about 50, about 55, about 60, about 65, about 70, about 75, about 80, about 85, about 90, about 95, or about 100 cells.

In one embodiment, the tissue sample, for example, the bladder tissue sample, is dissociated mechanically. In one embodiment, the tissue sample is dissociated mechanically by mincing with scissors.

In one embodiment, the tissue sample, for example, the bladder tissue sample, is dissociated enzymatically. In one embodiment, the tissue sample is dissociated enzymatically by incubation of tissue with cell culture medium supplemented with collagenase. Collagenase can break down the collagen found in tissues. In one embodiment, the final concentration of collagenase in the cell culture medium is 300 units/ml. In another embodiment, the final concentration of collagenase in the cell culture medium is at least 50 units/ml, at least 100 units/ml, at least 200 units/ml, at least 300 units/ml, at least 400 units/ml, at least 500 units/ml, at least 600 units/ml, at least 700 units/ml, at least 800 units/ml, at least 900 units/ml, or at least 1000 units/ml.

In one embodiment, the tissue sample, for example, the bladder tissue sample, is dissociated enzymatically by incubation of the tissue with cell culture medium supplemented with hyaluronidase. Hyaluronidase can break down the hyaluronic acid found in tissues. In one embodiment, the final concentration of hyaluronidase in the cell culture medium is 100 units/ml. In another embodiment, the final concentration of hyaluronidase in the cell culture medium is at least 10 units/ml, at least 20 units/ml, at least 30 units/ml, at least 40 units/ml, at least 50 units/ml, at least 60 units/ml, at least 70 units/ml, at least 80 units/ml, at least 90 units/ml, at least 100 units/ml, at least 200 units/ml, at least 300 units/ml, at least 400 units/ml, at least 500 units/ml, at least 600 units/ml, at least 700 units/ml, at least 800 units/ml, at least 900 units/ml, or at least 1000 units/ml.

In one embodiment, the cell culture medium is supplemented with both collagenase and hyaluronidase. In another embodiment, a 10× concentrated solution of collagenase and hyaluronidase is diluted 10-fold in the cell culture medium.

In one embodiment, the tissue sample, for example, the bladder tissue sample, is incubated in DMEM/F12 with 5% FBS, 300 units/ml collagenase, and 100 units/ml hyaluronidase for 3 hours at 37° C. In one embodiment, the sample is incubated for at least 1 hours, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In one embodiment, the tissue sample, for example, the bladder tissue sample, is incubated in hepatocyte medium with 5% FBS, 300 units/ml collagenase, and 100 units/ml hyaluronidase for 1 hours at 37° C. In one embodiment, the sample is incubated for at least 1 hours, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In one embodiment, dissociated tissue, for example, dissociated bladder tissue, is separated from the dissociating medium by centrifugation. In one embodiment, the tissue can be further dissociated by incubation of the tissue with Accutase™. Accutase™ is a cell detachment solution of proteolytic and collagenolytic enzymes. In one embodiment, the bladder tissue is added to a 1× Accutase™ Solution. In one embodiment, the tissue can be further dissociated by incubation of the tissue with TrypLE™. TrypLE™ is an animal origin-free recombinant enzyme alternative to porcine or bovine trypsin. TrypLE™ cleaves peptide bonds on the C-terminal side of lysine and arginine. In one embodiment, the tissue can be further dissociated by incubation of the tissue with trypsin. In one embodiment, the sample is incubated for 30 minutes at 37° C. In one embodiment, the sample is incubated for 20 minutes at 37° C. In one embodiment, the sample is incubated for at least 5 minutes, at least 10 minutes, at least 15 minutes, at least 20 minutes, at least 30 minutes, at least 45 minutes, at least 1 hour, at least 2 hours, at least 3 hours, at least 4 hours, or at least 5 hours. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, Accutase™ TrypLE™, or trypsin activity is stopped by the addition of HBSS containing 2% FBS. In one embodiment, the HBSS does not contain Ca²⁺. In another embodiment, the HBSS does not contain Mg′. In one embodiment, the HBSS contains Ca²⁺. In another embodiment, the HBSS contains Mg²⁺. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, dissociated tissue, for example, dissociated bladder tissue, is separated from the Accutase™, TrypLE™, or trypsin solution by centrifugation. In one embodiment, the tissue can be further dissociated by incubation of tissue with dispase. Dispase is a protease and can hydrolyse proteins. In one embodiment, the dispase is dispase II. In one embodiment, the dispase is added to the tissue at a final concentration of 5 mg/ml. In another embodiment, the final concentration of dispase is at least 0.5 mg/ml, at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, at least 10 mg/ml, at least 11 mg/ml, at least 12 mg/ml, at least 13 mg/ml, at least 14 mg/ml, at least 15 mg/ml, at least 16 mg/ml, at least 17 mg/ml, at least 18 mg/ml, at least 19 mg/ml, or at least 20 mg/ml. In one embodiment, dispase is added in Hanks' Balanced Salt Solution (HBSS). In one embodiment, the dispase solution is supplemented with DNase I at a final concentration of 0.1 mg/ml. In another embodiment, the final concentration of DNase I is at least 0.1 mg/ml, at least 0.2 mg/ml, at least 0.3 mg/ml, at least 0.4 mg/ml, at least 0.5 mg/ml units/ml, at least 0.6 mg/ml, at least 0.7 mg/ml, at least 0.8 mg/ml, at least 0.9 mg/ml, at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, or at least 10 mg/ml. In one embodiment, the sample is incubated in dispase supplemented with DNase I for 1 minute with rigorous pipetting. In one embodiment, the sample is incubated for at least 30 seconds, at least 1 minute, at least 2 minutes, at least 3 minutes, at least 4 minutes, or at least 5 minutes. In one embodiment, dispase activity is stopped by the addition of HBSS containing 2% FBS. In one embodiment, the HBSS does not contain Ca²⁺. In another embodiment, the HBSS does not contain Mg²⁺. In one embodiment, the HBSS contains Ca²⁺. In another embodiment, the HBSS contains Mg²⁺. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, the dissociated tissue cell suspension, for example, the dissociated bladder tissue cell suspension is filtered through a 40 μm cell strainer. In one embodiment, the dissociated tissue cell suspension is filtered through a 70 μm cell strainer. In another embodiment, the dissociated tissue cell suspension is filtered through a 100 μm cell strainer.

In one embodiment, the dissociated tissue cell suspension is treated with DNase I. In one embodiment, the dissociated tissue cell suspension is treated with DNase I in hepatocyte medium. In one embodiment, the final concentration of DNase I is 0.1 mg/ml. In another embodiment, the final concentration of DNase I is at least 0.1 mg/ml, at least 0.2 mg/ml, at least 0.3 mg/ml, at least 0.4 mg/ml, at least 0.5 mg/ml units/ml, at least 0.6 mg/ml, at least 0.7 mg/ml, at least 0.8 mg/ml, at least 0.9 mg/ml, at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, or at least 10 mg/ml. In one embodiment, the sample is incubated in DNase I for 5 minutes. In one embodiment, the sample is incubated for at least 30 seconds, at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, or at least 15 minutes.

In one embodiment, cells are dissociated from the tissue, for example, bladder tissue, and subsequently separated, enriched, isolated or purified. The methods comprise obtaining a tissue sample or mixed population of cells, contacting the population of cells with an agent that binds to epithelial cells, for example EpCAM, and separating the subpopulation of cells that are bound by the agent from the subpopulation of cells that are not bound by the agent, wherein the subpopulation that are bound by the agent is enriched for the epithelial marker (for example, EpCAM positive cells). The methods described herein can be performed using any epithelial marker known in the art, including but not limited to CD44R, CD66a, CD75, CD104, CD167, cytokeratin, EpCAM (CD326), CD138, or E-cadherin.

In one embodiment, epithelial cells, for example, bladder epithelial cells, are separated using the EasySep™ Human EpCAM Positive Selection Kit (Stemcell Technologies). Bladder epithelial cells are specifically labeled with dextran-coated magnetic nanoparticles using bispecific Tetrameric Antibody Complexes. These complexes recognize both dextran and the cell surface antigen expressed on the cell. The small size of the magnetic dextran iron particles allows for efficient binding to the TAC-labeled cells. Magnetically labeled cells are then separated from unlabeled cells using the EasySep® procedure.

In one embodiment, epithelial cells, for example, bladder epithelial cells, are separated using a fluorescently-tagged EpCAM antibody.

The methods for separating, enriching, isolating or purifying stem cells from a mixed population of cells according to the invention may be combined with other methods for separating, enriching, isolating or purifying stem or progenitor cells, or epithelial cells, that are known in the art. For example, the methods described herein may be performed in conjunction with techniques that use other epithelial cell markers. For example, an additional selection step may be performed either before, after, or simultaneously with the epithelial cell selection step, in which a second agent, such as an antibody, that binds to a second marker is used. The mixed population of cells can be any source of cells from which to obtain epithelial cells, including but not limited to a tissue biopsy from a subject, a dissociated cell suspension derived from a tissue biopsy, or a population of cells that have been grown in culture.

In one embodiment, the agent used can be any agent that binds to epithelial cells, for example, bladder epithelial cells, as described above. The term “agent” includes, but is not limited to, small molecule drugs, peptides, proteins, peptidomimetic molecules, and antibodies. It also includes any epithelial cell binding molecule that is labeled with a detectable moiety, such as a histological stain, an enzyme substrate, a fluorescent moiety, a magnetic moiety or a radio-labeled moiety. Such “labeled” agents are particularly useful for embodiments involving isolation or purification of bladder epithelial cells, or detection of bladder epithelials cells. In some embodiments, the agent is an antibody that binds to bladder epithelial cells.

There are many cell separation techniques known in the art (U.S. Pat. No. 4,777,145, U.S. Pat. No. 8,004,661, U.S. Pat. No. 5,367,474, U.S. Pat. No. 4,347,935), and any such technique may be used. For example magnetic cell separation techniques can be used if the agent is labeled or bound to an iron-containing moiety or iron particle. In one embodiment, cells may also be passed over a solid support that has been conjugated to an agent that binds to epithelial cells, for example, bladder epithelial cells, such that the epithelial cells will be selectively retained on the solid support. Cells may also be separated by density gradient methods, particularly if the agent selected significantly increases the density of the epithelial cells to which it binds. For example, the agent can be a fluorescently labeled antibody against bladder epithelial cells, and the bladder epithelial cells are separated from the other cells using fluorescence activated cell sorting (FACS).

The methods for separating, enriching, isolating or purifying epithelial cells from a mixed population of cells according to the invention may be combined with other methods for separating, enriching, isolating or purifying cells that are known in the art (for example, U.S. Pat. No. 4,777,145, U.S. Pat. No. 8,004,661, U.S. Pat. No. 5,367,474, U.S. Pat. No. 4,347,935) and are described in P. T. Sharpe, 1988, Laboratory Techniques in Biochemistry and Molecular Biology Volume 18: Methods of Cell Separation, Elsevier, Amsterdam; M. Zborowski and J. J. Chalmers, 2007, Laboratory Techniques in Biochemistry and Molecular Biology Volume 32: Magnetic Cell Separation, Elsevier, Amsterdam; and T. S. Hawley and R. G. Hawley, 2005, Methods in Molecular Biology Volume 263: Flow Cytometry Protocols, Humana Press Inc, Totowa, N.J. For example, the methods described herein may be performed in conjunction with techniques that use other markers. For example, additional selection steps maybe performed either before, after, or simultaneously with the epithelial marker selection step, in which a second agent, such as an antibody, that binds to a second marker is used, separating the subpopulation of cells that are bound by the agent from the subpopulation that are not bound by the agent, wherein the subpopulation of cells that are not bound by the agent is enriched. The second marker may be any marker known in the art that reduces the heterogeneity of the epithelial population. For example, the second marker is a marker for epithelial cells (for example, CD44R, CD66a, CD75, CD104, CD167, cytokeratin, EpCAM (CD326), CD138, or E-cadherin). In another embodiment, the second marker is a combination of any markers known in the art that reduce the heterogeneity of the epithelial population.

Isolated cells can be analyzed by any number of methods. The nucleic acids and/or polypeptides of the isolated cells can be analyzed and quantified by any of a number of general means well known to those of skill in the art. These include, for example, analytical biochemical methods such as radiography, electrophoresis, NMR, spectrophotometry, capillary electrophoresis, thin layer chromatography (TLC), high performance liquid chromatography (HPLC), and hyperdiffusion chromatography; various immunological methods, such as immuno-electrophoresis, Southern analysis, Northern analysis, dot-blot analysis, fluid or gel precipitation reactions, immunodiffusion, quadrature radioimmunoassay (RIAs), enzyme-linked immunosorbent assays (ELISAs), immunofluorescent assays, gel electrophoresis (e.g., SDS-PAGE), nucleic acid or target or signal amplification methods, radiolabeling, scintillation counting, and affinity chromatography.

### Methods of Culturing Bladder Cell Lines and Culture Media

Various culturing parameters can be used with respect to the cell being cultured. Appropriate culture conditions for mammalian cells are well known in the art or can be determined by the skilled artisan (see, for example, Animal Cell Culture: A Practical Approach 2^{nd} Ed., Rickwood, D. and Hames, B. D., eds. (Oxford University Press: New York, 1992)), and vary according to the particular cell selected. Commercially available medium can be utilized. Non-limiting examples of medium include, for example, Dulbecco's Modified Eagle Medium (DMEM, Life Technologies), Dulbecco's Modified Eagle Medium/Nutrient Mixture F-12 (DMEM/F-12, Life Technologies), Minimal Essential Medium (MEM, Sigma, St. Louis, Mo.), and hepatocyte medium.

The media described above can be supplemented as necessary with supplementary components or ingredients, including optional components, in appropriate concentrations or amounts, as necessary or desired. Cell medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, epidermal growth factor and fibroblast growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example, pluronic polyol; and (8) galactose.

The mammalian cell culture that can be used with the present invention is prepared in a medium suitable for the particular cell being cultured. In one embodiment, the culture medium can be one of the aforementioned (for example, DMEM, or basal hepatocyte medium) that is supplemented with serum from a mammalian source (for example, fetal bovine serum (FBS)). For example, Hepatocyte Medium supplemented with FBS can be used to sustain the growth of epithelial cells. In another embodiment, the medium can be DMEM.

Cells maintained in culture can be passaged by their transfer from a previous culture to a culture with fresh medium. In one embodiment, induced epithelial cells are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

The cells suitable for culturing according to the methods of the present invention can harbor introduced expression vectors (constructs), such as plasmids and the like. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

In another aspect, the invention provides a cell culture medium comprising a basal hepatocyte medium, Matrigel, FBS and ROCK inhibitor. In one embodiment, the medium comprises 5% Matrigel. In another embodiment, the medium comprises 5% heat-inactivated charcoal-stripped FBS. In a further embodiment, the medium is used to culture bladder cell lines. In one embodiment, the bladder cell lines are normal. In another embodiment, the bladder cell lines are non-cancerous. In a further embodiment, the bladder cell lines are cancerous.

In one embodiment, the culture medium comprises EGF. In another embodiment, the culture medium does not comprise EGF. In one embodiment, the culture medium comprises serum, including, but not limited to, FBS. In another embodiment, the culture medium does not comprise serum, including, but not limited to, FBS. In one embodiment, the culture medium comprises a ROCK inhibitor. In another embodiment, the culture medium does not comprise a ROCK inhibitor. In one embodiment, the culture medium comprises Matrigel. In another embodiment, the culture medium does not comprise Matrigel.

In one embodiment, epithelial cells, for example, bladder epithelial cells, can be cultured to generate bladder cell lines. In one embodiment, epithelial cells are suspended in hepatocyte medium. In one embodiment, the hepatocyte culture medium is supplemented with 10 ng/ml of EGF. In one embodiment, the hepatocyte culture medium is supplemented with about 1 ng/ml of EGF, 2 ng/ml of EGF, 3 ng/ml of EGF, 4 ng/ml of EGF, 5 ng/ml of EGF, 6 ng/ml of EGF, 7 ng/ml of EGF, 8 ng/ml of EGF, 9 ng/ml of EGF, 10 ng/ml of EGF, 11 ng/ml of EGF, 12 ng/ml of EGF, 13 ng/ml of EGF, 14 ng/ml of EGF, 15 ng/ml of EGF, 16 ng/ml of EGF, 17 ng/ml of EGF, 18 ng/ml of EGF, 19 ng/ml of EGF, about 20 ng/ml of EGF, about 25 ng/ml of EGF, about 30 ng/ml of EGF, about 35 ng/ml of EGF, about 40 ng/ml of EGF, about 45 ng/ml of EGF, about 50 ng/ml of EGF, or more.

In another embodiment, the hepatocyte culture medium is supplemented with at least 1 ng/ml of EGF, at least 2 ng/ml of EGF, at least 3 ng/ml of EGF, at least 4 ng/ml of EGF, at least 5 ng/ml of EGF, at least 6 ng/ml of EGF, at least 7 ng/ml of EGF, at least 8 ng/ml of EGF, at least 9 ng/ml of EGF, at least 10 ng/ml of EGF, at least 15 ng/ml of EGF, at least 20 ng/ml of EGF, at least 30 ng/ml of EGF, at least 40 ng/ml of EGF, or at least 50 ng/ml of EGF.

In one embodiment, the hepatocyte culture medium is supplemented with 2 mM of GlutaMAX™. GlutaMAX™ is the dipeptide L-alanyl-L-glutamine. In one embodiment, the hepatocyte culture medium is supplemented with at least 0.1 mM of GlutaMAX™, at least 0.5 mM of GlutaMAX™, at least 1 mM of GlutaMAX™, at least 1.5 mM of GlutaMAX™, at least 2 mM of GlutaMAX™, at least 3 mM of GlutaMAX™, at least 4 mM of GlutaMAX™, or at least 5 mM of GlutaMAX™. In another embodiment, the hepatocyte culture medium is supplemented with L-glutamine.

In one embodiment, the hepatocyte culture medium is supplemented with 5% Matrigel™. In one embodiment, the hepatocyte culture medium is supplemented with about 0.1% Matrigel™, about 0.2% Matrigel™, about 0.3% Matrigel™, about 0.4% Matrigel™ about 0.5% Matrigel™, about 0.6% Matrigel™, about 0.7% Matrigel™, about 0.8% Matrigel™, about 0.9% Matrigel™, about 1% Matrigel™, about 2% Matrigel™, about 3% Matrigel™, about 4% Matrigel™, about 5% Matrigel™, about 6% Matrigel™, about 7% Matrigel™, about 8% Matrigel™, about 9% Matrigel™, about 10% Matrigel™, about 15% Matrigel™, or about 20% Matrigel™.

In one embodiment, the hepatocyte culture medium is supplemented with at least 0.1% Matrigel™, at least 0.2% Matrigel™, at least 0.3% Matrigel™, at least 0.4% Matrigel™, at least 0.5% Matrigel™, at least 0.6% Matrigel™, at least 0.7% Matrigel™, at least 0.8% Matrigel™, at least 0.9% Matrigel™, at least 1% Matrigel™, at least 2% Matrigel™, at least 3% Matrigel™, at least 4% Matrigel™, at least 5% Matrigel™, at least 6% Matrigel™, at least 7% Matrigel™, at least 8% Matrigel™, at least 9% Matrigel™, at least 10% Matrigel™, or at least 20% Matrigel™.

In one embodiment, the hepatocyte culture medium is supplemented with 5% FBS. In another embodiment, the FBS is heat-inactivated charcoal-stripped FBS. In one embodiment, the hepatocyte culture medium is supplemented with about 0.1% FBS, about 0.2% FBS, about 0.3% FBS, about 0.4% FBS, about 0.5% FBS, about 0.6% FBS, about 0.7% FBS, about 0.8% FBS, about 0.9% FBS, about 1% FBS, about 2% FBS, about 3% FBS, about 4% FBS, about 5% FBS, about 6% FBS, about 7% FBS, about 8% FBS, about 9% FBS, about 10% FBS, about 15% FBS, or about 20% FBS, or more.

In one embodiment, the hepatocyte culture medium is supplemented with at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, the hepatocyte culture medium is supplemented with a Rho-Associated Coil Kinase (ROCK) inhibitor. In one embodiment, the ROCK inhibitor is Y-27632. In one embodiment, the hepatocyte culture medium is supplemented with 1004 of Y-27632. In another embodiment, the hepatocyte culture medium is supplemented with about 1 μM of Y-27632, about 2 μM of Y-27632, about 3 μM of Y-27632, about 4 μM of Y-27632, about 5 μM of Y-27632, about 6 μM of Y-27632, about 7 μM of Y-27632, about 8 μM of Y-27632, about 9 μM of Y-27632, about 1004 of Y-27632, about 11 μM of Y-27632, about 1204 of Y-27632, about 1304 of Y-27632, about 1404 of Y-27632, about 15 μM of Y-27632, about 2004 of Y-27632, about 3004 of Y-27632, about 4004 of Y-27632, or about 5004 of Y-27632, or more.

In another embodiment, the hepatocyte culture medium is supplemented with at least 1 μM of Y-27632, at least 2 μM of Y-27632, at least 3 μM of Y-27632, at least 4 μM of Y-27632, at least 5 μM of Y-27632, at least 6 μM of Y-27632, at least 7 μM of Y-27632, at least 8 μM of Y-27632, at least 9 μM of Y-27632, at least 1004 of Y-27632, at least 11 μM of Y-27632, at least 1204 of Y-27632, at least 1304 of Y-27632, at least 1404 of Y-27632, at least 1504 of Y-27632, at least 2004 of Y-27632, at least 3004 of Y-27632, at least 4004 of Y-27632, or at least 5004 of Y-27632.

In one embodiment, the epithelial cells, for example, bladder epithelial cells, are plated into wells of a tissue culture plate. In another embodiment, the epithelial cells are plated into wells of a Primaria™ 24 well flat bottom surface modified multiwell cell culture plate. In another embodiment, the bladder epithelial cells are plated in wells of a plate that enhances or maximizes attachment of the cells to the wells. In another embodiment, the plate is a polystyrene plate. In a further embodiment, the plate is a surface modified polystyrene plate. Without being bound by theory, the surface of the plate can be modified to incorporate anionic and cationic functional groups to enhance the attachment of the cells to the surface if the plate.

In one embodiment, the epithelial cells, for example, bladder epithelial cells, are plated into wells of a 24 well plate at a final density of 75,000 cells per well. In another embodiment, the cells are plated into wells of a 24 well plate at a final density of about 50,000 cells per well, about 55,000 cells per well, about 60,000 cells per well, about 65,000 cells per well, about 70,000 cells per well, about 75,000 cells per well, about 80,000 cells per well, about 85,000 cells per well, about 90,000 cells per well, about 95,000 cells per well, or about 100,000 cells per well. Without being bound by theory, a well of a 24 well plate has a surface area of about 1.9 cm².

In another embodiment, cells are plated into wells of a 24 well plate at a final density of at least 50,000 cells per well, at least 55,000 cells per well, at least 60,000 cells per well, at least 65,000 cells per well, at least 70,000 cells per well, at least 75,000 cells per well, at least 80,000 cells per well, at least 85,000 cells per well, at least 90,000 cells per well, at least 95,000 cells per well, or at least 100,000 cells per well.

In one embodiment, a total change of media occurs every 3 days. In one embodiment, a total change of media occurs every 4 days. In another embodiment, a total change of media occurs at least every day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, or at least every 14 days.

In one embodiment, the bladder epithelial cells form bladder cell line colonies. In one embodiment when the bladder cell lines have reached about 75% confluence the cells are passaged. In another embodiment, when the bladder cell lines have reached about 60%, about 65%, about 70%, about 75%, about 80%, about 85%, about 90%, about 95%, or about 99% confluence the cells are passaged.

Cells can be passaged by their transfer from a previous culture to a culture with fresh medium. In one embodiment, induced epithelial cells are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

In one embodiment, the cells, for example, the bladder cell lines, are prepared for passaging by addition of Dispase to each well. In one embodiment, the Dispase is added at a final concentration of 1 mg/ml for 10 minutes at 37° C. In another embodiment, the final concentration of dispase is at least 0.2 mg/ml, at least 0.3 mg/ml, at least 0.4 mg/ml, at least 0.5 mg/ml, at least 0.6 mg/ml, at least 0.7 mg/ml, at least 0.8 mg/ml, at least 0.9 mg/ml, at least 1.0 mg/ml, at least 1.5 mg/ml, at least 2.0 mg/ml, at least 2.5 mg/ml, or at least 3 mg/ml. In one embodiment, the cells are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, or at least 20 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, the dispase solution is discarded and residual Matrigel is removed with cold PBS.

In one embodiment, the cells, for example, the bladder cell lines, are passaged by addition of Accutase™ to each well. In one embodiment, the Accutase™ is added for 15 minutes at 37° C. In one embodiment, the cells are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 25 minutes, or at least 30 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment the Accutase™ activity is stopped by the addition of HBSS containing 2% FBS. In one embodiment, the HBSS does not contain Ca²⁺. In another embodiment, the HBSS does not contain Mg′. In one embodiment, the HBSS contains Ca²⁺. In another embodiment, the HBSS contains Mg²⁺. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, detached cells, for example, detached bladder cells, are separated from the Accutase™ containing medium by centrifugation. In one embodiment, the cells are plated into a new Primaria™ 24 well flat bottom surface modified multiwell cell culture plate. In one embodiment, the cells are plated into a new 96 well low attachment plate. Without being bound by theory, bladder cell lines can be converted to organoids. In one embodiment, the cells are plated as described for the Matrigel floating method. In one embodiment, the cells are plated as described for the Matrigel embedding method. In one embodiment, the cells are plated by the collagen embedding method.

In one embodiment, detached cells, for example, detached bladder cells, are separated from the Accutase™ containing medium by centrifugation. In one embodiment, the cells are frozen by resuspending the detached cells in a freezing media. In one embodiment, the freezing media comprises hepatocyte medium, FBS, and DMSO. In one embodiment, the freezing media contains about 50% FBS, about 40% hepatocyte media, and about 10% DMSO. In one embodiment, the FBS is heat-inactivated charcoal-stripped FBS. In one embodiment, cells are gradually frozen to less than or equal to −80° C.

In one embodiment, frozen cells, for example, frozen bladder cell lines, can be thawed. In one embodiment, the frozen cells are thawed rapidly in at about 37° C. and immediately diluted in HBSS containing 2% FBS. In one embodiment, the thawed cells are immediately separated from the freezing media by centrifugation. In one embodiment, the cells are plated into a new Primaria™ 24 well flat bottom surface modified multiwell cell culture plate. In one embodiment, the cells are plated into a new 96 well low attachment plate. Without being bound by theory, bladder cell lines can be converted to organoids. In one embodiment, the cells are plated as described for the Matrigel floating method. In one embodiment, the cells are plated as described for the Matrigel embedding method. In one embodiment, the cells are plated by the collagen embedding method.

### Methods of Culturing Organoids and Culture Media

Various culturing parameters can be used with respect to the cell or organoid being cultured. Appropriate culture conditions for mammalian cells or organoids are well known in the art or can be determined by the skilled artisan (see, for example, Animal Cell Culture: A Practical Approach 2nd Ed., Rickwood, D. and Hames, B. D., eds. (Oxford University Press: New York, 1992)), and vary according to the particular cell or organoid selected. Commercially available medium can be utilized. Non-limiting examples of medium include, for example, Dulbecco's Modified Eagle Medium (DMEM, Life Technologies), Dulbecco's Modified Eagle Medium/Nutrient Mixture F-12 (DMEM/F-12, Life Technologies), Minimal Essential Medium (MEM, Sigma, St. Louis, Mo.), and hepatocyte medium.

The media described above can be supplemented as necessary with supplementary components or ingredients, including optional components, in appropriate concentrations or amounts, as necessary or desired. Cell or organoid medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, epidermal growth factor and fibroblast growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example, pluronic polyol; and (8) galactose.

The mammalian cell or organoid culture that can be used with the present invention is prepared in a medium suitable for the particular cell or organoid being cultured. In one embodiment, the culture medium can be one of the aforementioned (for example, DMEM, or basal hepatocyte medium) that is supplemented with serum from a mammalian source (for example, fetal bovine serum (FBS)). For example, Hepatocyte Medium supplemented with FBS can be used to sustain the growth of epithelial cells or organoids. In another embodiment, the medium can be DMEM.

Cells or organoids maintained in culture can be passaged by their transfer from a previous culture to a culture with fresh medium. In one embodiment, induced epithelial cells or organoids are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

The cells suitable for culturing according to the methods of the present invention can harbor introduced expression vectors (constructs), such as plasmids and the like. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

In another aspect, the invention provides a cell culture medium comprising a basal hepatocyte medium, Matrigel, FBS and ROCK inhibitor. In one embodiment, the medium comprises 5% Matrigel. In another embodiment, the medium comprises 5% heat-inactivated charcoal-stripped FBS. In a further embodiment, the medium is used to culture bladder organoids. In one embodiment, the bladder organoids are normal. In another embodiment, the bladder organoids are non-cancerous. In a further embodiment, the bladder organoids are cancerous.

In another aspect, the invention provides a cell culture medium comprising a basal hepatocyte medium and FBS. In one embodiment, the medium comprises 5% heat-inactivated charcoal-stripped FBS. In a further embodiment, the medium is used to culture bladder organoids. In one embodiment, the bladder organoids are normal. In another embodiment, the bladder organoids are non-cancerous. In a further embodiment, the bladder organoids are cancerous.

In one embodiment, the culture medium comprises EGF. In another embodiment, the culture medium does not comprise EGF. In one embodiment, the culture medium comprises serum, including, but not limited to, FBS. In another embodiment, the culture medium does not comprise serum, including, but not limited to, FBS. In one embodiment, the culture medium comprises a ROCK inhibitor. In another embodiment, the culture medium does not comprise a ROCK inhibitor. In one embodiment, the culture medium comprises Matrigel. In another embodiment, the culture medium does not comprise Matrigel. In one embodiment, the culture medium comprises Glutamax. In another embodiment, the culture medium does not comprise Glutamax.

In one embodiment, epithelial cells, for example, bladder epithelial cells, can be cultured to generate bladder organoids. In one embodiment, epithelial cells are suspended in hepatocyte medium. In one embodiment, epithelial cells are suspended in a Matrigel matrix and overlaid with a hepatocyte medium. In another embodiment, epithelial cells are suspended in a collagen matrix and overlaid with a medium. In one embodiment, the hepatocyte culture medium is supplemented with 10 ng/ml of EGF. In one embodiment, the hepatocyte culture medium is supplemented with about 1 ng/ml of EGF, 2 ng/ml of EGF, 3 ng/ml of EGF, 4 ng/ml of EGF, 5 ng/ml of EGF, 6 ng/ml of EGF, 7 ng/ml of EGF, 8 ng/ml of EGF, 9 ng/ml of EGF, 10 ng/ml of EGF, 11 ng/ml of EGF, 12 ng/ml of EGF, 13 ng/ml of EGF, 14 ng/ml of EGF, 15 ng/ml of EGF, 16 ng/ml of EGF, 17 ng/ml of EGF, 18 ng/ml of EGF, 19 ng/ml of EGF, about 20 ng/ml of EGF, about 25 ng/ml of EGF, about 30 ng/ml of EGF, about 35 ng/ml of EGF, about 40 ng/ml of EGF, about 45 ng/ml of EGF, about 50 ng/ml of EGF, or more.

In another embodiment, the hepatocyte culture medium is supplemented with at least 1 ng/ml of EGF, at least 2 ng/ml of EGF, at least 3 ng/ml of EGF, at least 4 ng/ml of EGF, at least 5 ng/ml of EGF, at least 6 ng/ml of EGF, at least 7 ng/ml of EGF, at least 8 ng/ml of EGF, at least 9 ng/ml of EGF, at least 10 ng/ml of EGF, at least 15 ng/ml of EGF, at least 20 ng/ml of EGF, at least 30 ng/ml of EGF, at least 40 ng/ml of EGF, or at least 50 ng/ml of EGF.

In one embodiment, the hepatocyte culture medium is supplemented with 2 mM of GlutaMAX™. GlutaMAX™ is the dipeptide L-alanyl-L-glutamine. In one embodiment, the hepatocyte culture medium is supplemented with at least 0.1 mM of GlutaMAX™, at least 0.5 mM of GlutaMAX™, at least 1 mM of GlutaMAX™, at least 1.5 mM of GlutaMAX™, at least 2 mM of GlutaMAX™, at least 3 mM of GlutaMAX™, at least 4 mM of GlutaMAX™, or at least 5 mM of GlutaMAX™. In another embodiment, the hepatocyte culture medium is supplemented with L-glutamine.

In one embodiment, the hepatocyte culture medium is supplemented with 5% Matrigel™. In one embodiment, the hepatocyte culture medium is supplemented with about 0.1% Matrigel™, about 0.2% Matrigel™, about 0.3% Matrigel™, about 0.4% Matrigel™ about 0.5% Matrigel™, about 0.6% Matrigel™, about 0.7% Matrigel™, about 0.8% Matrigel™, about 0.9% Matrigel™, about 1% Matrigel™, about 2% Matrigel™, about 3% Matrigel™, about 4% Matrigel™, about 5% Matrigel™, about 6% Matrigel™, about 7% Matrigel™, about 8% Matrigel™, about 9% Matrigel™, about 10% Matrigel™, about 15% Matrigel™, or about 20% Matrigel™.

In one embodiment, the hepatocyte culture medium is supplemented with at least 0.1% Matrigel™, at least 0.2% Matrigel™, at least 0.3% Matrigel™, at least 0.4% Matrigel™, at least 0.5% Matrigel™, at least 0.6% Matrigel™, at least 0.7% Matrigel™, at least 0.8% Matrigel™, at least 0.9% Matrigel™, at least 1% Matrigel™, at least 2% Matrigel™, at least 3% Matrigel™, at least 4% Matrigel™, at least 5% Matrigel™, at least 6% Matrigel™, at least 7% Matrigel™, at least 8% Matrigel™, at least 9% Matrigel™, at least 10% Matrigel™, or at least 20% Matrigel™.

In one embodiment, the hepatocyte culture medium is supplemented with 5% FBS. In another embodiment, the FBS is heat-inactivated charcoal-stripped FBS. In one embodiment, the hepatocyte culture medium is supplemented with about 0.1% FBS, about 0.2% FBS, about 0.3% FBS, about 0.4% FBS, about 0.5% FBS, about 0.6% FBS, about 0.7% FBS, about 0.8% FBS, about 0.9% FBS, about 1% FBS, about 2% FBS, about 3% FBS, about 4% FBS, about 5% FBS, about 6% FBS, about 7% FBS, about 8% FBS, about 9% FBS, about 10% FBS, about 15% FBS, or about 20% FBS, or more.

In one embodiment, the hepatocyte culture medium is supplemented with at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, the hepatocyte culture medium is supplemented with a Rho-Associated Coil Kinase (ROCK) inhibitor. In one embodiment, the ROCK inhibitor is Y-27632. In one embodiment, the hepatocyte culture medium is supplemented with 1004 of Y-27632. In another embodiment, the hepatocyte culture medium is supplemented with about 1 μM of Y-27632, about 2 μM of Y-27632, about 3 μM of Y-27632, about 4 μM of Y-27632, about 5 μM of Y-27632, about 6 μM of Y-27632, about 7 μM of Y-27632, about 8 μM of Y-27632, about 9 μM of Y-27632, about 1004 of Y-27632, about 1104 of Y-27632, about 1204 of Y-27632, about 1304 of Y-27632, about 1404 of Y-27632, about 1504 of Y-27632, about 2004 of Y-27632, about 3004 of Y-27632, about 4004 of Y-27632, or about 5004 of Y-27632, or more.

In another embodiment, the hepatocyte culture medium is supplemented with at least 1 μM of Y-27632, at least 2 μM of Y-27632, at least 3 μM of Y-27632, at least 4 μM of Y-27632, at least 5 μM of Y-27632, at least 6 μM of Y-27632, at least 7 μM of Y-27632, at least 8 μM of Y-27632, at least 9 μM of Y-27632, at least 1004 of Y-27632, at least 11 μM of Y-27632, at least 1204 of Y-27632, at least 1304 of Y-27632, at least 1404 of Y-27632, at least 1504 of Y-27632, at least 2004 of Y-27632, at least 3004 of Y-27632, at least 4004 of Y-27632, or at least 5004 of Y-27632.

In one embodiment, the epithelial cells, for example, bladder epithelial cells, are plated into wells of a tissue culture plate. In another embodiment, the epithelial cells are plated into wells of a 96-well low attachment cell culture plate. In another embodiment, the bladder epithelial cells are plated in wells of a plate that minimizes the attachment of the cells to the wells. In another embodiment, the plate is a polystyrene plate. In a further embodiment, the plate is a surface modified polystyrene plate. In another embodiment, the surface of the plate is hydrophilic and neutral. Without being bound by theory, the surface of the plate can be modified to the plate has a covalently bonded hydrogel surface to minimize the attachment of the cells to the surface if the plate.

In one embodiment, the epithelial cells, for example, bladder epithelial cells, are plated into wells of a 96 well plate at a final density of 5,000 cells per well. In another embodiment, the cells are plated into wells of a 96 well plate at a final density of about 2,500 cells per well, about 3,000 cells per well, about 3,500 cells per well, about 4,000 cells per well, about 4,500 cells per well, about 5,000 cells per well, about 5,500 cells per well, about 6,000 cells per well, about 6,500 cells per well, about 7,000 cells per well, or about 7,500 cells per well. Without being bound by theory, a well of a 96 well plate has a surface area of about 0.32 cm².

In another embodiment, cells are plated into wells of a 96 well plate at a final density of at least 2,500 cells per well, at least 3,000 cells per well, at least 3,500 cells per well, at least 4,000 cells per well, at least 4,500 cells per well, at least 5,000 cells per well, at least 5,500 cells per well, at least 6,000 cells per well, at least 6,500 cells per well, at least 7,000 cells per well, or at least 5 cells per well.

In one embodiment, the epithelial cells, for example, bladder epithelial cells, are contacted with a Matrigel solution that forms a matrix and an overlay layer of liquid culture medium is provided. In one embodiment the Matrigel solution and bladder epithelial cells are plated in a cell culture support. In one embodiment the Matrigel solution and bladder epithelial cells are plated into wells of a tissue culture plate. In another embodiment, the plate is a polystyrene plate. In a further embodiment, the cell culture support is a surface modified polystyrene plate. In one embodiment, the support surface is pre-coated by rinsing Matrigel solution over the support surface and incubating the cell culture support at 37° C. for at least 30 minutes. In one embodiment, the Matrigel solution comprises hepatocyte medium and Matrigel. In one embodiment, the Matrigel solution comprises serum, including, but not limited to, FBS. In another embodiment, the Matrigel solution does not comprise serum, including, but not limited to, FBS. In one embodiment, the Matrigel solution comprises 3 parts Matrigel to 2 parts hepatocyte medium. In one embodiment, the Matrigel solution comprises 60% Matrigel and 40% hepatocyte medium.

In one embodiment, the bladder cell clusters, are contacted with a Matrigel solution that forms a matrix and an overlay layer of liquid culture medium is provided. In one embodiment the Matrigel solution and bladder cell clusters are plated in a cell culture support. In one embodiment the Matrigel solution and bladder cell clusters are plated into wells of a tissue culture plate. In another embodiment, the plate is a polystyrene plate. In a further embodiment, the cell culture support is a surface modified polystyrene plate. In one embodiment, the support surface is pre-coated by rinsing Matrigel solution over the support surface and incubating the cell culture support at 37° C. for at least 30 minutes. In one embodiment, the Matrigel solution comprises hepatocyte medium and Matrigel. In one embodiment, the Matrigel solution comprises serum, including, but not limited to, FBS. In another embodiment, the Matrigel solution does not comprise serum, including, but not limited to, FBS. In one embodiment, the Matrigel solution comprises 3 parts Matrigel to 2 parts hepatocyte medium. In one embodiment, the Matrigel solution comprises 60% Matrigel and 40% hepatocyte medium. In one embodiment, the bladder cell clusters are plated into wells of a 6 well plate, a 12 well plate, a 24 well plate, a 48 well plate, or a 96 well plate.

In one embodiment, the epithelial cells, for example, bladder epithelial cells, are contacted with a collagen solution that forms a matrix and an overlay layer of liquid culture medium is provided. In one embodiment the collagen solution and bladder epithelial cells are plated in a cell culture support. In one embodiment the collagen solution and bladder epithelial cells are plated into wells of a tissue culture plate. In another embodiment, the plate is a polystyrene plate. In a further embodiment, the cell culture support is a surface modified polystyrene plate. In one embodiment, the support surface is pre-coated by rinsing collagen solution over the support surface and incubating the cell culture support at 37° C. for at least 30 minutes. In one embodiment, the collagen solution comprises setting solution and collagen. In one embodiment, the collagen solution comprises 9 parts collagen to 1 parts setting solution. In one embodiment, setting solution comprises EBSS, sodium bicarbonate and sodium hydroxide.

In one embodiment, the bladder cell clusters, are contacted with a collagen solution that forms a matrix and an overlay layer of liquid culture medium is provided. In one embodiment the collagen solution and bladder cell clusters are plated in a cell culture support. In one embodiment the collagen solution and bladder cell clusters are plated into wells of a tissue culture plate. In another embodiment, the plate is a polystyrene plate. In a further embodiment, the cell culture support is a surface modified polystyrene plate. In one embodiment, the support surface is pre-coated by rinsing collagen solution over the support surface and incubating the cell culture support at 37° C. for at least 30 minutes. In one embodiment, the collagen solution comprises setting solution and collagen. In one embodiment, the collagen solution comprises 9 parts collagen to 1 parts setting solution. In one embodiment, setting solution comprises EBSS, sodium bicarbonate and sodium hydroxide.

In one embodiment, the bladder cell clusters are plated into wells of a 6 well plate at a density of 3200 to 8000 cell clusters per well. In one embodiment, the bladder cell clusters are plated into wells of a 6 well plate at a density of about 3000 cell clusters per well, about 3500 cell clusters per well, about 4000 cell clusters per well, about 4500 cell clusters per well, about 5000 cell clusters per well, about 5500 cell clusters per well, about 6000 cell clusters per well, about 6500 cell clusters per well, about 7000 cell clusters per well, about 7500 cell clusters per well, about 8000 cell clusters per well, about 8500 cell clusters per well, about 9000 cell clusters per well, about 9500 cell clusters per well, or about 10000 cell clusters per well.

In one embodiment, the bladder cell clusters are plated into wells of a 6 well plate at a density of at least 3000 cell clusters per well, at least 3500 cell clusters per well, at least 4000 cell clusters per well, at least 4500 cell clusters per well, at least 5000 cell clusters per well, at least 5500 cell clusters per well, at least 6000 cell clusters per well, at least 6500 cell clusters per well, at least 7000 cell clusters per well, at least 7500 cell clusters per well, at least 8000 cell clusters per well, at least 8500 cell clusters per well, at least 9000 cell clusters per well, at least 9500 cell clusters per well, or at least 10,000 cell clusters per well.

In one embodiment, the bladder cell clusters are plated into wells of a 12 well plate at a density of 1600 to 4000 cell clusters per well. In one embodiment, the bladder cell clusters are plated into wells of a 12 well plate at a density of about 1500 cell clusters per well, about 2000 cell clusters per well, about 2500 cell clusters per well, about 3000 cell clusters per well, about 3500 cell clusters per well, about 4000 cell clusters per well, about 4500 cell clusters per well, or about 5000 cell clusters per well.

In one embodiment, the bladder cell clusters are plated into wells of a 12 well plate at a density of at least 1500 cell clusters per well, at least 2000 cell clusters per well, at least 2500 cell clusters per well, at least 3000 cell clusters per well, at least 3500 cell clusters per well, at least 4000 cell clusters per well, at least 4500 cell clusters per well, or at least 5000 cell clusters per well.

In one embodiment, the bladder cell clusters are plated into wells of a 24 well plate at a density of 800 to 2000 cell clusters per well. In one embodiment, the bladder cell clusters are plated into wells of a 24 well plate at a density of about 500 cell clusters per well, about 600 cell clusters per well, about 700 cell clusters per well, about 800 cell clusters per well, about 900 cell clusters per well, about 1000 cell clusters per well, about 1100 cell clusters per well, about 1200 cell clusters per well, about 1300 cell clusters per well, about 1400 cell clusters per well, about 1500 cell clusters per well, about 1600 cell clusters per well, about 1700 cell clusters per well, about 1800 cell clusters per well, about 1900 cell clusters per well, about 2000 cell clusters per well, about 2100 cell clusters per well, about 2200 cell clusters per well, about 2300 cell clusters per well, about 2400 cell clusters per well, or about 2500 cell clusters per well.

In one embodiment, the bladder cell clusters are plated into wells of a 24 well plate at a density of at least 500 cell clusters per well, at least 600 cell clusters per well, at least 700 cell clusters per well, at least 800 cell clusters per well, at least 900 cell clusters per well, at least 1000 cell clusters per well, at least 1100 cell clusters per well, at least 1200 cell clusters per well, at least 1300 cell clusters per well, at least 1400 cell clusters per well, at least 1500 cell clusters per well, at least 1600 cell clusters per well, at least 1700 cell clusters per well, at least 1800 cell clusters per well, at least 1900 cell clusters per well, at least 2000 cell clusters per well, at least 2100 cell clusters per well, at least 2200 cell clusters per well, at least 2300 cell clusters per well, at least 2400 cell clusters per well, or at least 2500 cell clusters per well. In one embodiment, the bladder cell clusters are plated into wells of a 96 well plate at a density of 200 to 500 cell clusters per well. In one embodiment, the bladder cell clusters are plated into wells of a 96 well plate at a density of about 50 cell clusters per well, about 100 cell clusters per well, about 150 cell clusters per well, about 200 cell clusters per well, about 250 cell clusters per well, about 300 cell clusters per well, about 350 cell clusters per well, about 400 cell clusters per well, about 450 cell clusters per well, about 500 cell clusters per well, about 550 cell clusters per well, or about 600 cell clusters per well.

In one embodiment, the bladder cell clusters are plated into wells of a 96 well plate at a density of at least 50 cell clusters per well, at least 100 cell clusters per well, at least 150 cell clusters per well, at least 200 cell clusters per well, at least 250 cell clusters per well, at least 300 cell clusters per well, at least 350 cell clusters per well, at least 400 cell clusters per well, at least 450 cell clusters per well, at least 500 cell clusters per well, at least 550 cell clusters per well, or at least 600 cell clusters per well.

In one embodiment, the bladder epithelial cells form bladder organoids.

In one embodiment, fresh media is added about every 4 days. In another embodiment, a fresh media is added at least every day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, or at least every 14 days. In one embodiment, old media is removed before the addition of fresh media. In one embodiment, organoids are separated from old media by centrifugation, followed by the addition of fresh media to the organoids.

In one embodiment, a total change of media occurs every 3 days. In one embodiment, a total change of media occurs every 4 days. In another embodiment, a total change of media occurs at least every day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, or at least every 14 days.

In one embodiment, when the bladder organoids become large the organoids are passaged. In one embodiment, organoids are passaged 3 to 5 weeks after plating. In another embodiment, organoids are passaged about 1 week after plating, about 2 weeks after plating, about 3 weeks after plating, about 4 weeks after plating, about 5 weeks after plating, about about 6 weeks after plating, or about 7 weeks after plating.

Organoids can be passaged by their transfer from a previous culture to a culture with fresh medium. In one embodiment, induced organoids are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

In one embodiment, the cells, for example, the bladder organoids, are prepared for passaging by separation of the organoids from the media by centrifugation. In one embodiment, organoids can be washed in cold PBS.

In one embodiment, the organoids, for example, the bladder organoids, are passaged by addition of Accutase™ to the organoids. In one embodiment, the Accutase™ is added for 15 minutes at 37° C. In one embodiment, the cells are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 25 minutes, or at least 30 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment the Accutase™ activity is stopped by the addition of HBSS containing 2% FBS. In one embodiment, the HBSS does not contain Ca²⁺. In another embodiment, the HBSS does not contain Mg²⁺. In one embodiment, the HBSS contains Ca²⁺. In another embodiment, the HBSS contains Mg′. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, Accutase™ treated organoids, for example, Accutase™ treated bladder organoids, are separated from the Accutase™ containing medium by centrifugation. In one embodiment, the cells are plated into a new 96-well low attachment cell culture plate. In one embodiment, the dissociated organoid cells, for example, dissociated bladder organoid cells, are plated into wells of a 96 well plate at a final density of 5,000 cells per well. In another embodiment, the cells are plated into wells of a 96 well plate at a final density of about 2,500 cells per well, about 3,000 cells per well, about 3,500 cells per well, about 4,000 cells per well, about 4,500 cells per well, about 5,000 cells per well, about 5,500 cells per well, about 6,000 cells per well, about 6,500 cells per well, about 7,000 cells per well, or about 7,500 cells per well. Without being bound by theory, a well of a 96 well plate has a surface area of about 0.32 cm².

In another embodiment, cells are plated into wells of a 96 well plate at a final density of at least 2,500 cells per well, at least 3,000 cells per well, at least 3,500 cells per well, at least 4,000 cells per well, at least 4,500 cells per well, at least 5,000 cells per well, at least 5,500 cells per well, at least 6,000 cells per well, at least 6,500 cells per well, at least 7,000 cells per well, or at least 5 cells per well.

In one embodiment, the organoids, for example, the bladder cell organoids, are prepared for passaging by releasing the organoids from the embedded Matrigel. In one embodiment, the Matrigel is dissolved by addition of Dispase to each well. In one embodiment, Dispase is added to the Matrigel matrix after removal of the overlaid liquid culture medium. In one embodiment, the Dispase is added at a final concentration of 1 mg/ml for 30 minutes at 37° C. In another embodiment, the final concentration of dispase is at least 0.2 mg/ml, at least 0.3 mg/ml, at least 0.4 mg/ml, at least 0.5 mg/ml, at least 0.6 mg/ml, at least 0.7 mg/ml, at least 0.8 mg/ml, at least 0.9 mg/ml, at least 1.0 mg/ml, at least 1.5 mg/ml, at least 2.0 mg/ml, at least 2.5 mg/ml, or at least 3 mg/ml. In one embodiment, the cells are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 22 minutes, at least 23 minutes, at least 24 minutes, at least 25 minutes, at least 26 minutes, at least 27 minutes, at least 28 minutes, at least 29 minutes, at least 30 minutes, at least 40 minutes, at least 50 minutes, or at least 60 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, the dispase solution is discarded and residual Matrigel is removed with cold PBS.

In one embodiment the Dispase activity is stopped by the addition of HBSS containing 2% FBS. In one embodiment, the HBSS does not contain Ca²⁺. In another embodiment, the HBSS does not contain Mg′. In one embodiment, the HBSS contains Ca²⁺. In another embodiment, the HBSS contains Mg′. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

The released organoids, for example, released bladder organoids, are separated from the Dispase containing medium by centrifugation. In one embodiment, the released organoids can be washed in 1× Phosphate Buffered Saline (PBS).

In one embodiment, the organoids, for example, the bladder cell organoids, are prepared for passaging by releasing the organoids from the embedded collagen. In one embodiment, the collagen is dissolved by addition of collagenase to each well. In one embodiment, collagenase is added to the collagen matrix after removal of the overlaid liquid culture medium. In one embodiment, the collagenase is added at a final concentration of 0.25 mg/ml for 30 minutes at 37° C. In another embodiment, the final concentration of dispase is at least 0.1 mg/ml, at least 0.3 mg/ml, at least 0.4 mg/ml, at least 0.5 mg/ml, at least 0.6 mg/ml, at least 0.7 mg/ml, at least 0.8 mg/ml, at least 0.9 mg/ml, or at least 1.0 mg/ml. In one embodiment, the cells are incubated for at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 16 minutes, at least 17 minutes, at least 18 minutes, at least 19 minutes, at least 20 minutes, at least 22 minutes, at least 23 minutes, at least 24 minutes, at least 25 minutes, at least 26 minutes, at least 27 minutes, at least 28 minutes, at least 29 minutes, at least 30 minutes, at least 40 minutes, at least 50 minutes, or at least 60 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, the collagenase solution is discarded and residual collagen is removed with cold PBS.

In one embodiment the collagenase activity is stopped by the addition of HBSS containing 2% FBS. In one embodiment, the HBSS does not contain Ca²⁺. In another embodiment, the HBSS does not contain Mg²⁺. In one embodiment, the HBSS contains Ca²⁺. In another embodiment, the HBSS contains Mg²⁺. In a further embodiment, the HBSS contains 10 mM HEPES. In one embodiment, the HBSS does not contain phenol red. In another embodiment, the HBSS does contain phenol red. In one embodiment, the HBSS contains at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

The released organoids, for example, released bladder organoids, are separated from the collagenase containing medium by centrifugation. In one embodiment, the released organoids can be washed in 1× Phosphate Buffered Saline (PBS).

In one embodiment, the released organoids, for example, the released bladder cell organoids, are dissociated into cell clusters by addition of TrypLE™. In one embodiment, the 1× TrypLE™ is added for 1 minute at 25° C. In one embodiment, the cells are incubated for at least 1 minute, at least 2 minutes, at least 3 minutes, at least 4 minutes, or at least 5 minutes. In one embodiment, the sample is incubated at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In one embodiment, the cell clusters are plated as described for the Matrigel embedding method.

In one embodiment, the dissociated cell clusters are frozen by resuspending the cell clusters in a freezing media. In one embodiment, the freezing media comprises hepatocyte medium, FBS, and DMSO. In one embodiment, the freezing media contains about 50% FBS, about 40% hepatocyte media, and about 10% DMSO. In one embodiment, the FBS is heat-inactivated charcoal-stripped FBS. In one embodiment, cells are gradually frozen to less than or equal to −80° C.

In one embodiment, frozen cells, for example, frozen bladder cell organoid clusters, can be thawed. In one embodiment, the frozen cells are thawed rapidly in at about 37° C. and immediately diluted in HBSS containing 2% FBS. In one embodiment, the thawed cells are immediately separated from the freezing media by centrifugation. In one embodiment, the cell clusters are plated as described for the Matrigel embedding method.

In one embodiment, organoids, for example, bladder organoids can be converted to two-dimensional adherent culture. In one embodiment, bladder organoids can be converted at any point after successful establishment of primary organoid cultures. In one embodiment, bladder organoids can be converted after passaging of organoids. In one embodiment, the released organoids, for example, the released bladder cell organoids, are dissociated into single cells and converted to two-dimensional adherent culture. For example, in one embodiment, after passaging, Accutase™ treated bladder organoids, are separated from the Accutase™ containing medium by centrifugation. In another embodiment, the dissociated bladder organoid cells are plated into wells of a Primaria™ 24 well flat bottom surface modified multiwell cell culture plate. In another embodiment, the dissociated bladder organoid cells are plated in wells of a plate that enhances or maximizes attachment of the cells to the wells. In another embodiment, the plate is a polystyrene plate. In a further embodiment, the plate is a surface modified polystyrene plate. Without being bound by theory, the surface of the plate can be modified to incorporate anionic and cationic functional groups to enhance the attachment of the cells to the surface if the plate.

In one embodiment, the cells are plated into a wells of a 24 well plate at a final density of 75,000 cells per well. In another embodiment, the cells are plated into wells of a 24 well plate at a final density of about 50,000 cells per well, about 55,000 cells per well, about 60,000 cells per well, about 65,000 cells per well, about 70,000 cells per well, about 75,000 cells per well, about 80,000 cells per well, about 85,000 cells per well, about 90,000 cells per well, about 95,000 cells per well, or about 100,000 cells per well. Without being bound by theory, a well of a 24 well plate has a surface area of about 1.9 cm².

In another embodiment, cells are plated into wells of a 24 well plate at a final density of at least 50,000 cells per well, at least 55,000 cells per well, at least 60,000 cells per well, at least 65,000 cells per well, at least 70,000 cells per well, at least 75,000 cells per well, at least 80,000 cells per well, at least 85,000 cells per well, at least 90,000 cells per well, at least 95,000 cells per well, or at least 100,000 cells per well.

In one embodiment, organoids, for example, bladder organoids can be frozen. In one embodiment, bladder organoids can be frozen at any point after successful establishment of primary organoid cultures. In one embodiment, bladder organoids can be frozen after passaging of organoids. In one embodiment, Accutase™ treated organoids, for example, Accutase™ treated bladder organoids, are separated from the Accutase™ containing medium by centrifugation. In one embodiment, the dissociated organoid cells are frozen by resuspending the detached cells in a freezing media. In one embodiment, the freezing media comprises hepatocyte medium, FBS, and DMSO. In one embodiment, the freezing media contains about 50% FBS, about 40% hepatocyte media, and about 10% DMSO. In one embodiment, the FBS is heat-inactivated charcoal-stripped FBS. In one embodiment, cells are gradually frozen to less than or equal to −80° C.

In one embodiment, frozen cells, for example, frozen bladder cell lines, can be thawed. In one embodiment, the frozen cells are thawed rapidly in at about 37° C. and immediately diluted in HBSS containing 2% FBS. In one embodiment, the thawed cells are immediately separated from the freezing media by centrifugation. In one embodiment, the cells are plated into a new 96 well low attachment plate.

In another embodiment, epithelial cells, for example, bladder organoids, can be cultured to generate organoids using a Matrigel™ embedding method. In one embodiment, epithelial cells are suspended in hepatocyte medium. In one embodiment, the hepatocyte culture medium is supplemented with 10 ng/ml of EGF. In one embodiment, the hepatocyte culture medium is supplemented with about 1 ng/ml of EGF, 2 ng/ml of EGF, 3 ng/ml of EGF, 4 ng/ml of EGF, 5 ng/ml of EGF, 6 ng/ml of EGF, 7 ng/ml of EGF, 8 ng/ml of EGF, 9 ng/ml of EGF, 10 ng/ml of EGF, 11 ng/ml of EGF, 12 ng/ml of EGF, 13 ng/ml of EGF, 14 ng/ml of EGF, 15 ng/ml of EGF, 16 ng/ml of EGF, 17 ng/ml of EGF, 18 ng/ml of EGF, 19 ng/ml of EGF, about 20 ng/ml of EGF, about 25 ng/ml of EGF, about 30 ng/ml of EGF, about 35 ng/ml of EGF, about 40 ng/ml of EGF, about 45 ng/ml of EGF, about 50 ng/ml of EGF, or more.

In another embodiment, the hepatocyte culture medium is supplemented with at least 1 ng/ml of EGF, at least 2 ng/ml of EGF, at least 3 ng/ml of EGF, at least 4 ng/ml of EGF, at least 5 ng/ml of EGF, at least 6 ng/ml of EGF, at least 7 ng/ml of EGF, at least 8 ng/ml of EGF, at least 9 ng/ml of EGF, at least 10 ng/ml of EGF, at least 15 ng/ml of EGF, at least 20 ng/ml of EGF, at least 30 ng/ml of EGF, at least 40 ng/ml of EGF, or at least 50 ng/ml of EGF.

In one embodiment, the hepatocyte culture medium is supplemented with 2 mM of GlutaMAX™. GlutaMAX™ is the dipeptide L-alanyl-L-glutamine. In one embodiment, the hepatocyte culture medium is supplemented with at least 0.1 mM of GlutaMAX™, at least 0.5 mM of GlutaMAX™, at least 1 mM of GlutaMAX™, at least 1.5 mM of GlutaMAX™, at least 2 mM of GlutaMAX™, at least 3 mM of GlutaMAX™, at least 4 mM of GlutaMAX™, or at least 5 mM of GlutaMAX™. In another embodiment, the hepatocyte culture medium is supplemented with L-glutamine.

In one embodiment, the hepatocyte culture medium is not supplemented with Matrigel™. In one embodiment, the hepatocyte culture medium is supplemented with Matrigel™.

In one embodiment, the hepatocyte culture medium is supplemented with 5% FBS. In another embodiment, the FBS is heat-inactivated charcoal-stripped FBS (e.g. Gibco, cat #12676). In one embodiment, the hepatocyte culture medium is supplemented with about 0.1% FBS, about 0.2% FBS, about 0.3% FBS, about 0.4% FBS, about 0.5% FBS, about 0.6% FBS, about 0.7% FBS, about 0.8% FBS, about 0.9% FBS, about 1% FBS, about 2% FBS, about 3% FBS, about 4% FBS, about 5% FBS, about 6% FBS, about 7% FBS, about 8% FBS, about 9% FBS, about 10% FBS, about 15% FBS, or about 20% FBS, or more.

In one embodiment, the hepatocyte culture medium is supplemented with at least 0.1% FBS, at least 0.2% FBS, at least 0.3% FBS, at least 0.4% FBS, at least 0.5% FBS, at least 0.6% FBS, at least 0.7% FBS, at least 0.8% FBS, at least 0.9% FBS, at least 1% FBS, at least 2% FBS, at least 3% FBS, at least 4% FBS, at least 5% FBS, at least 6% FBS, at least 7% FBS, at least 8% FBS, at least 9% FBS, at least 10% FBS, or at least 20% FBS.

In one embodiment, the hepatocyte culture medium is supplemented with a Rho-Associated Coil Kinase (ROCK) inhibitor. In one embodiment, the ROCK inhibitor is Y-27632. In one embodiment, the hepatocyte culture medium is supplemented with 1004 of Y-27632. In another embodiment, the hepatocyte culture medium is supplemented with about 1 μM of Y-27632, about 2 μM of Y-27632, about 3 μM of Y-27632, about 4 μM of Y-27632, about 5 μM of Y-27632, about 6 μM of Y-27632, about 7 μM of Y-27632, about 8 μM of Y-27632, about 9 μM of Y-27632, about 1004 of Y-27632, about 11 μM of Y-27632, about 1204 of Y-27632, about 1304 of Y-27632, about 1404 of Y-27632, about 15 μM of Y-27632, about 2004 of Y-27632, about 3004 of Y-27632, about 4004 of Y-27632, or about 5004 of Y-27632.

In another embodiment, the hepatocyte culture medium is supplemented with at least 1 μM of Y-27632, at least 2 μM of Y-27632, at least 3 μM of Y-27632, at least 4 μM of Y-27632, at least 5 μM of Y-27632, at least 6 μM of Y-27632, at least 7 μM of Y-27632, at least 8 μM of Y-27632, at least 9 μM of Y-27632, at least 1004 of Y-27632, at least 11 μM of Y-27632, at least 1204 of Y-27632, at least 1304 of Y-27632, at least 1404 of Y-27632, at least 15 μM of Y-27632, at least 20 μM of Y-27632, at least 30 μM of Y-27632, at least 40 μM of Y-27632, or at least 50 μM of Y-27632.

In one embodiment, the epithelial cells, for example, bladder epithelial cells, are suspended in Matrigel™. In one embodiment, the epithelial cell-Matrigel™ suspension is plated around the rim of tissue culture plates. In one embodiment, the tissue culture plate is a 24 well plate. In one embodiment, after the Matrigel™ solidifies, culture media is added to the wells.

In one embodiment, a change of media occurs every 4 days. In one embodiment, the change of media is a half-changed of media. In another embodiment, the change of media is a full change of media. In another embodiment, a change of media occurs at least every day, at least every 2 days, at least every 3 days, at least every 4 days, at least every 5 days, at least every 6 days, at least every 7 days, at least every 8 days, at least every 9 days, at least every 10 days, at least every 11 days, at least every 12 days, at least every 13 days, or at least every 14 days.

### Bladder Cell Lines

In one aspect, the invention provides a bladder cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture. In one embodiment, the subject is a human. In another embodiment, the cell line is preserved in a tissue bank.

In one aspect, the invention provides a bladder cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; (e) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids. In one embodiment, the subject is a human. In another embodiment, the cell line is preserved in a tissue bank.

In one aspect, the invention provides a bladder cell line, wherein the cell line is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids, and wherein a bladder cell line is obtained from the organoids. In one embodiment, the subject is a human. In another embodiment, the cell line is preserved in a tissue bank.

In one aspect, the invention provides a bladder cell line, wherein the cell line is obtained by the method comprising: (a) obtaining a sample of bladder tissue from a subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; and (e) incubating the culture of (d) wherein the dissociated bladder tissue forms organoids, and wherein a bladder cell line is obtained from the organoids. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on an adherent cell culture support; and (e) culturing the dissociated bladder epithelial cells in a culture medium comprising hepatocyte medium, FBS, Matrigel, and ROCK inhibitor; wherein the dissociated bladder epithelial cells form bladder cell line colonies in culture. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids, and wherein a bladder cell line is obtained from the organoids. In one embodiment, the bladder tumor cell line displays the transformed phenotype of cancerous bladder tissue. In one embodiment, the subject is a human. In another embodiment, the cell line is preserved in a tissue bank.

In one aspect, the invention provides a bladder tumor cell line, wherein the cell line is obtained by the method comprising: (a) obtaining a sample of bladder tissue from a subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; and (e) incubating the culture of (d) wherein the dissociated bladder tissue forms organoids, and wherein a bladder cell line is obtained from the organoids. In one embodiment, the bladder tumor cell line displays the transformed phenotype of cancerous bladder tissue. In one embodiment, the subject is a human. In another embodiment, the cell line is preserved in a tissue bank.

In one embodiment, epithelial cells, for example, bladder epithelial cells, can be cultured to generate bladder cell lines. In one embodiment, bladder cell lines can be grown for at least 3 weeks. In further embodiments, bladder organoids can be growth for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, or at least 8 months.

In one embodiment, the method can comprise analyzing the phenotype of bladder cell lines by detecting the presence of a marker gene (such as, but not limited to, CK5, CK8, CK7, UP3, Ki67, and p53) polypeptide expression. Polypeptide expression includes the presence of a marker gene polypeptide sequence, or the presence of an elevated quantity of marker gene polypeptide as compared to non-epithelial cells. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (such as, but not limited to, CK5, CK8, CK7, UP3, Ki67, and p53) RNA expression, in cell lines, for example in bladder cell lines. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

### Bladder Organoids

In one aspect, the invention provides a bladder organoid, wherein the organoid is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture. In one embodiment, the subject is a human. In another embodiment, the cell line is preserved in a tissue bank.

In one aspect, the invention provides a bladder organoid, wherein the organoid is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids. In one embodiment, the subject is a human. In another embodiment, the cell line is preserved in a tissue bank.

In one aspect, the invention provides a bladder organoid, wherein the organoid is obtained by the method comprising: (a) obtaining a sample of bladder tissue from a subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; and (e) incubating the culture of (d) wherein the dissociated bladder tissue forms organoids. In one embodiment, the subject is a human. In another embodiment, the cell line is preserved in a tissue bank.

In one aspect, the invention provides a bladder tumor organoid, wherein the organoid is obtained by the method comprising: (a) obtaining a sample of bladder tissue from a subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; and (e) culturing the dissociated bladder epithelial cells in a culture medium comprising hepatocyte medium, FBS, Matrigel, and ROCK inhibitor; wherein the dissociated bladder epithelial cells form organoids in culture.

In one aspect, the invention provides a bladder tumor organoid, wherein the organoid is obtained by the method comprising: (a) obtaining a sample of bladder tissue from a subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; and (e) incubating the culture of (d) wherein the dissociated bladder tissue forms organoids.

In one aspect, the invention provides a bladder tumor organoid, wherein the organoid is obtained by the method comprising: (a) obtaining a sample of bladder tissue from a subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; and (e) incubating the culture of (d) wherein the dissociated bladder tissue forms organoids.

In one embodiment, the bladder organoid displays the transformed phenotype of cancerous bladder tissue. In one embodiment, the subject is a human. In another embodiment, the cell line is preserved in a tissue bank.

In one embodiment, epithelial cells, for example, bladder epithelial cells, can be cultured to generate organoids using a Matrigel™ floating method. In another embodiment, bladder epithelial cells can be cultured to generate organoids using a Matrigel™ embedding method. In another embodiment, bladder epithelial cells can be cultured to generate organoids using a collagen embedding method. In one embodiment, bladder organoids can be grown for at least 3 weeks. In further embodiments, bladder organoids can be growth for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, or at least 8 months.

In one embodiment, the method can comprise analyzing the phenotype of organoids by detecting the presence of a marker gene (such as, but not limited to, CK5, CK8, CK7, UP3, Ki67, and p53) polypeptide expression. Polypeptide expression includes the presence of a marker gene polypeptide sequence, or the presence of an elevated quantity of marker gene polypeptide as compared to non-epithelial cells. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (such as, but not limited to, CK5, CK8, CK7, UP3, Ki67, and p53) RNA expression, in organoids, for example in bladder organoids. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

### Methods of Screening Compounds

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on an adherent cell culture support; and (v) culturing the dissociated bladder epithelial cells in a culture medium comprising hepatocyte medium, FBS, Matrigel, and ROCK inhibitor; wherein the dissociated bladder epithelial cells form bladder cell line colonies in culture; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids, and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder cell line with a test compound, wherein the cell line is obtained by the method comprising: (i) obtaining a sample of bladder tissue from a subject; (ii) dissociating the sample of bladder tissue; (iii) contacting the dissociated bladder tissue with a collagen solution and plating in a cell culture support, wherein the collagen solution forms a matrix; (iv) providing an overlay layer of liquid culture medium comprising hepatocyte medium and FBS; and (v) incubating the culture of (iv) wherein the dissociated bladder tissue forms organoids, and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on an adherent cell culture support; and (v) culturing the dissociated bladder epithelial cells in a culture medium comprising hepatocyte medium, FBS, Matrigel, and ROCK inhibitor; wherein the dissociated bladder epithelial cells form bladder cell line colonies in culture; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids, and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor cell line with a test compound, wherein the cell line is obtained by the method comprising: (i) obtaining a sample of bladder tissue from a subject; (ii) dissociating the sample of bladder tissue; (iii) contacting the dissociated bladder tissue with a collagen solution and plating in a cell culture support, wherein the collagen solution forms a matrix; (iv) providing an overlay layer of liquid culture medium comprising hepatocyte medium and FBS; and (v) incubating the culture of (iv) wherein the dissociated bladder tissue forms organoids, and wherein a bladder cell line is obtained from the organoids; and (b) determining whether growth of the cell line is inhibited in the presence of the test compound, as compared to growth of the cell line in the absence of the test compound; wherein inhibition of growth of the cell line indicates the identification of a compound that inhibits bladder cancer. In one embodiment, the test compound is a small molecule.

In one aspect, the invention provides a method for identifying a compound that inhibits bladder cancer, the method comprising: (a) contacting a bladder organoid with a test compound, wherein the organoid is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder organoid with a test compound, wherein the organoid is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder organoid with a test compound, wherein the organoid is obtained by the method comprising: (i) obtaining a sample of bladder tissue from a subject; (ii) dissociating the sample of bladder tissue; (iii) contacting the dissociated bladder tissue with a collagen solution and plating in a cell culture support, wherein the collagen solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor organoid with a test compound, wherein the organoid is obtained by the method comprising: (i) obtaining a sample of bladder tissue from a subject; (ii) dissociating the sample of bladder tissue; (iii) isolating dissociated bladder epithelial cells from the sample of bladder tissue; (iv) plating the isolated dissociated bladder epithelial cells of (iii) on a low attachment cell culture support; and (v) culturing the dissociated bladder epithelial cells in a culture medium comprising hepatocyte medium, FBS, Matrigel, and ROCK inhibitor; wherein the dissociated bladder epithelial cells form organoids in culture; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor organoid with a test compound, wherein the organoid is obtained by the method comprising: (i) obtaining a sample of bladder tissue from a subject; (ii) dissociating the sample of bladder tissue; (iii) contacting the dissociated bladder tissue with a Matrigel solution and plating in a cell culture support, wherein the Matrigel solution comprises hepatocyte medium and Matrigel and wherein the Matrigel solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one aspect, the invention provides a method for identifying a compound that inhibits bladder cancer, the method comprising: (a) contacting a bladder tumor organoid with a test compound, wherein the organoid is obtained by the method comprising: (i) obtaining a sample of bladder tissue from a subject; (ii) dissociating the sample of bladder tissue; (iii) contacting the dissociated bladder tissue with a collagen solution and plating in a cell culture support, wherein the collagen solution forms a matrix; (iv) providing an overlay layer of liquid culture medium comprising hepatocyte medium and FBS; and (v) incubating the culture of (iv) wherein the dissociated bladder tissue forms organoids; and (b) determining whether growth of the organoid is inhibited in the presence of the test compound, as compared to growth of the organoid in the absence of the test compound; wherein inhibition of growth of the organoid indicates the identification of a compound that inhibits bladder cancer. In one embodiment, the test compound is a small molecule.

In one embodiment, the test compound is an intravesical agent. In another embodiment, the test compound is an antineoplastic agent. In a further embodiment, the test compound is a chemotherapy agent. In one embodiment, the test compound is Docetaxel. In one embodiment, the test compound is Gemcitabine. In another embodiment, the test compound is Mitomycin. In another embodiment, the test compound is Rapamycin.

In one embodiment, the test compound is a small molecule. In another embodiment, the test compound is a peptide. In one embodiment, the test compound is a protein. In another embodiment, the test compound is a peptidomimetic molecule. In yet another embodiment, the test compound is an antibody.

The invention provides for methods used to identify compounds that inhibit cancer. The method can further comprise determining whether the growth of bladder cancer cell lines organoids is inhibited in the presence of a test compound as compared to growth of the bladder cancer cell lines or organoids in the absence of the test compound.

Test compounds can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries, neurotransmitter libraries, and chemical libraries. The libraries can also comprise cyclic carbon or heterocyclic structure and/or aromatic or polyaromatic structures substituted with one or more of the functional groups.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

### Methods of Treatment

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; (d) plating the isolated dissociated bladder epithelial cells of (c) on an adherent cell culture support; (e) culturing the dissociated bladder epithelial cells in a culture medium comprising hepatocyte medium, FBS, Matrigel, and ROCK inhibitor, wherein the dissociated bladder epithelial cells form bladder cell line colonies in culture; (f) contacting the bladder cell line with a test compound; and (g) determining whether growth of the bladder cell line is inhibited in the presence of the test compound, as compared to growth of the bladder cell line in the absence of the test compound, wherein the test compound is administered to the subject if growth of the bladder cell line is inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; (d) plating the isolated dissociated bladder epithelial cells of (c) on an adherent cell culture support; (e) culturing the dissociated bladder epithelial cells in a culture medium comprising hepatocyte medium, FBS, Matrigel, and ROCK inhibitor, wherein the dissociated bladder epithelial cells form bladder cell line colonies in culture; (f) contacting the bladder cell line with a test compound; and (g) determining whether growth of the bladder cell line is inhibited in the presence of the test compound, as compared to growth of the bladder cell line in the absence of the test compound, wherein a cystectomy is performed on the subject if growth of the bladder cell line is not inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; (e) culturing the dissociated bladder epithelial cells in a culture medium comprising hepatocyte medium, FBS, Matrigel, and ROCK inhibitor, wherein the dissociated bladder epithelial cells form bladder organoids in culture; (f) contacting the bladder organoid with a test compound; and (g) determining whether growth of the bladder organoid is inhibited in the presence of the test compound, as compared to growth of the bladder organoid in the absence of the test compound, wherein the test compound is administered to the subject if growth of the bladder organoid is inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) isolating dissociated bladder epithelial cells from the sample of bladder tissue; (d) plating the isolated dissociated bladder epithelial cells of (c) on a low attachment cell culture support; (e) culturing the dissociated bladder epithelial cells in a culture medium comprising hepatocyte medium, FBS, Matrigel, and ROCK inhibitor, wherein the dissociated bladder epithelial cells form bladder organoids in culture; (f) contacting the bladder organoid with a test compound; and (g) determining whether growth of the bladder organoid is inhibited in the presence of the test compound, as compared to growth of the bladder organoid in the absence of the test compound, wherein a cystectomy is performed on the subject if growth of the bladder organoid is not inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; (e) incubating the culture of (d) wherein the dissociated bladder tissue forms organoids; (f) contacting the bladder organoid with a test compound; and (g) determining whether growth of the bladder organoid is inhibited in the presence of the test compound, as compared to growth of the bladder organoid in the absence of the test compound, wherein the test compound is administered to the subject if growth of the bladder organoid is inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a Matrigel solution and plating in a cell culture support, wherein the Matrigel solution comprises hepatocyte medium and Matrigel and wherein the Matrigel solution forms a matrix; (d) providing an overlay layer of liquid culture medium comprising hepatocyte medium and FBS; (e) culturing the dissociated bladder epithelial cells in a culture medium comprising hepatocyte medium, FBS, Matrigel, and ROCK inhibitor, wherein the dissociated bladder epithelial cells form bladder organoids in culture; (f) contacting the bladder organoid with a test compound; and (g) determining whether growth of the bladder organoid is inhibited in the presence of the test compound, as compared to growth of the bladder organoid in the absence of the test compound, wherein a cystectomy is performed on the subject if growth of the bladder organoid is not inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; (e) incubating the culture of (d) wherein the dissociated bladder tissue forms organoids; (f) contacting the bladder organoid with a test compound; and (g) determining whether growth of the bladder organoid is inhibited in the presence of the test compound, as compared to growth of the bladder organoid in the absence of the test compound, wherein the test compound is administered to the subject if growth of the bladder organoid is inhibited in the presence of the test compound.

In one aspect, the invention provides a method for treating bladder cancer in a subject in need thereof, comprising: (a) obtaining a sample of bladder tissue from the subject; (b) dissociating the sample of bladder tissue; (c) contacting the dissociated bladder tissue with a collagen solution and plating in a cell culture support, wherein the collagen solution forms a matrix; (d) providing an overlay layer of liquid culture medium comprising hepatocyte medium and FBS; (e) culturing the dissociated bladder epithelial cells in a culture medium comprising hepatocyte medium, FBS, Matrigel, and ROCK inhibitor, wherein the dissociated bladder epithelial cells form bladder organoids in culture; (f) contacting the bladder organoid with a test compound; and (g) determining whether growth of the bladder organoid is inhibited in the presence of the test compound, as compared to growth of the bladder organoid in the absence of the test compound, wherein a cystectomy is performed on the subject if growth of the bladder organoid is not inhibited in the presence of the test compound.

In one embodiment, the test compound is an intravesical agent. In another embodiment, the test compound is an antineoplastic agent. In a further embodiment, the test compound is a chemotherapy agent. In one embodiment, the test compound is Docetaxel. In one embodiment, the test compound is Gemcitabine. In another embodiment, the test compound is Mitomycin. In another embodiment, the test compound is Rapamycin. In another embodiment, the growth of the bladder cell line of (f) is measured using a MTT assay.

The dose(s) of a test compound to be administered according to the methods described herein can vary, for example, not only depending upon the growth of bladder cell lines or organoids.

The standard dose (s) of a test compound to be administered according to the methods described herein can vary, for example, depending upon the identity, size, and condition of the subject being treated and can further depend upon the route by which a test compound according to the methods described herein, is to be administered, if applicable, and the effect which the practitioner desires the a test compound according to the invention to have upon the target of interest. These amounts can be readily determined by one of skill in the art. Any of the therapeutic applications described herein can be applied to any subject in need of such therapy, including, for example, a mammal such as a human.

Appropriate dosing regimens can also be determined by one of skill in the art without undue experimentation, in order to determine, for example, whether to administer the agent in one single dose or in multiple doses, and in the case of multiple doses, to determine an effective interval between doses.

In certain embodiments, a test compound to be administered according to the methods described herein can be administered alone, or in combination with other drugs therapies, small molecules, biologically active or inert compounds, or other additive intended to enhance the delivery, efficacy, tolerability, or function of the test compound.

Therapy dose and duration will depend on a variety of factors, such as the disease type, patient age, therapeutic index of the drugs, patient weight, and tolerance of toxicity. The skilled clinician using standard pharmacological approaches can determine the dose of a particular therapeutic and duration of therapy for a particular patient in view of the above stated factors. The response to treatment can be monitored by one of skill in the art, such as a clinician, who can adjust the dose and duration of therapy based on the response to treatment revealed by these measurements.

In one embodiment, the bladder cancer is a transitional cell carcinoma or a urothelial cell carcinoma. In another embodiment, the bladder cancer is a squamous cell carcinoma. In another embodiment, the bladder cancer is adenocarcinoma. In one embodiment, the epithelium of the bladder is a transitional epithelium or urothelium.

Methods of Administering

Indications, dosage and methods of administration of the drugs of the present invention are known to one of skill in the art. In some embodiments, a drug of the present invention can be supplied in the form of a pharmaceutical composition, comprising an isotonic excipient prepared under sufficiently sterile conditions for human administration. Choice of the excipient and any accompanying elements of the composition will be adapted in accordance with the route and device used for administration. In some embodiments, a composition comprising a drug of the present invention can also comprise, or be accompanied with, one or more other ingredients that facilitate the delivery or functional mobilization of the drugs of the present invention.

These methods described herein are by no means all-inclusive, and further methods to suit the specific application is understood by the ordinary skilled artisan. Moreover, the effective amount of the compositions can be further approximated through analogy to compounds known to exert the desired effect.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} ed., 2000), the entire disclosure of which is herein incorporated by reference.

Any of the therapeutic applications described herein can be applied to any subject in need of such therapy, including, for example, a mammal such as a dog, a cat, a cow, a horse, a rabbit, a monkey, a pig, a sheep, a goat, or a human.

Administration of a drug of the present invention is not restricted to a single route, but may encompass administration by multiple routes. Multiple administrations may be sequential or concurrent. Other modes of application by multiple routes will be apparent to one of skill in the art.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Materials and Methods for Establishing Adherent Bladder Cell Cultures from Human Bladder Tissue

1.0 Introduction and Overview:

The protocol described herein is a new method for successfully establishing adherent culture from freshly-obtained human bladder tumor samples removed during routine endoscopic resection. The resected tumor tissue is dissociated into a single-cell suspension containing both epithelial and stromal cells. Epithelial cells are isolated from the parental population via immunomagnetic cell separation using antibodies against epithelial cell adhesion molecule (EpCAM, also CD326). The sorted epithelial cells are then seeded into 24-well plates in supplemented hepatocyte medium with 5% Matrigel. Once colonies have formed, these cultures can be serially passaged as well as frozen and thawed with resumed pre-freezing growth after thawing.

2.0 Materials

2.1 Specimen Preparation and Collagenase Digestion:

Freshly resected human bladder tumor tissue (0.1-2.0 grams of tissue, preferably removed without cautery)

Sterile 1×PBS (Gibco)

Gentamicin 50 mg/mL solution (Gibco)

10× Collagenase/hyaluronidase solution (Stemcell Technologies)

Dulbecco's Modified Eagle Medium: Nutrient Mixture F-12 (DMEM/F-12, Gibco), supplemented with 5% fetal bovine serum (FBS)

2.2 Enzymatic Dissociation to Single Cell Suspension:

Accutase Cell Detachment Solution (Stemcell Technologies)

Hanks' Balanced Salt Solution Modified (HBSS, Stemcell Technologies), supplemented with 2% FBS

Dispase 5 mg/mL (Stemcell Technologies)

DNaseI 1 mg/mL (Stemcell Technologies)

40 μm cell strainer (BD)

Hemacytometer with trypan blue (Gibco)

HBSS+2% FBS+10 μM ROCK inhibitor Y-27632 (Stemcell Technologies)

2.3 Immunomagnetic Cell Separation:

EasySep™ Human EpCAM Positive Selection Kit (Stemcell Technologies)

HBSS+2% FBS+10 μM ROCK Inhibitor Y-27632

DNaseI 1 mg/mL

2.4 Medium Preparation and Cell Plating:

Primaria™ 24 well flat bottom surface modified multiwall cell culture plate (Corning)

Hepatocyte culture media kit (with 10 ng/mL epidermal growth factor, Corning)

Heat-inactivated, charcoal stripped FBS (Invitrogen, see note 1)

100× Glutamax (Invitrogen)

Thawed Matrigel (Corning, see note 2)

5 mM ROCK inhibitor Y-27632

100× antibiotic-antimycotic (Gibco, optional, see note 3)

2.5 Passaging and Freezing Cells:

Cold phosphate buffered saline (PBS)

Accutase Cell Detachment Solution HBSS+2% FBS

Prepared media (see 2.4)

Heat-inactivated, charcoal stripped FBS

Dimethyl sulfoxide (DMSO, Sigma)

3.0 Procedure

3.1 Specimen Preparation and Collagenase Digestion:

3.1.1. Collect bladder tumor specimens endoscopically using either cold cup biopsy or loop cautery. Transfer resected tumor tissue into a 50 mL Falcon tube prefilled with 20 mL DMEM/F12+5% FBS (preferably in operative suite immediately after tissue is obtained). Keep tube on ice during transport to laboratory.

3.1.2. In tissue culture hood, combine 1 mL 10× collagenase/hyaluronidase mixture with 9 mL DMEM/F12+5% FBS. Place in 37° C. water bath until ready to use (Step 3.1.5. below).

3.1.3. Centrifuge specimen at 350 rcf for 2 minutes and discard supernatant. Wash twice with 10 mL cold PBS, centrifuging between washes.

3.1.4. Resuspend sample in 10 mL cold PBS supplemented with 5 mg/mL gentamicin (1 mL of 50 mg/mL gentamicin+9 mL PBS). Place on wrack on orbital shaker for 10 minutes at room temperature, then centrifuge at 350 rcf for 2 minutes and discard supernatant.

3.1.5. Resuspend in 10 mL of diluted pre-warmed collagenase/hyaluronidase solution.

3.1.6. Incubate in 37° C. incubator for 3 hours (see note 4).

3.2 Enzymatic Dissociation to Single Cell Suspension:

3.2.1. Centrifuge digested tissue at 350 rcf for 5 minutes and discard supernatant.

3.2.2. Resuspend pellet in 5 mL pre-warmed Accutase Cell Detachment Solution and incubate at 37° C. for 30 minutes.

3.2.3. During Accutase digestion, prepare dispase/DNaseI solution by adding 2004 DNaseI to 1.8 mL dispase. Place in 37° C. water bath until ready to use.

3.2.4. After Accutase digestion is complete (30 minutes), add 10 mL cold HBSS+2% FBS to quench reaction. Centrifuge at 350 rcf for 5 minutes and discard supernatant.

3.2.5. Add 2 mL of pre-warmed dispase/DNaseI solution. Pipette the sample vigorously for 1-2 minutes using P1000 pipette until solution is homogenously translucent with no visible tissue fragments. (Do not allow digestion to continue for more than 2 minutes.)

3.2.6. Add, 10 mL cold HBSS+2% FBS to quench reaction.

3.2.7. Filter cell suspension through a 40 μm cell strainer into a new 50 mL conical tube.

3.2.8. Centrifuge filtered suspension at 350 rcf for 5 minutes and discard supernatant.

3.2.9. Resuspend pellet in 1 mL HBSS+2% FBS+10 μM ROCK inhibitor Y-27632 and transfer to 1.5 mL Eppendorf tube.

3.2.10. Count viable cells using a hemacytometer and Trypan Blue (use 104 cell suspension, 404 HBSS+2% FBS, and 50 μL Trypan Blue; use 104 of solution for counting and account for 10× dilution in final quantification.).

3.2.11. Centrifuge and resuspend cells in HBSS+2% FBS+10 μM ROCK inhibitor Y-27632+0.1 mg/mL DNase I at 1×10⁸ cells/mL. (If fewer than 1×10⁷ cells are obtained, resuspend in 1004. Immunomagnetic selection protocol is designed for up to 2×10⁸ cells.)

3.3 Immunomagnetic Cell Separation:

*Keep cell suspension and reagents on ice until sorting is finished.

3.3.1. Perform incubations and immunomagnetic cell selection per protocol for the EasySep™ Human EpCAM Positive Selection Kit. (Use HBSS+2% FBS+10 μM ROCK inhibitor Y-27632 as “recommended medium” listed in protocol.)

3.3.2. After final separation, resuspend in 2 mL HBSS+2% FBS+10 μM ROCK inhibitor Y-27632. Count viable cells using a hemacytometer and Trypan Blue

3.4. Medium Preparation and Cell Plating

3.4.1. Prepare desired amount of culture medium by combining the following components (a-d can be combined and stored as a 50 mL aliquot in 4° C. refrigerator for up to 4 weeks; e-g should be added on the day of use based on the amount of media needed):

a. Hepatocyte Medium (47 mL per 50 mL media)
b. 10 ng/mL EGF (1004 of 5 μg/mL stock per 50 mL media)
c. 5% Heat-inactivated, charcoal-stripped FBS (2.5 mL per 50 mL media)
d. 100× Glutamax (5004 per 50 mL media)
e. 5% Matrigel (504 per 1 mL media)
f 10 μM ROCK inhibitor Y-27632 (24 of 5 mM stock per 1 mL media)
g. 100× Antibiotic-antimycotic (10 uL per 1 mL media), optional (see note 3)

3.4.2. Keep prepared culture media at room temperature until use (rapid warming in 37° C. water bath may cause Matrigel to solidify at top of tube).

3.4.3. Centrifuge sorted cells at 350 rcf for 5 minutes and resuspend in prepared media at 75,000 cells per 5004 media.

3.4.4. Add resuspended cells to Primaria™ 24 well flat bottom surface modified multiwall cell culture plate at 5004 per well for a final plating density of 75,000 cells per well.

3.4.5. Change media every 4 days by removing all old media and adding 5004 fresh media to each well. When cells have reached 75% confluence or after 12 days (whichever occurs first), passage cells (see below).

3.5 Passaging and Freezing Cells:

3.5.1. To passage cells, begin by adding pre-warmed dispase to each well for a final dispase concentration of 1 mg/mL (typically approximately 3004 of 5 mg/mL dispase solution is appropriate.). Incubate in 37° C. incubator for 10 minutes. Discard supernatant.

3.5.2. Wash wells in cold PBS to finish removing Matrigel layer. If residual Matrigel remains on the bottom surface of plate, spray cold PBS onto the surface with a P1000 pipette tip; remove and discard any remaining supernatant.

3.5.3. Add 1 mL warm Accutase Cell Detachment Solution and incubate in 37° C. incubator for 15 minutes.

3.5.4. Pipette and spray bottom of each well several times with the Accutase in the corresponding well using P1000 pipet tip to loosen remaining attached cells.

3.5.5. Transfer pooled detached cell suspension into a 50 mL conical tube prefilled with an equal amount of cold HBSS+2% FBS.

3.5.6. Centrifuge at 350 rcf for 5 minutes and discard supernatant.

3.5.7. Resuspend cell pellet in fresh media and plate into a new Primaria™ 24 well flat bottom surface modified multiwall cell culture plate. In general, cells can be split at a 3 or 4:1 surface area ratio of the previous passage. Passage from 24 to 6 well plates when necessary.

3.5.8. Cells can be frozen at any point during a passage cycle. Steps 1-6 are identical, but the final cell pellet is resuspended in freezing media (50% heat-inactivated charcoal-stripped FBS, 40% hepatocyte media, 10% DMSO), typically 1 mL per 2 wells on a 6 well plate, or 1 mL per 8 wells on a 24 well plate. Transfer cells in 1 mL aliquots 1.8 mL cryo tubes. Gradual even freezing to ≦−80° using an insulated cryo freezing container is recommended. Cells should be thawed rapidly in a 37° C. water bath and immediately diluted in 10 mL HBSS+2% FBS per 1 mL freezing media. Spin thawed cells at 350 rcf for 5 minutes and resuspend in the appropriate amount of fresh culture media for plating.

4.0 Notes:

Note 1: Charcoal-stripped FBS must be heat-inactivated prior to use. Heat in 55° C. water bath for 60 min. Heat-inactivated charcoal-stripped FBS can be aliquotted and stored at −20° C.

Note 2: Matrigel must remain ≦4° C. at all times until use to prevent polymerization. It is recommend to place the Matrigel in 4° C. refrigerator overnight to thaw and keeping it on ice until it is added to media. Unused Matrigel can be refrozen, but avoid multiple freeze-thaw cycles.

Note 3: It is recommend to culture without antibiotics, but antibiotics can be added during initial culturing period or if there is increased concern for contamination from other sources.

Note 4: Shaking the tube periodically to redistribute bladder tissue is helpful.

### Example 2—Materials and Methods for Establishing Bladder Organoid

Cultures from Human Bladder Tissue

1.0 Introduction and Overview:

The protocol described herein is a new method for successfully establishing organoid culture from freshly-obtained human bladder tumor samples removed during routine endoscopic resection. The resected tumor tissue is dissociated into a single-cell suspension containing both epithelial and stromal cells. Epithelial cells are isolated from the parental population via immunomagnetic cell separation using antibodies against epithelial cell adhesion molecule (EpCAM, also CD326). The sorted epithelial cells are then seeded into 96-well low-attachment plates in supplemented hepatocyte medium with 5% Matrigel. Once organoids have formed, these cultures can be serially passaged as well as frozen and thawed with resumed pre-freezing growth after thawing.

2.0 Materials

2.1 Specimen Preparation and Collagenase Digestion:

Freshly resected human bladder tumor tissue (0.1-2.0 grams of tissue, preferably removed without cautery)

Sterile 1×PBS (Gibco)

Gentamicin 50 mg/mL solution (Gibco)

10× Collagenase/hyaluronidase solution (Stemcell Technologies)

Dulbecco's Modified Eagle Medium: Nutrient Mixture F-12 (DMEM/F-12, Gibco), supplemented with 5% fetal bovine serum (FBS)

2.2 Enzymatic Dissociation to Single Cell Suspension:

Accutase Cell Detachment Solution (Stemcell Technologies)

Hanks' Balanced Salt Solution Modified (HBSS, Stemcell Technologies), supplemented with 2% FBS

Dispase 5 mg/mL (Stemcell Technologies)

DNaseI 1 mg/mL (Stemcell Technologies)

40 μm cell strainer (BD)

Hemacytometer with trypan blue (Gibco)

HBSS+2% FBS+10 μM ROCK inhibitor Y-27632 (Stemcell Technologies)

2.3 Immunomagnetic Cell Separation:

EasySep™ Human EpCAM Positive Selection Kit (Stemcell Technologies)

HBSS+2% FBS+10 μM ROCK Inhibitor Y-27632

DNaseI 1 mg/mL

2.4 Medium Preparation and Cell Plating:

96-well low-attachment plate (Corning)

Hepatocyte culture media kit (with 10 ng/mL epidermal growth factor, Corning)

Heat-inactivated, charcoal stripped FBS (Invitrogen, see note 1)

100× Glutamax (Invitrogen)

Thawed Matrigel (Corning, see note 2)

5 mM ROCK inhibitor Y-27632

100× antibiotic-antimycotic (Gibco, optional, see note 3)

2.5 Passaging and Freezing Organoids:

Cold phosphate buffered saline (PBS)

Accutase Cell Detachment Solution HBSS+2% FBS

Prepared media (see 2.4)

Heat-inactivated, charcoal stripped FBS

Dimethyl sulfoxide (DMSO, Sigma)

2.6 Converting Organoid Culture to Two-Dimensional Adherent Culture

Cold phosphate buffered saline (PBS)

Accutase Cell Detachment Solution

HBSS+2% FBS

Prepared media (see 2.4)

Primaria™ 24-well flat bottom surface modified multiwall cell culture plate (Corning)

3.0 Procedure

3.1 Specimen Preparation and Collagenase Digestion:

3.1.1. Collect bladder tumor specimens endoscopically using either cold cup biopsy or loop cautery. Transfer resected tumor tissue into a 50 mL Falcon tube prefilled with 20 mL DMEM/F12+5% FBS (preferably in operative suite immediately after tissue is obtained). Keep tube on ice during transport to laboratory.

3.1.2. In tissue culture hood, combine 1 mL 10× collagenase/hyaluronidase mixture with 9 mL DMEM/F12+5% FBS. Place in 37° C. water bath until ready to use (Step 3.1.5. below).

3.1.3. Centrifuge specimen at 350 rcf for 2 minutes and discard supernatant.

Wash twice with 10 mL cold PBS, centrifuging between washes.

3.1.4. Resuspend sample in 10 mL cold PBS supplemented with 5 mg/mL gentamicin (1 mL of 50 mg/mL gentamicin+9 mL PBS). Place on rack on orbital shaker for 10 minutes at room temperature, then centrifuge at 350 rcf for 2 minutes and discard supernatant.

3.1.5. Resuspend in 10 mL of diluted pre-warmed collagenase/hyaluronidase solution.

3.1.6. Incubate in 37° C. incubator for 3 hours (see note 4).

3.2 Enzymatic dissociation to single cell suspension:

3.2.1. Centrifuge digested tissue at 350 rcf for 5 minutes and discard supernatant.

3.2.2. Resuspend pellet in 5 mL pre-warmed Accutase Cell Detachment Solution and incubate at 37° C. for 30 minutes.

3.2.3. During Accutase digestion, prepare dispase/DNaseI solution by adding 2004 DNaseI to 1.8 mL dispase. Place in 37° C. water bath until ready to use.

3.2.4. After Accutase digestion is complete (30 minutes), add 10 mL cold HBSS+2% FBS to quench reaction. Centrifuge at 350 rcf for 5 minutes and discard supernatant.

3.2.5. Add 2 mL of pre-warmed dispase/DNaseI solution. Pipette the sample vigorously for 1-2 minutes using P1000 pipette until solution is homogenously translucent with no visible tissue fragments. (Do not allow digestion to continue for more than 2 minutes.)

3.2.6. Add, 10 mL cold HBSS+2% FBS to quench reaction.

3.2.7. Filter cell suspension through a 40 μm cell strainer into a new 50 mL conical tube.

3.2.8. Centrifuge filtered suspension at 350 rcf for 5 minutes and discard supernatant.

3.2.9. Resuspend pellet in 1 mL HBSS+2% FBS+10 μM ROCK inhibitor Y-27632 and transfer to 1.5 mL Eppendorf tube.

3.2.10. Count viable cells using a hemacytometer and Trypan Blue (use 104 cell suspension, 404 HBSS+2% FBS, and 50 μL Trypan Blue; use 104 of solution for counting and account for 10× dilution in final quantification.).

3.2.11. Centrifuge and resuspend cells in HBSS+2% FBS+10 μM ROCK inhibitor Y-27632+0.1 mg/mL DNase I at 1×10⁸ cells/mL. (If fewer than 1×10⁷ cells are obtained, resuspend in 1004. Immunomagnetic selection protocol is designed for up to 2×10⁸ cells.)

3.3 Immunomagnetic Cell Separation:

*Keep cell suspension and reagents on ice until sorting is finished.

3.3.1. Perform incubations and immunomagnetic cell selection per protocol for the EasySep™ Human EpCAM Positive Selection Kit. (Use HBSS+2% FBS+10 μM ROCK inhibitor Y-27632 as “recommended medium” listed in protocol.)

3.3.2. After final separation, resuspend in 2 mL HBSS+2% FBS+10 μM ROCK inhibitor Y-27632. Count viable cells using a hemacytometer and Trypan Blue

3.4. Medium Preparation and Cell Plating

3.4.1. Prepare desired amount of culture medium by combining the following components (a-d can be combined and stored as a 50 mL aliquot in 4° C. refrigerator for up to 4 weeks; e-g should be added on the day of use based on the amount of media needed):

a. Hepatocyte Medium (47 mL per 50 mL media)
b. 10 ng/mL EGF (1004 of 5 μg/mL stock per 50 mL media)
c. 5% Heat-inactivated, charcoal-stripped FBS (2.5 mL per 50 mL media)
d. 100× Glutamax (5004 per 50 mL media)
e. 5% Matrigel (504 per 1 mL media)
f 10 μM ROCK inhibitor Y-27632 (2 μL of 5 mM stock per 1 mL media)
g. 100× Antibiotic-antimycotic (10 uL per 1 mL media), optional (see note 3)

3.4.2. Keep prepared culture media at room temperature until use (rapid warming in 37° C. water bath may cause Matrigel to solidify at top of tube).

3.4.3. Centrifuge sorted cells at 350 rcf for 5 minutes and resuspend in prepared media at 5,000 cells per 1004 media.

3.4.4. Add resuspended cells to 96-well low attachment plate at 1004 per well for a final plating density of 5,000 cells per well.

3.4.5. Change media every 4 days by adding 1004 fresh media to each well on days 4 and 8 after plating. On day 12 when wells are full (3004), transfer each well to a 1.5 ml Eppendorf tube and centrifuge at 250 rcf for 5 minutes. Remove 2004 of supernatant and add 1004 fresh media (total volume will be 2004). Transfer onto a new 96-well plate using P1000 pipet tip (smaller tips may damage organoids). Alternate every 4 days between either adding 1004 or spinning down to remove 2004 and add 1004 until ready to passage. (Multiple wells can be pooled prior to centrifuging and redistributed evenly if there are many wells.)

3.5 Passaging and Freezing Organoids:

3.5.1. When organoids are very large and media color pales rapidly after changing (usually 3-5 weeks after plating), prepare organoids for passage by transferring into 1.5 mL Eppendorf tubes and spinning at 250 rcf for 5 minutes. (Multiple wells can be pooled.) Discard supernatant.

3.5.2. Wash cells in cold PBS and spin again at 250 rcf for 5 minutes.

3.5.3. Add 1 mL warm Accutase Cell Detachment Solution and incubate in 37° C. water bath for 15 minutes.

3.5.4. Pipette up and down with P200 pipet tip for 30 seconds to dissociate cells.

3.5.5. Transfer suspension into a 15 mL conical tube prefilled with 2 mL cold HBSS+2% FBS.

3.5.6. Centrifuge at 350 rcf for 5 minutes and discard supernatant.

3.5.7. Resuspend cell pellet in fresh media and plate into a new low-attachment 96-well plate. Cells can be plated by either replating 4× the number of wells passaged in 1004 per well or by counting viable cells and replating at 5,000 cells/1004 media per well.

3.5.8. Organoids can be frozen at any point during a passage cycle by centrifuging at 250 rcf for 5 minutes and resuspending in 1 mL freezing media in 1.8 mL cryo tubes (50% heat-inactivated charcoal-stripped FBS, 40% hepatocyte media, 10% DMSO). Gradual even freezing to ≦−80° using an insulated cryo freezing container is recommended. Organoids should be thawed rapidly in a 37° C. water bath and immediately diluted in 10 mL HBSS+2% FBS per 1 mL freezing media. Spin thawed organoids at 250 rcf for 5 minutes and resuspend in organoid culture media for plating.

3.6 Converting Organoid Culture to Two-Dimensional Adherent Culture

3.6.1. Organoid culture can be converted to two-dimensional adherent culture at any point after successful establishment of primary culture. Begin by completing the first six steps of passaging (up through the centrifugation step) (3.5.1-3.5.6.).

3.6.2. Resuspend cell pellet in 1 mL HBSS+2% FBS. Count viable cells using a hemacytometer and Trypan Blue.

3.6.3. Centrifuge cells at 350 rcf for 5 minutes and resuspend in prepared media at 75,000 cells per 5004 media (see note 5).

3.6.4. Add resuspended cells to Primaria™ 24-well flat bottom surface modified multiwell cell culture plate at 5004 per well for a final plating density of 75,000 cells per well.

3.6.5. Continue to change media every 4 days and passage as per adherent culture protocol (Example 1).

4.0 Notes:

Note 1: Charcoal-stripped FBS must be heat-inactivated prior to use. Heat in 55° C. water bath for 60 min. Heat-inactivated charcoal-stripped FBS can be aliquotted and stored at −20° C.

Note 2: Matrigel must remain ≦4° C. at all times until use to prevent polymerization. It is recommend to place the Matrigel in 4° C. refrigerator overnight to thaw and keeping it on ice until it is added to media. Unused Matrigel can be refrozen, but avoid multiple freeze-thaw cycles.

Note 3: It is recommend to culture without antibiotics, but antibiotics can be added during initial culturing period or if there is increased concern for contamination from other sources.

Note 4: Shaking the tube periodically to redistribute bladder tissue is helpful.

Note 5: If fewer than 75,000 cells are obtained after organoid dissociation, resuspend in 500 μL media. Lower density plating will take longer to reach confluence but can be successfully cultured with as few as 15,000 cells. If fewer than 15,000 cells are obtained, we recommend replating in organoid culture (5,000 cells/100 μL media per well in low-attachment 96-well plate).

### Example 3—an Individualized Approach to Bladder Cancer Treatment Using Patient-Derived Cell Lines to Predict Response to Chemotherapeutic Agents

Introduction:

Chemotherapy (both intravesical and systemic) can reduce the risk of recurrence and progression in various stages of bladder cancer. However, recurrence after treatment failure is associated with an increased risk of progression. There are currently no established methods for predicting patient-specific responses to treatment prior to drug selection. Described herein is the development of a new protocol for efficient establishment of cell lines from primary human bladder tumors, which enables in vitro drug sensitivity assays using chemotherapeutic agents.

Methods:

Using a tissue acquisition protocol, informed consent was obtained prior to specimen acquisition for all samples. Specimens were obtained during standard transurethral resection of papillary bladder tumors. Following generation of a single-cell suspension, epithelial cells were isolated using immunomagnetic cell separation and used for establishment of adherent cell cultures using a new protocol. Immunohistochemistry was performed on parental tissue as well as cultured cells to confirm that the urothelial cancer phenotype was maintained during serial passaging. For sensitivity assays, cultured cells were passaged and treated with chemotherapeutic agents, followed by assessment of cell viability using MTT assays.

Results:

To date, seven specimens from patients with papillary urothelial carcinoma have been obtained, resulting in the establishment of six independent adherent cell lines. All established lines have been serially passaged (as high as P10) without significant decline in growth rate, and maintained expression of CK7, uroplakin III, p53, and Ki67 in patterns similar to parental tissue. Cells from line #7 were treated with mitomycin C, docetaxel, gemcitabine, and rapamycin at three different equivalent concentrations, resulting in a unique sensitivity profile that was reproduced in a replicate experiment performed at a subsequent passage.

Conclusions:

A new protocol has been established for culture and rapid expansion of primary cells from human bladder tumors for assays of drug response. Ultimately, this approach can provide a basis for the design of patient-specific therapeutic regimens for bladder cancer.

### Example 4: An Individualized Approach to Bladder Cancer Treatment Using Patient-Derived Cell Lines to Predict Response to Chemotherapeutic Agents

Introduction:

Intravesical therapy (when antineoplastic agents are instilled directly into the bladder via urethral catheter) can reduce the risk of recurrence after standard endoscopic resection of bladder tumors. Many patients will not respond to intravesical treatment, and each recurrence is associated with an increased risk of progression. There are currently no established methods for predicting patient-specific responses to intravesical treatments prior to drug selection. Previous studies have had limited success in establishing patient-derived cell lines from primary bladder tumor specimens due to short-term culture (1-7 days), limited efficiency (31-78% success rates across studies), samples often taken from cystectomy specimens (requires removal of entire bladder; not useful for testing intravesical agents). Ideal scenario would be for rapid sensitivity testing prior to initiating adjuvant intravesical therapy (typically 2-6 weeks after tumor resection).

Described herein is the establishment of a protocol for the rapid and efficient establishment of cell lines from primary human bladder tumors obtained during routine endoscopic biopsy or resection. These patient-derived cell lines can be used to perform in vitro drug sensitivity assays.

Results:

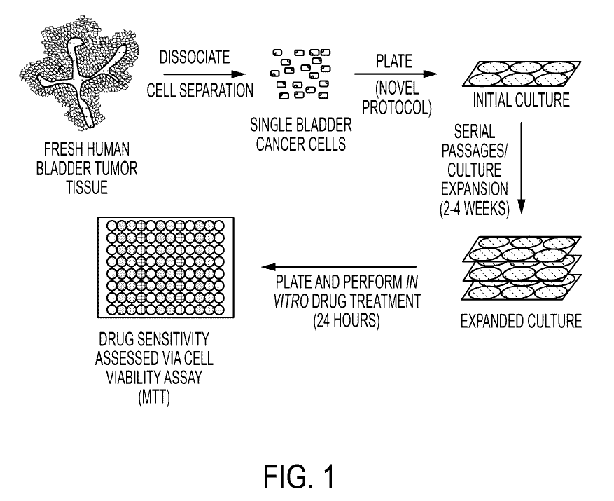

FIG. 1 shows a schematic of the method for establishing patient-specific bladder cancer cell cultures for drug sensitivity testing. Table 1 shows a summary of patient-derived bladder cancer cell lines.

TABLE 1
Summary of patient-derived bladder cancer cell lines

| Line # | Tumor | Tumor | Sample Weight | Established | Number of |
|---|---|---|---|---|---|
| (gender) | Grade | Stage | (grams) | Culture? | Passages |
| 1(M) | High/Low | Ta | 2.20 | Yes | 5 |
| 2(F) | Low | Ta | 0.10 | Yes | 3 |
| 3(M) | High | Ta | 0.04 | Yes | 7 |
| 4(F) | High | T1 | 0.08 | No | (NA) |
| 5(F) | Low | Ta | 0.02 | Yes | 9 |
| 6(F) | High | Ta | 1.75 | Yes | 10* |
| 7(M) | High/Low | T1 | 0.50 | Yes | 17 |
| 8(M) | Low | Ta | 0.04 | Yes | 3 |
| 9(M) | High | T2 | 0.51 | Yes | 11* |

(*denotes actively growing lines)

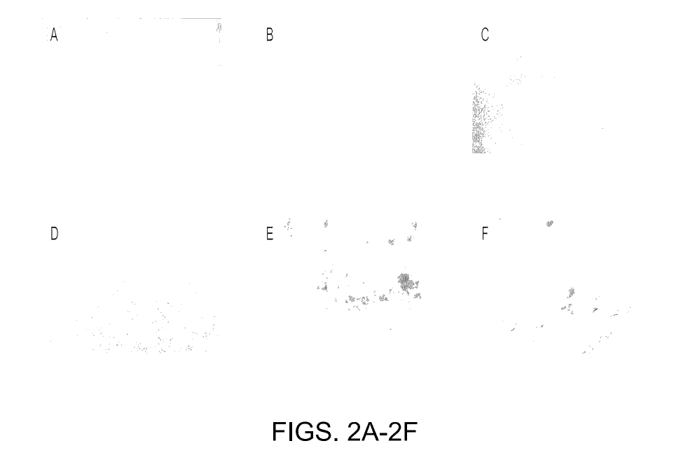

FIGS. 2A-F shows patient-derived bladder cancer cell lines in culture. FIG. 2A: Single cells are seen on day 1 of adherent culture. FIG. 2B: Small colonies are seen by day 6. FIG. 2C: Large colonies with moderate confluence seen on day 12. FIG. 2D: Colonies are seen on day 5 after two passages. FIGS. 2E-F: Spherical “organoids” form when cells are grown in 3-dimensional floating culture.

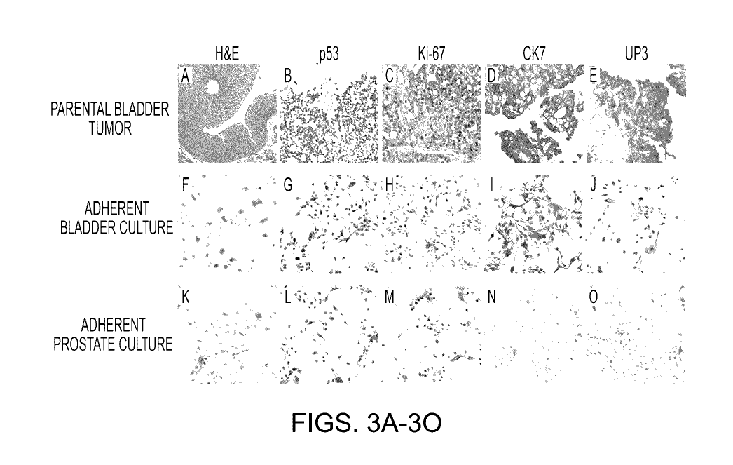

FIGS. 3A-O shows immunohistochemical analysis of patient-derived cell lines. FIGS. 3A-E: Histological analysis of parental tumor tissue from Line #7 using H&E (FIG. 3A), p53 (FIG. 3B), Ki-67 (FIG. 3C), cytokeratin 7 (FIG. 3D), and uroplakin III (FIG. 3E) are all consistent with high-grade urothelial carcinoma. FIGS. 3F-J: Identical staining performed on fixed adherent cells grown on slides show similar staining pattern as parental tissue. FIGS. 3K-O: Identical staining on cultured human prostate cancer cells shows similar p53 and Ki-67 staining but no cytokeratin 7 or uroplakin III staining.

TABLE 2
Drugs used for sensitivity assays. † denotes the maximum in vitro
concentration based on drug's maximum solubility in DMSO (with 0.5% DMSO in final
culture media). ‡ denotes 1X, 10X, and 100X concentrations represent equivalent dilutions of
in vivo concentrations across different agents. ** denotes the Rapamycin in vivo
concentration based on mouse studies.

| | | | | | | | | In vivo to | |
|---|---|---|---|---|---|---|---|---|---|
| | Standard | | | | | | | max in | In vivo |
| | human | In vivo | | Maximum | | | | vitro | to 1X |
| | intravesical | intravesical | | in vitro | 1X | 10X | 100X | dilution | dilution |
| Agent | dosing | concentration | | conc.† | conc.‡ | conc.‡ | conc.‡ | ratio | ratio |
| Docetaxel | 75 mg/100 mL | 0.75 | mg/mL | 6.19 μM | 6.19 μM | 619 nM | 61.9 nM | 1:150 | 1:150 |
| | | (928 | μM) | | | | | | |
| Gemcitabine | 2 g/100 mL | 20 | mg/mL | 950 μM | 507 μM | 50.7 μM | 5.07 μM | 1:80.0 | 1:150 |
| | | (76.0 | mM) | | | | | | |
| Mitomycin | 40 mg/20 mL | 2 | mg/mL | 150 μM | 39.9 μM | 3.99 μM | 399 nM | 1:39.9 | 1:150 |
| | | (5.98 | mM) | | | | | | |
| Rapamycin | ** | 15 | mg/mL | 547 μM | 109 μM | 10.9 μM | 1.09 μM | 1:30.0 | 1:150 |
| | | (16.4 | mM) | | | | | | |

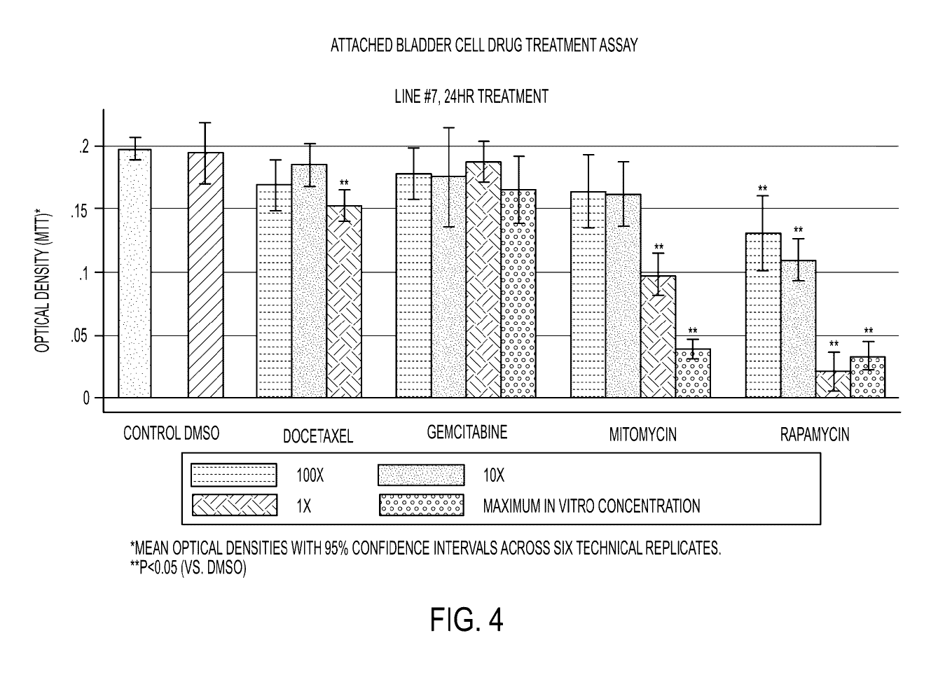

Table 2 shows the drugs used for sensitivity assays. FIG. 4 shows the drug sensitivity profile for line #7. Drug sensitivity was performed after 24-hour drug exposure followed by MTT proliferation assay. Optical density from MTT assay is proportional to viable cells present. Mean optical densities with 95% confidence intervals for six technical replicates of each drug dilution are shown. Statistical comparisons were made between DMSO only (pink bar) and each drug dilution.

Conclusion:

A new protocol has been established for the culturing and rapid expansion of primary cells from human bladder tumors with high efficiency (89% success rate). These cell lines maintain immunohistochemical staining patterns similar to parental tissue and consistent with bladder cancer. Rapid expansion allows drug sensitivity assays to be performed 2-4 weeks after initial biopsy (i.e. prior to initiating adjuvant intravesical treatment). This approach provides a basis for the design of patient-specific therapeutic regimens in bladder cancer.

### Example 5: Method for Growing Bladder Organoid

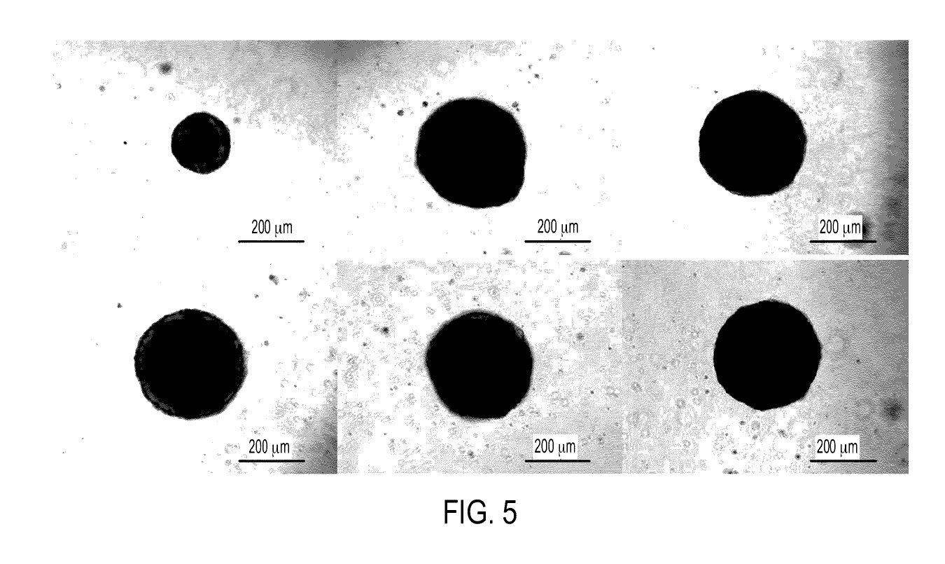

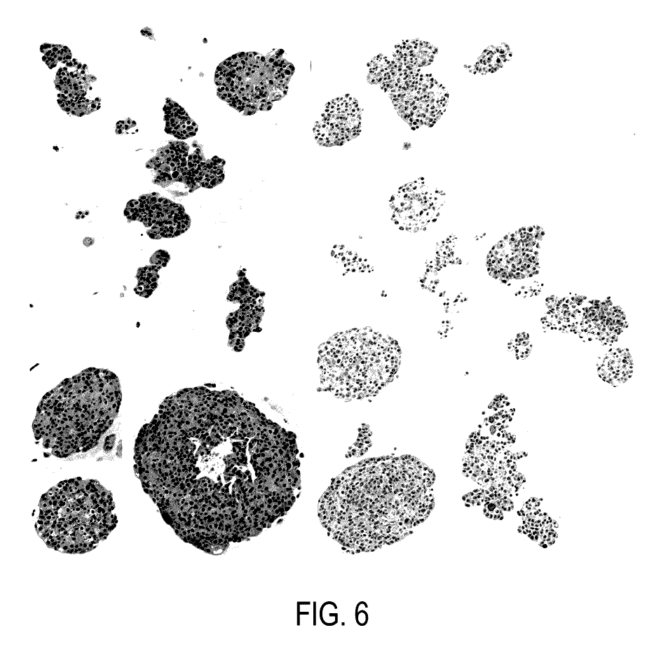

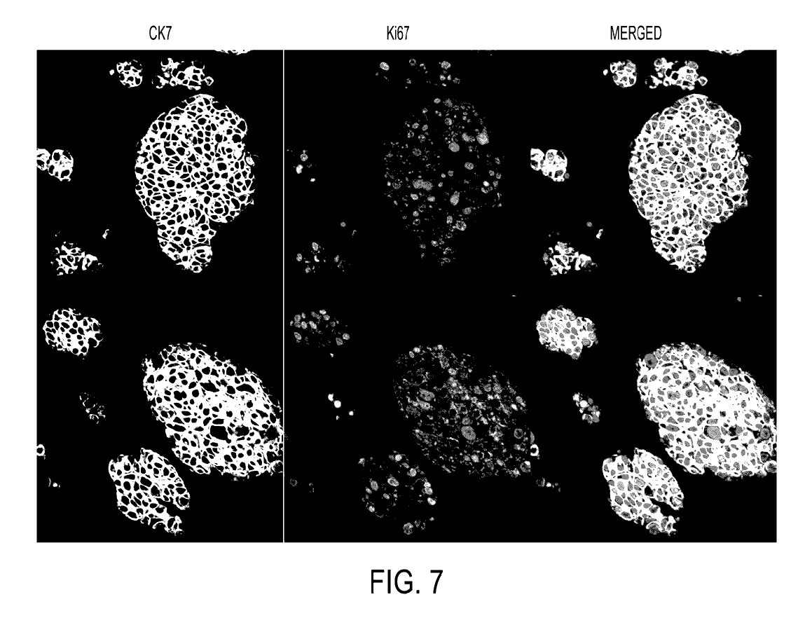

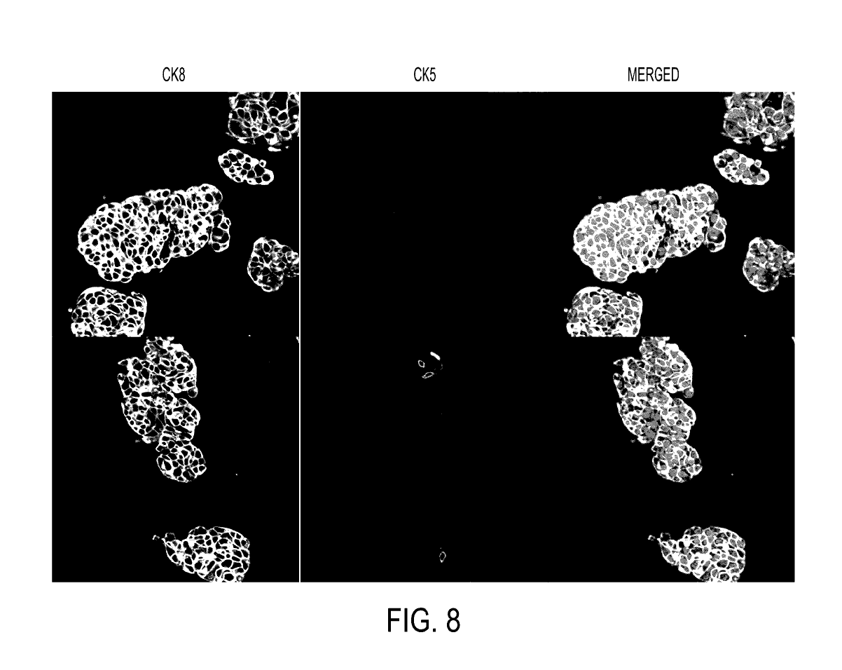

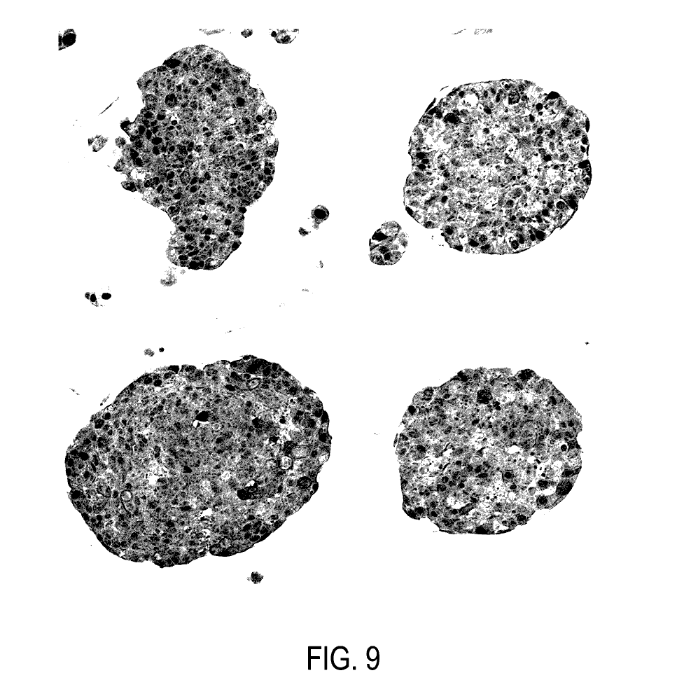

Described herein is methodology for generating bladder organoids that uses culture embedded in Matrigel (Matrigel embedding method), rather than floating on top of a Matrigel layer (Matrigel floating method). Several new bladder tumor organoid lines have been established using the embedding methodology (“MaB” series), as well as the Matrigel floating methodology described in Example 2 (“LaB” series). The Matrigel embedding methods improves the passaging and survival of the organoid lines. A summary of the MaB and LaB cell lines is presented in Table 3. Characterization of bladder tumor organoid line MaB22 is shown in FIGS. 5-9. Immunostaining of MaB22 confirms the tumor content, these organoids are uniformly cytokeratin 7 (CK7) positive (FIG. 7) and have nuclear p53 immunostaining (FIG. 9). These properties are characteristic of bladder tumors.

TABLE 3
Summary of MaB and LaB cell lines

| | | | | Established | | | | |
|---|---|---|---|---|---|---|---|---|
| | | Tumor | | Embedded | Number of | Parental | Parent | |
| Specimen | Cysview Use | Grade | Tumor Stage | Culture | Passages | Tissue Block | Tissue DNA | Characterization |
| LaB4 | no | Hg | Ta | yes | 7 | available | | non-embedded organoid/ |
| | | | | | | | | currently culturing |
| Lab7 | no | Hg | Ta | yes | 2 | available | available | non-embedded organoid/ |
| | | | | | | | | currently culturing |
| Lab11 | no | Hg | T2 | growing slowly | 4 | available | | non-embedded organoid/ |
| | | | | | | | | currently culturing |
| MaB19 | no | Hg | Ta | yes | 7* | available | | CWC - IF Staining |
| MaB22 | no | Hg | Tl/Cis | yes | 3* | available | | button done/processing |
| MaB25 | no | Hg | Tl/Cis | growing slowly | 2* | available | | culturing |
| MaB26 | no | Lg | Ta | yes | 2* | available | | culturing |

Matrigel Embedding Method Protocol

1. The bladder tumors was resected from patients and followed by washing in Gentamicin for 5 minutes.

2. The tissue was then minced with scissors, and followed by incubation in Collagenase/Hyaluronidase solution for 1 hour at 37 C. Collagenase/Hyaluronidase solution is prepared by 1 part of 10× Collagenase/Hyaluronidase solution (Stem Cell Technologies, Cat. #07912) with 9 part of Hepatocyte medium supplemented with 5% FBS).

3. The tissue was incubated in TrypLE solution (Life Technologies, Cat #12605) for 20-30 minutes at 37 C to dissociate the cells into clusters form.

4. The cell clusters were then treated with 0.1 mg/mL DNase I (Prepared from 1 mg/mL DNase I, Cat #07900) in hepatocyte medium.

5. The cell clusters were then mixed with 0.5 ml of a 60:40 Matrigel:Hepatocyte medium solution, and plated onto the well of a 6-well plate. It is important that the plate is pre-coated with a rinse of 60:40 Matrigel:Hepatocyte solution and followed by the incubation of the precoated plate at 37 C for at least 30 minutes prior to use.

6. The embedded cell clusters in Matrigel solution was allowed to solidify in 37 C incubator for 30 minutes. Warmed complete hepatocyte medium (supplemented with EGF/Glutamax/5% Heat-inactivated FBS) was then carefully applied to the solidified matrigel from the edge of the well.

7. Medium change was done for every 3-4 days until the organoids were ready for passage.

8. To passage the organoid, 5 mg/ml Dispase was added directly into the well to bring the final concentration of Dispase to 1 mg/ml (For example, if there is 1.2 ml of medium in the well, 0.3 ml of 5 mg/ml Dispase will be added). The plate was incubated at 37 C for 30 minutes.

9. After 30 minutes, the Matrigel should be dissolved, and the organoids were released from the embedded Matrigel. The organoids in Dispase solution was further diluted in HBSS 2% FBS (1.5 ml of Organoids in Dispase solution+7.5 HBSS).

10. The organoids were then washed with 1 change of 1×PBS. After pelleting the organoids, PBS was removed and TrypLE was applied and mixed well with the cell pellet. Dissociation with TypLE should be done within 1-2 minutes at RT (Prolonged incubation of TypLE will lead to dissociation of organoid into single cells, which consequently causes reduced cell viability and growth).

11. The cell clusters were then replated as stated in Step 5 and 6.

Collagen Embedded Method:

The cell clusters could also be mixed and embedded with 0.5 ml of a collagen mixture solution—9 Part of Collagen I, High Concentration, Rat tail, Cat. #354249 and 1 Part of setting solution formulated as follows: 10×EBSS—100 ml; Sodium bicarbonate—2.45 g; 1M NaOH—7.5 ml; Sterile milliQ water—42.5 ml. It is important that the plate is pre-coated with 200 ul of collagen mixture solution and followed by the incubation of the precoated plate at 37° C. for at least 30 minutes prior to use. In addition, collagen mixture will only be prepared prior to used.

Lastly, the embedded cell clusters in Collagen mixture solution can be allowed to solidify in 37° C. incubator for 30 minutes. Warmed complete hepatocyte medium (supplemented with EGF/Glutamax/5% Heat-inactivated FBS) can then be carefully applied to the solidified collagen from the edge of the well.

In order to passage the cell clusters embedded in collagen, medium can be replaced with collagenase solution (Sigma, C9697—Stock at 25 mg/ml prepared in HBSS supplemented with 2% FBS) at 0.25 mg/ml in hepatocyte medium for 30 minutes at 37° C. Collagen can be digested and the organoids can be released from the collagen.

11. The cell clusters were then replated as stated in Step 10 and 11 above.

### Example 6: Establishment and Analysis of Patient-Derived Bladder Cancer Organoid Lines

To establish bladder cancer organoid lines, a novel protocol for the dissociation and three-dimensional culture of fresh bladder tumor tissue has been developed. These conditions are based upon those that we previously established for mouse and human prostate organoids [84], and were guided by the importance of Matrigel in three-dimensional culture of prostate and mammary epithelium [99, 100], hepatocyte medium for prostate epithelial cell culture [101], and ROCK inhibitor to improve the survival of dissociated epithelial cells [102-104]. Importantly, the protocol described herein differs from the conditions utilized by the Clevers lab to culture epithelial organoids from a range of tissues [80, 81, 105-109], and is functionally distinct in being more favorable for the culture of prostate luminal epithelial cells.

Using these organoid culture conditions, fresh bladder tumor tissue obtained by transurethral resection (TUR) was dissociated and cultured. Currently, organoid lines are established with an efficiency of approximately 25-30%, and to date have successfully generated 14 independent patient-derived organoid lines. These lines have been propagated for at least three passages, and have been successfully cryopreserved, allowing their long-term storage and retrieval. In addition, clinical records about tumor pathology and patient treatment have been maintained, and are summarized in Table 4. For example, 8/14 patients received prior treatment, either intravesical or systemic, while the remaining 6/15 patients were treatment-naïve Table 4. Notably, two of the organoid lines (MaB30 and MaB30-2) were established from chronologically distinct lesions from the same patient whose bladder cancer that recurred after 13 months following treatment with intravesical BCG and mitomycin C.

TABLE 4
Summary of patient-derived organoid lines.

| | | | | | Prior | | |
|---|---|---|---|---|---|---|---|
| | | | | Prior Intravesical | Systemic | Passage | Corresponding |
| Specimen | Grade | Stage | Sex | Therapy | Therapy | number | xenograft |
| MaB19 | Hg | Ta | F | Docetaxel | None | 12 | No |
| MaB28 | Lg/Hg | T2 | M | None | None | 13 | Yes |
| MaB30 | Lg/Hg | T1 | M | Docetaxel | None | 13 | Yes |
| MaB33 | Hg | T2 | F | BCG, BCG-IFN | None | 11 | Yes |
| JuB3 | Hg | T1 + CIS | M | None | None | 20 | Yes |
| SuB2 | Hg | T1 + CIS | M | MMC, BCG | None | 10 | Yes |
| SuB4 | Lg/Hg | Ta | M | MMC, BCG | None | 4 | — |
| MaB30-2 | Lg/Hg | Ta | M | Docetaxel, BCG, | None | 9 | Yes |
| | | | | MMC | | | |
| SuB6 | Lg/Hg | Ta | F | None | None | 5 | No |
| SuB9 | Lg/Hg | T1 | M | None | None | 6 | Yes |
| SuB10 | Lg | Ta | M | MMC, BCG | None | 3 | — |
| SuB11 | Lg | Ta | F | None | None | 3 | — |
| SuB12 | Lg | Ta | M | None | None | 5 | — |
| SuB13 | Hg | T2 | M | None | Gem, Cis | 4 | — |
| SuB15 | Hg | Ta + CIS | F | None | None | 2 | — |

Abbreviations:
BCG, Bacillus Calmette-Guérin treatment;
Cis, cisplatin;
CIS, carcinoma in situ;
Gem, gemcitabine;
Hg, high-grade;
IFN, interferon,
Lg, low-grade;
MMC, mitomycin C;
—, not determined.

Of particular note, 4/15 organoid lines were established from female patients, which correlates with the three-fold higher incidence of bladder cancer in men [32]. Furthermore, one organoid line (MaB30) was derived from an African-American patient, while another line (JuB3) was established from a Hispanic patient (2/15 total), which is consistent with the overall demographics of the patient population at the medical center where the samples were collected. Thus, the continued generation of patient-derived organoid lines may provide a basis for disparities research in bladder cancer.

It is noted that samples obtained by TUR are inherently biased towards non-invasive bladder tumors, since these cases represent the more prevalent form of bladder cancer. Nonetheless, since patients with muscle invasive bladder cancer also undergo cystoscopy, to date, three organoid lines have been generated for muscle invasive disease, corresponding to MaB28, MaB33, and SuB13 (Table 4). As noted previously, non-muscle invasive bladder cancer is clinically important as it is often associated with considerable morbidity and expensive long-term treatment [2, 26]. However, since the broad objective is to generate patient-derived organoid lines that are representative of the full spectrum of bladder cancer, patient-derived models will also be established from patients at alternative medical centers, which have a patient population that is biased towards more advanced cases of bladder cancer.

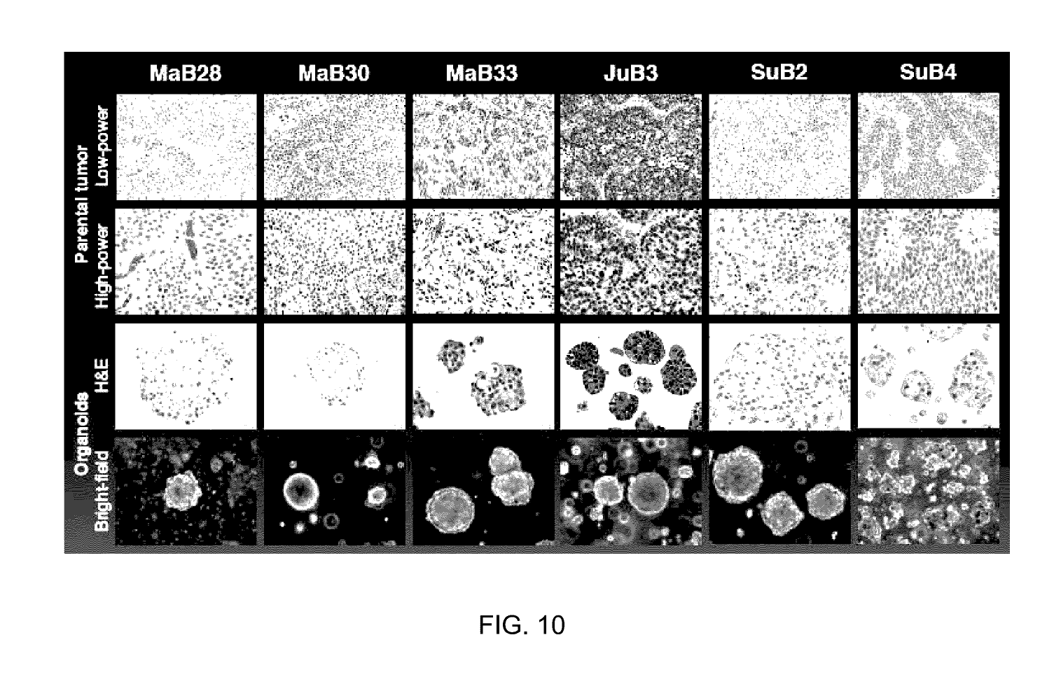

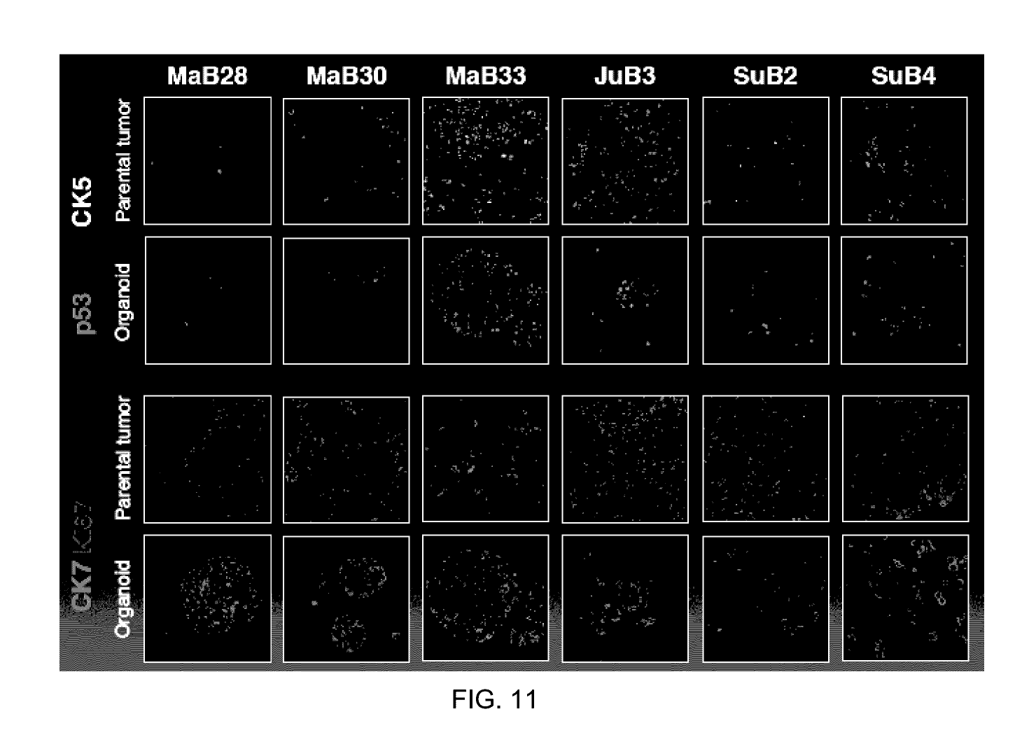

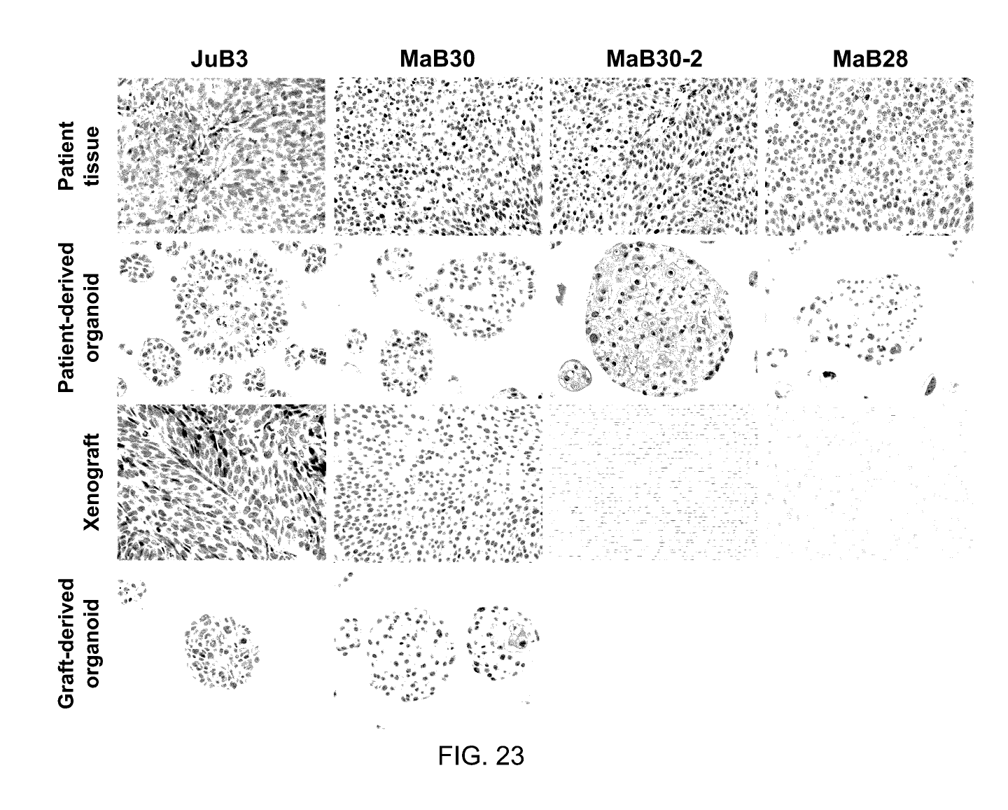

To determine whether the histological phenotypes of the patient-derived organoid lines resembled their corresponding parental tumors, hematoxylin-eosin (H&E) staining of paraffin sections was performed. Light-microscopic examination of the H&E-stained slides showed that the histopathological features of the patient-derived organoid lines were identical to those of their corresponding parental tumors (FIG. 10; see also FIG. 23). This analysis indicates the presence of strong phenotypic concordance between the parental tumors and corresponding organoids.

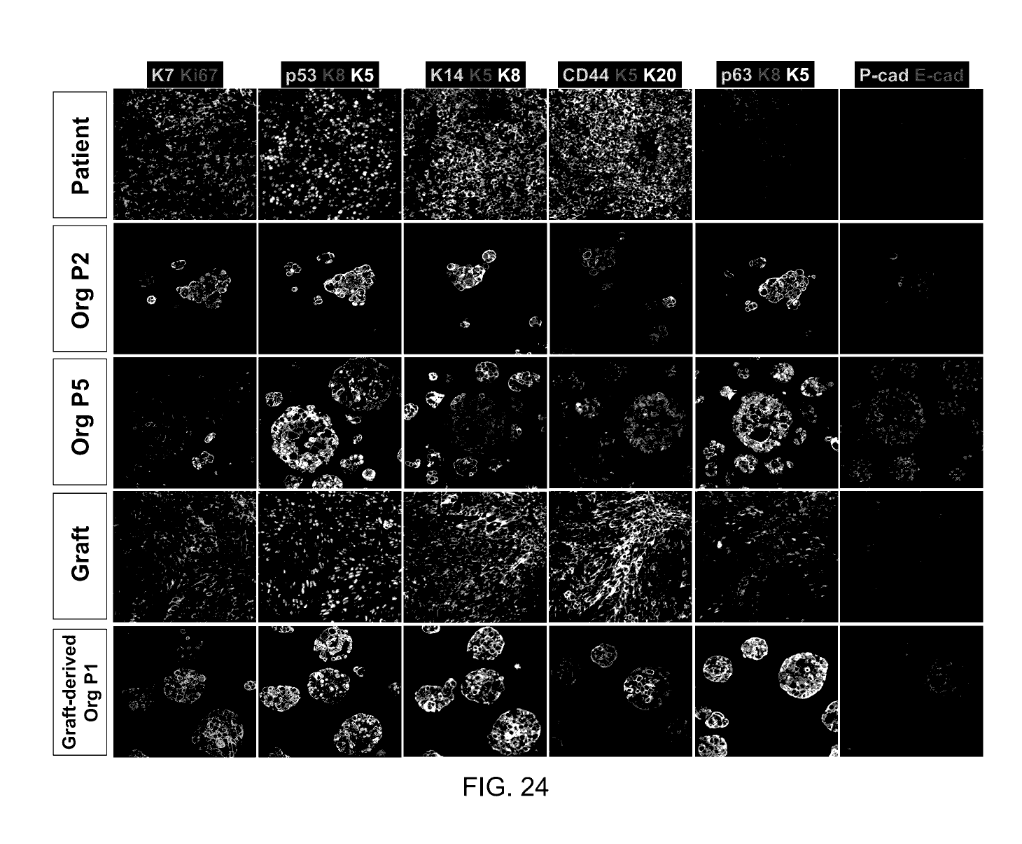

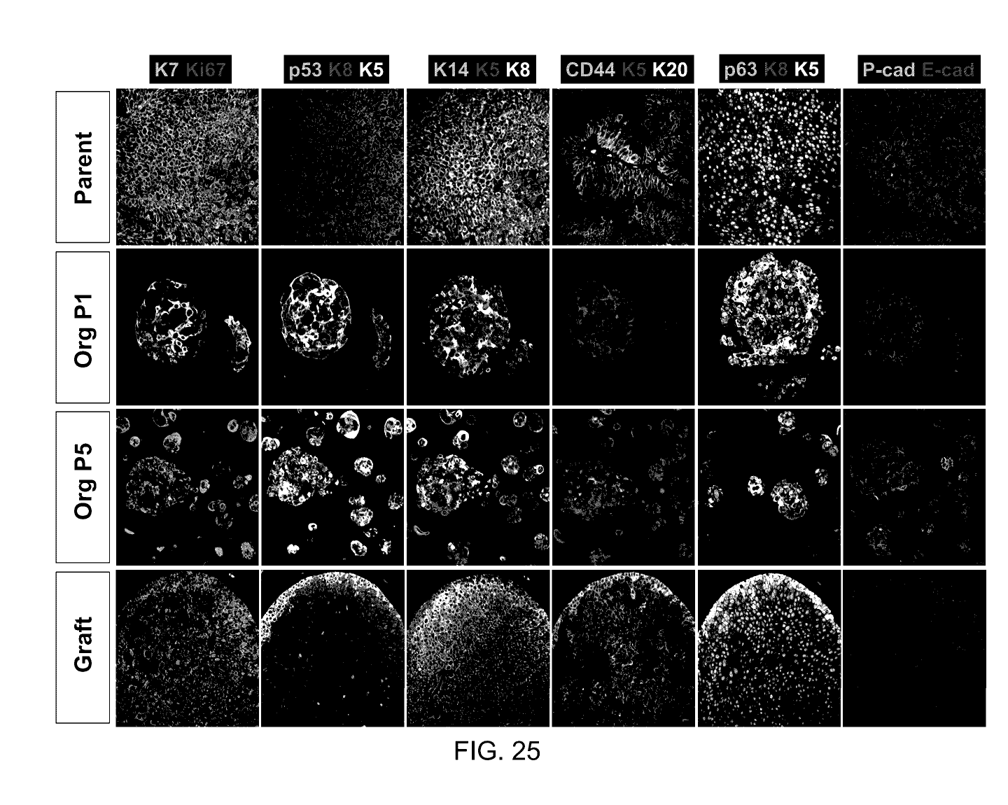

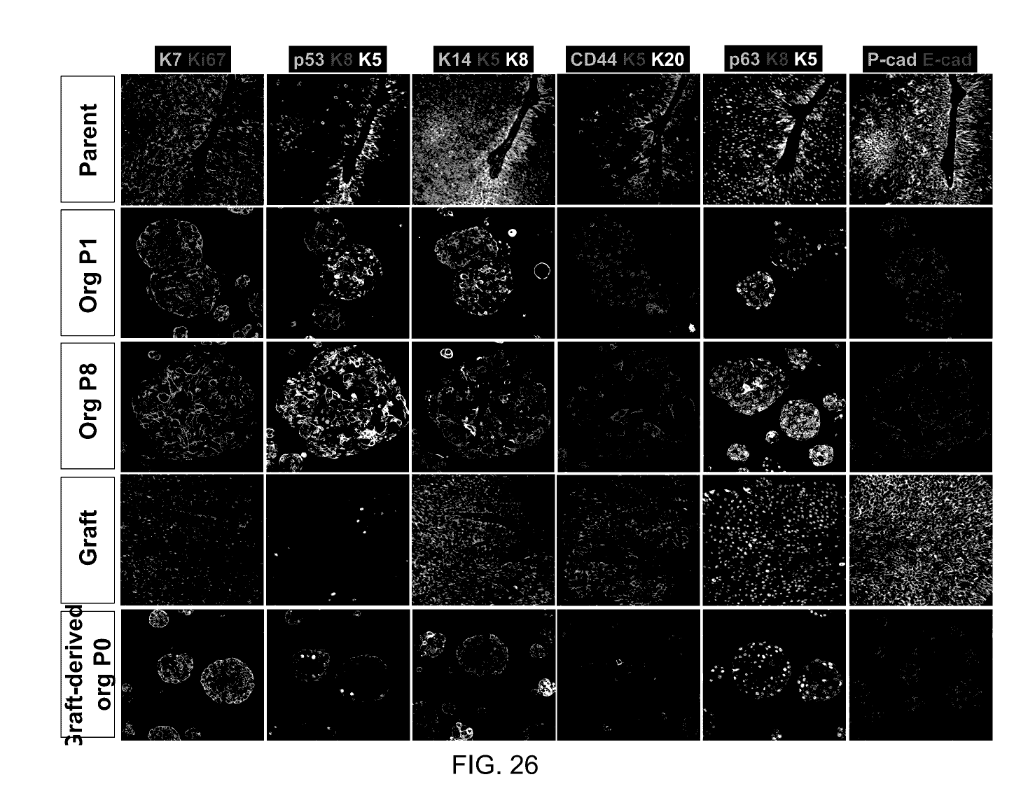

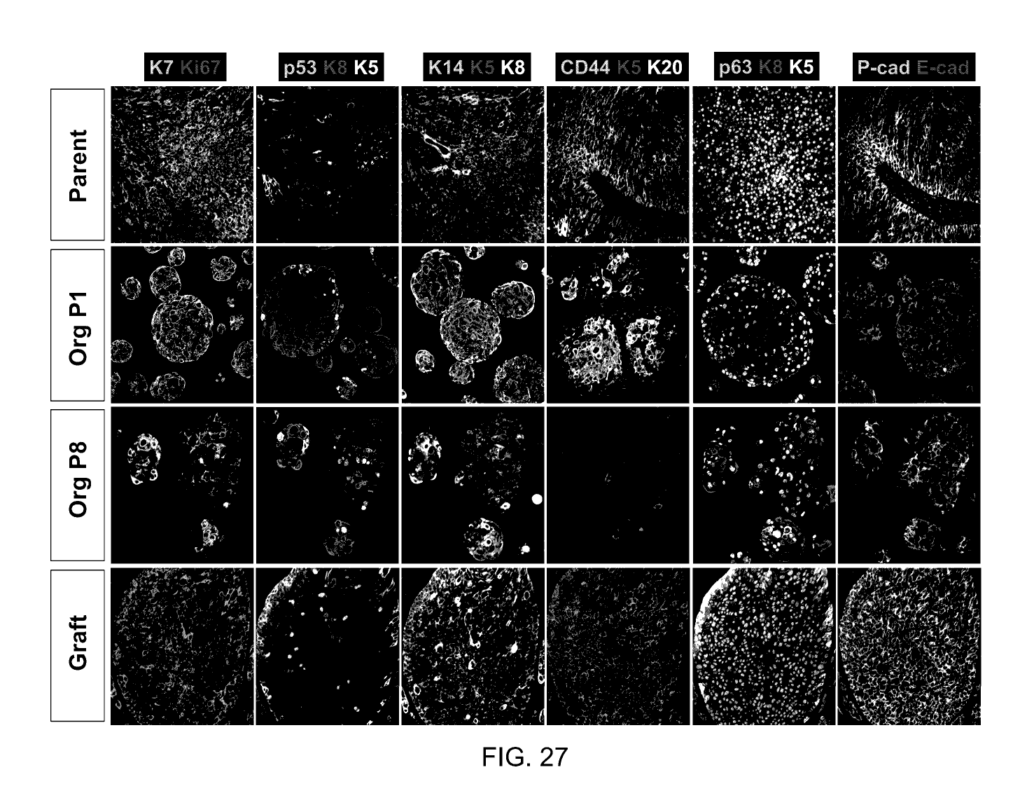

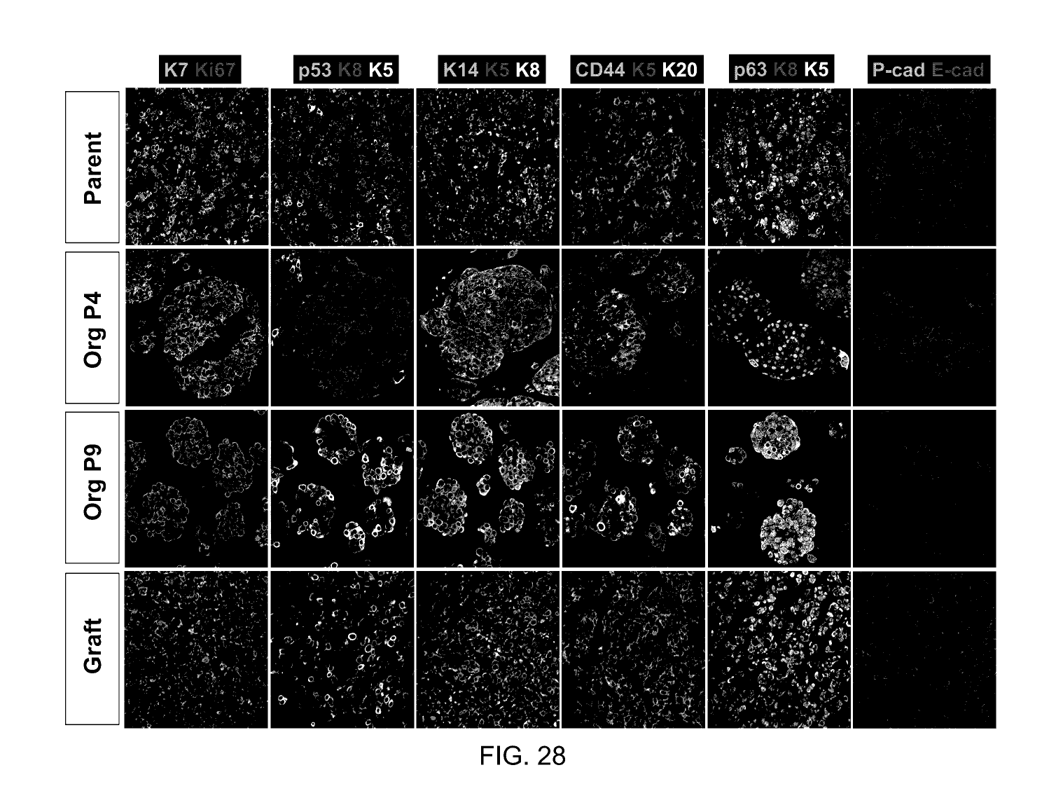

Next, analyses of marker expression was performed in six independent patient-derived organoid lines by immunofluorescence (FIG. 11; see also FIGS. 24-28). For these analyses, immunostaining for the basal epithelial marker cytokeratin 5 (CK5), the luminal marker cytokeratin 8 (CK8), and CK7, which is strongly expressed by all urothelial cells was performed. Expression of p53 was also examined to detect potential mutations in TP53, which would lead to increased nuclear localization, as well as for Ki67 to assess cellular proliferation. It was found that two of these lines (MaB33 and JuB3) express nuclear p53 protein, suggesting that these lines contain TP53 mutations (FIG. 11). Furthermore, the analyses showed that most of the organoid lines display strong widespread expression of the luminal marker CK8, as well as the urothelial marker CK7, consistent with the phenotype of their corresponding parental tumors. However, a small percentage of cells in two of the organoid lines (MaB30 and SuB2) showed expression of the basal marker CK5, which is also observed in the corresponding parental tumors. This finding suggests that there is phenotypic heterogeneity in the parental tumor that is retained in the corresponding organoid line.

To analyze the genomic alterations in these patient-derived organoid lines, targeted exome sequencing was performed using the MSK-IMPACT platform [95]. For these analyses, sequencing of the organoid line was performed together with the corresponding parental tumor as well as normal blood from the same patient. The output of these targeted exome sequencing analyses was then analyzed using a customized bioinformatic pipeline, and visualized through the cBioPortal for Cancer Genomics, a comprehensive web-based resource for interactive exploration of multidimensional cancer genomics data [110, 111]. Data visualization through cBioPortal integrates somatic mutations and DNA copy-number changes (such as focal amplifications or homozygous deletions), as well as gene expression and methylation data, when available.

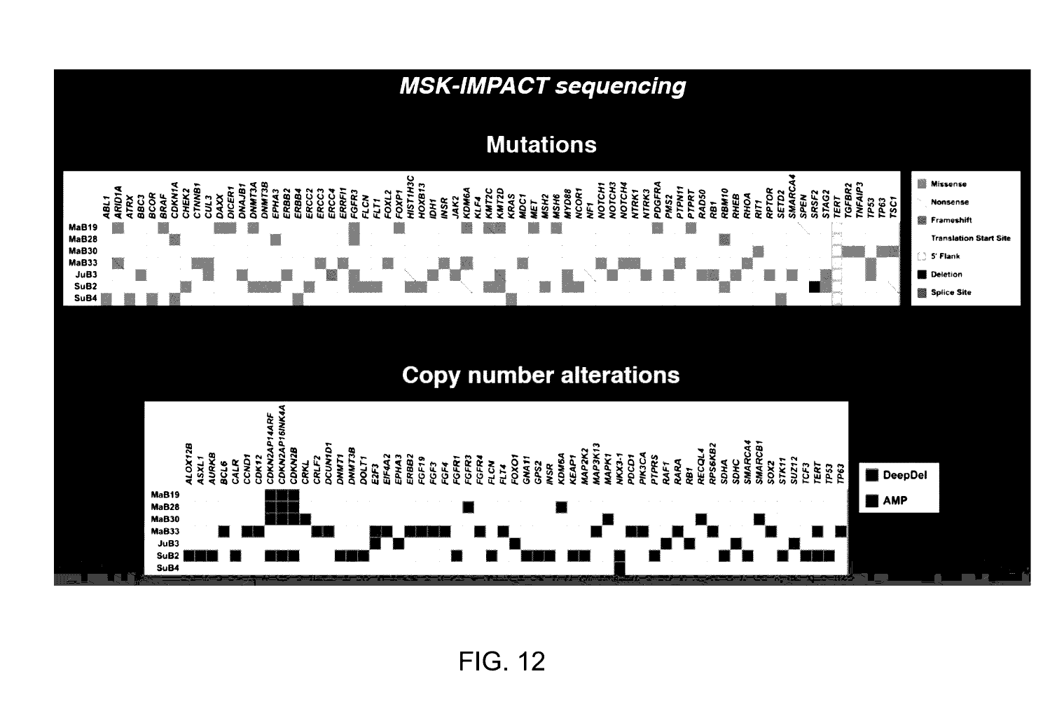

These sequencing analyses identified numerous genomic alterations in these patient-derived organoid lines, including mutations in ARID1A, ERRC2, FGFR3, KDM6A, RB1, and TP53 (FIG. 12). Furthermore, the TP53 mutations identified in the MaB33 and JuB3 lines were consistent with the observed nuclear p53 immunostaining (FIG. 11). Of particular note, an ERBB2 mutation was identified in the JuB3 organoid line, and a mutation in KRAS in the SuB4 line. Importantly, the mutational profiles observed in these patient-derived organoid lines are characteristic of human bladder cancer, as described in multiple large-scale studies [22, 57-60], indicating that these lines are highly representative of the genomic spectrum of the disease.

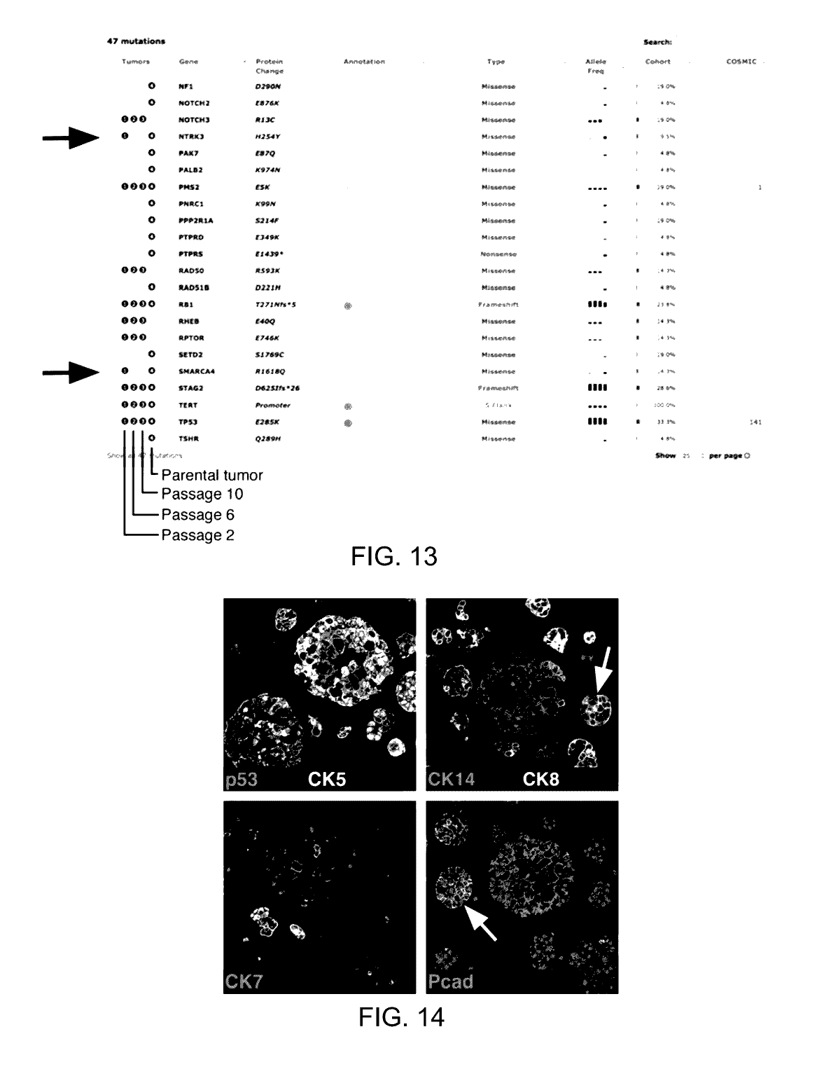

During serial passaging of the JuB3 line, a subtle alteration of organoid morphology and increased growth rate between passages 2 and 10 was noticed. Consequently, organoids from this line were analyzed at different passages by targeted exome sequencing, and by immunostaining of markers. The summary of these sequence analyses as shown in cBioPortal revealed that the organoid population had changed its mutational profile between passages 2 and 6 (FIG. 13). Thus, some mutations were observed in organoids at passages 2, 6, and 10, as well as in the parental tumor, including mutations in RB1, STAG2, and TP53. However, other mutations were only found at passage 2 and in the parental tumor, but were not detected at passages 6 and 10, such as mutations in NTRK3 and SMARCA4 (FIG. 13, arrows). Notably, the mutations in NTRK3 and SMARCA4 were clearly present at subclonal allele frequencies at both passage 2 and in the parental tumor.

Consistent with these molecular profiles, marker expression in JuB3 organoids at passage 6 was also examined (FIG. 14). It was found that, unlike at passage 2 (see FIG. 11), expression of the basal cytokeratin CK5 was up-regulated, and expression of the luminal cytokeratin CK8 was down-regulated; expression of the urothelial marker CK7 was also down-regulated. Furthermore, immunostaining of the organoid population revealed considerable phenotypic heterogeneity, with a subpopulation of organoids displaying up-regulation of the basal cytokeratin CK14 as well as down-regulation of E-cadherin and up-regulation of P-cadherin, perhaps consistent with emergence of a mesenchymal phenotype [112]. Consequently, these analyses of JuB3 serial passages suggest that processes of clonal evolution can affect tumor phenotype in organoid culture. These findings support the feasibility of studies of clonal evolution in organoids and xenografts.

Generation of Matched Pairs of Patient-Derived Organoid and Xenograft Lines

For the studies of tumor evolution and drug response, it is essential to analyze organoid and xenograft lines that are derived from the same patient tumor. Therefore, pilot studies have been performed to demonstrate the feasibility of generating matched pairs of patient-derived organoid and xenograft lines by generating xenografts from organoids, and vice versa, organoids from xenografts. As a result, analyses of matched patient-derived organoid and xenograft lines from the identical starting point can be performed.

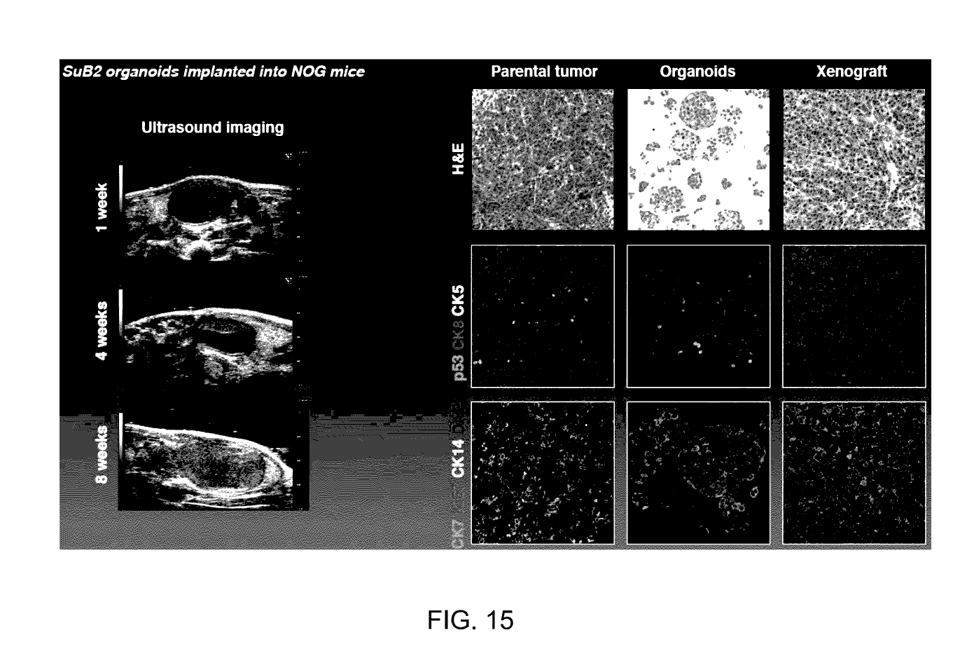

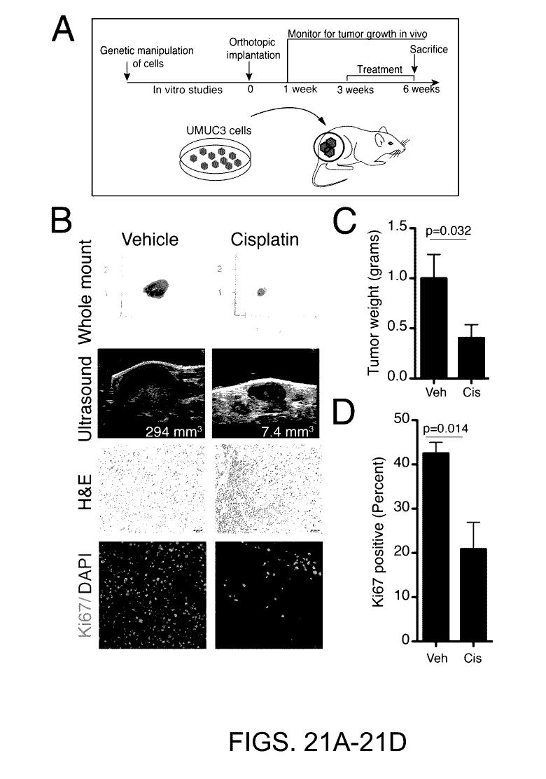

To generate xenografts from patient-derived organoids, we have used the orthotopic grafting methodology (see FIGS. 21A-B). Using ultrasound-guided implantation, organoids were implanted into the bladder wall of NOG immunodeficient mice, and then longitudinal analyses of their growth was performed over two months by three-dimensional ultrasound imaging (FIG. 15, left). This preliminary experiment showed that engraftment of organoids occurs with high efficiency, as 7 out of 9 (78%) organoid lines implanted resulted in successful xenografts (Table 4). Analyses of the resulting xenografts demonstrated that their histology resembled that of the corresponding organoid line and parental tumor (FIG. 15, right). Notably, it was observed that a subpopulation of CK5-positive cells that is observed in the parental tumor is also present during organoid passaging, and is still found in the subsequent xenograft, suggesting that phenotypic heterogeneity can be retained in xenografts established from patient-derived organoid lines.

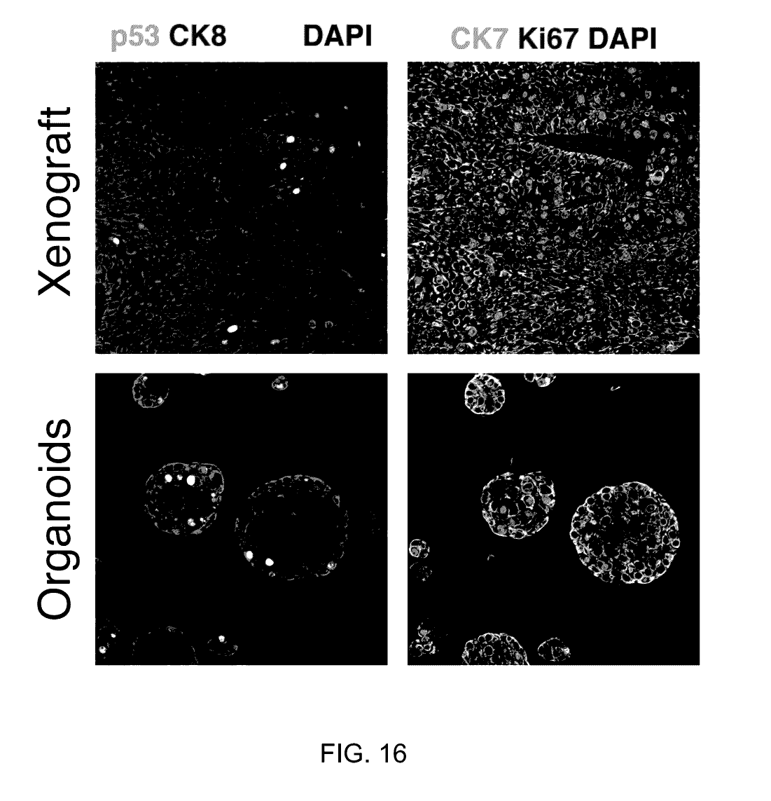

The opposite conversion by generating organoids from patient-derived xenografts has been successfully performed. Using a protocol similar to the initial tissue dissociation of tumor tissue to establish organoid lines, organoids from 2 out of the 2 xenograft lines attempted were successfully generated. Immunofluorescence analyses of these organoids showed their phenotypic similarity to the starting xenograft tissue (FIG. 16). Thus, this data suggests that organoids and xenografts can be successfully interconverted with high efficiency in both directions.

Analysis of Drug Response in Patient-Derived Organoids

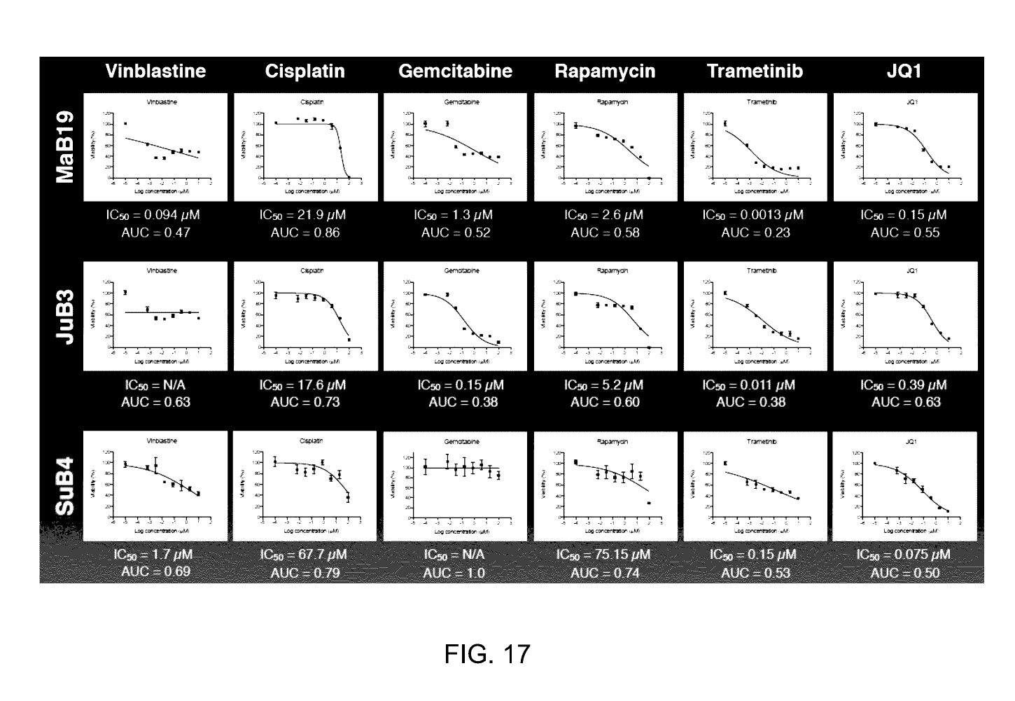

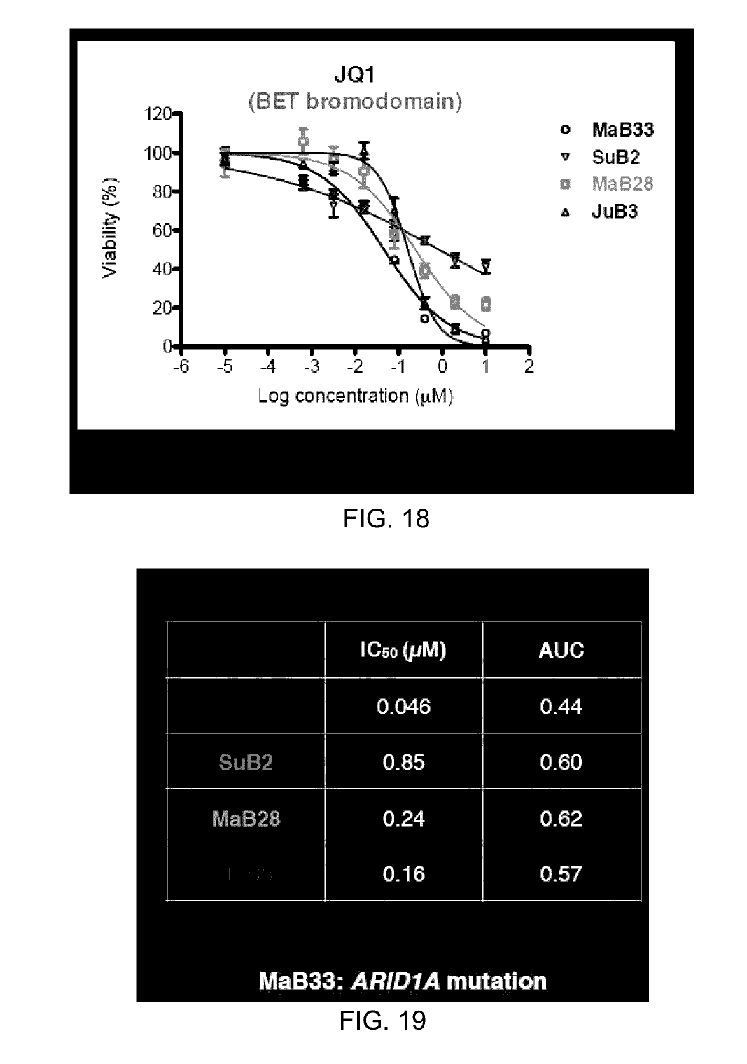

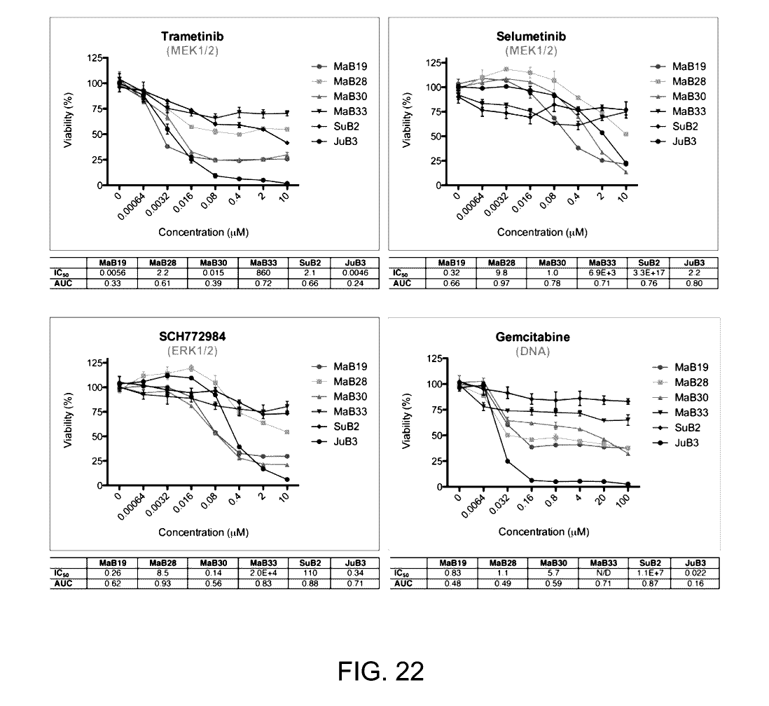

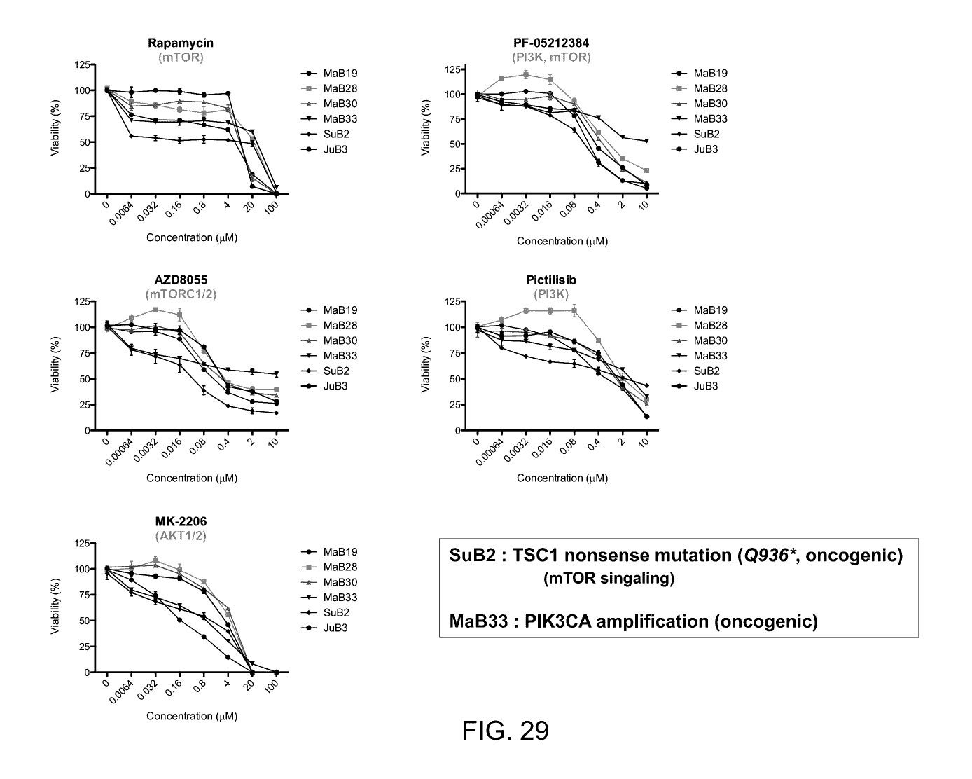

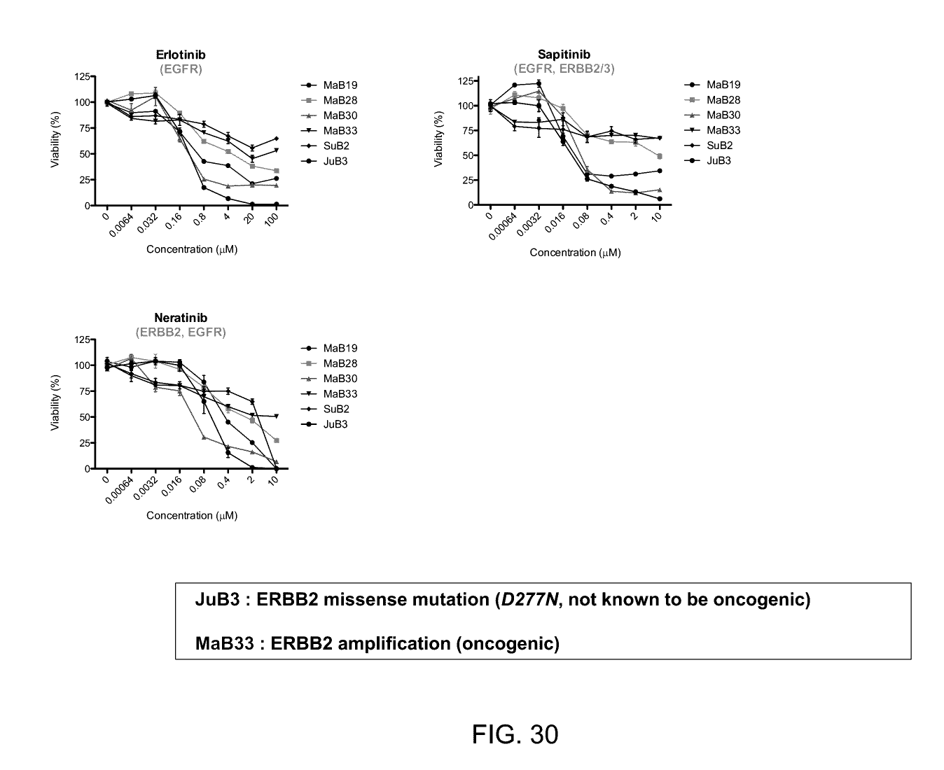

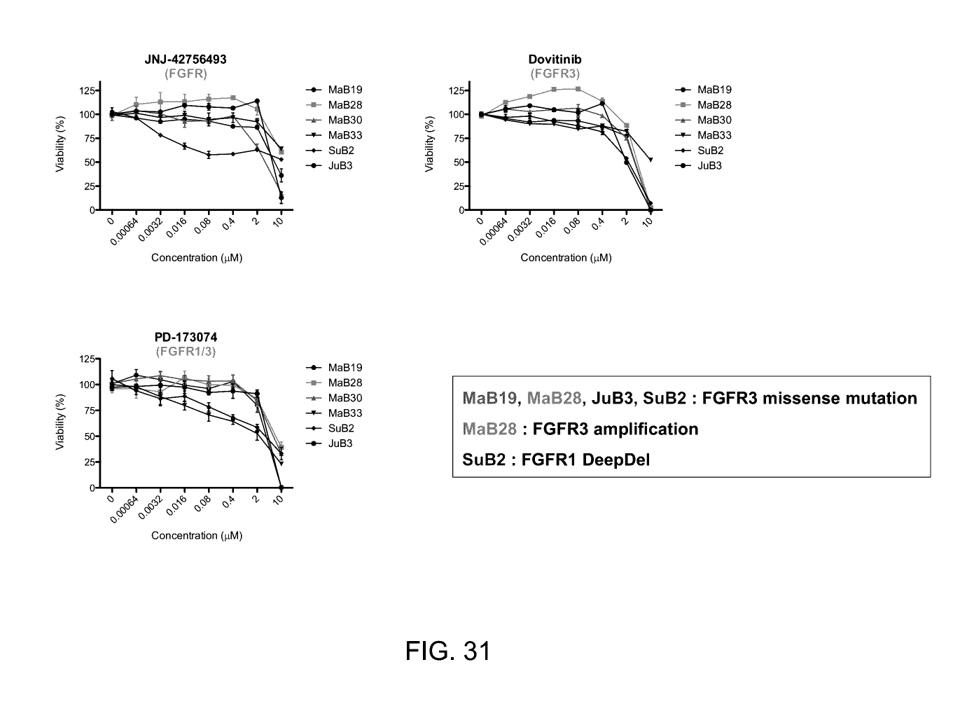

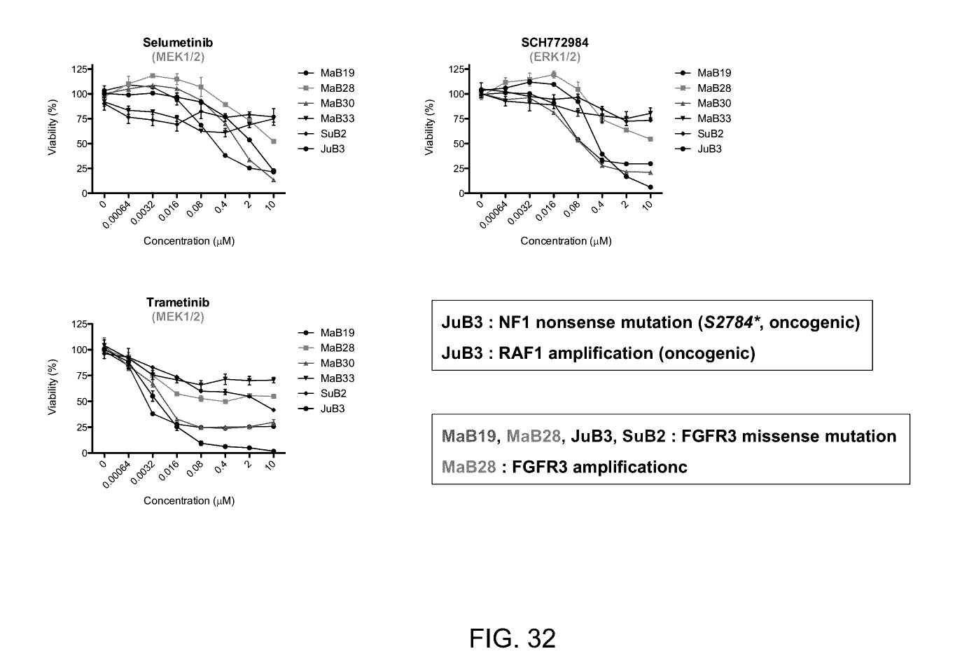

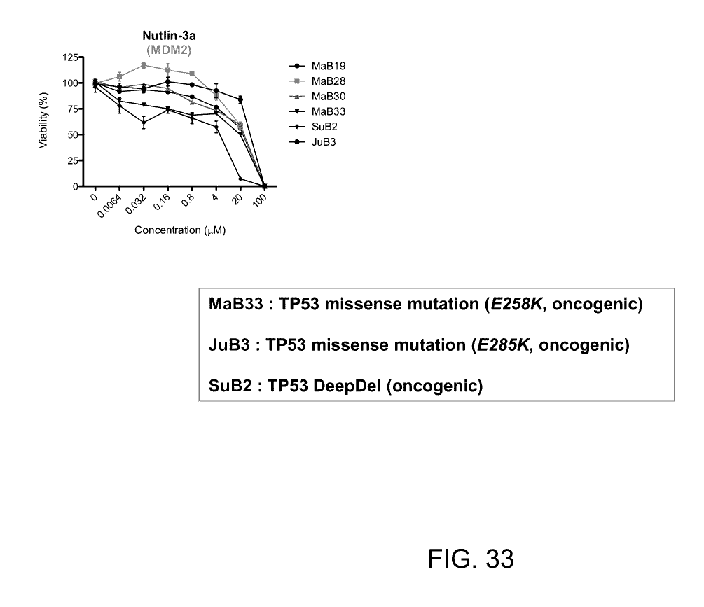

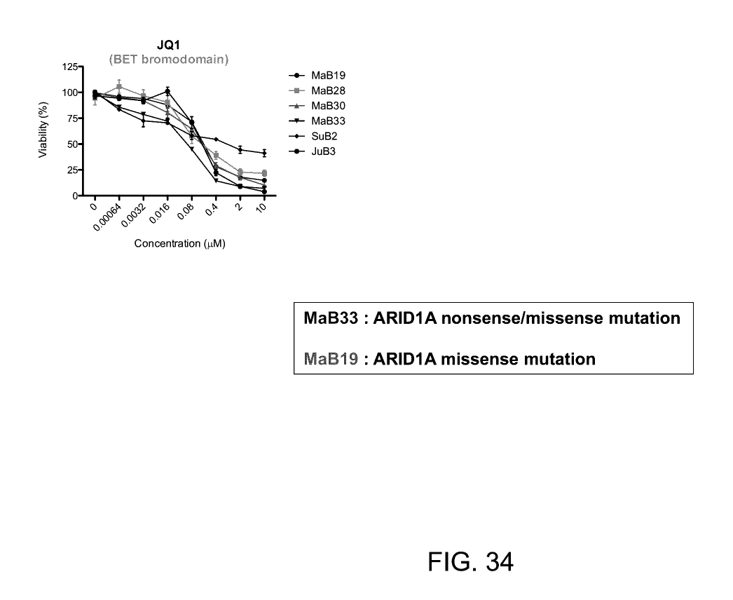

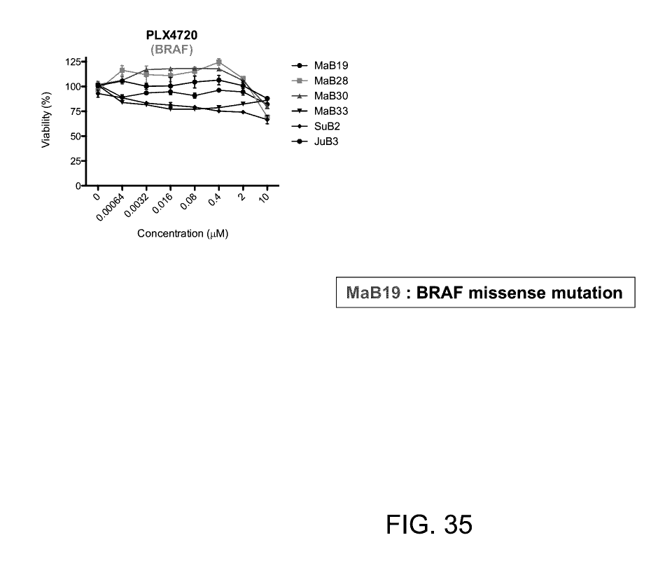

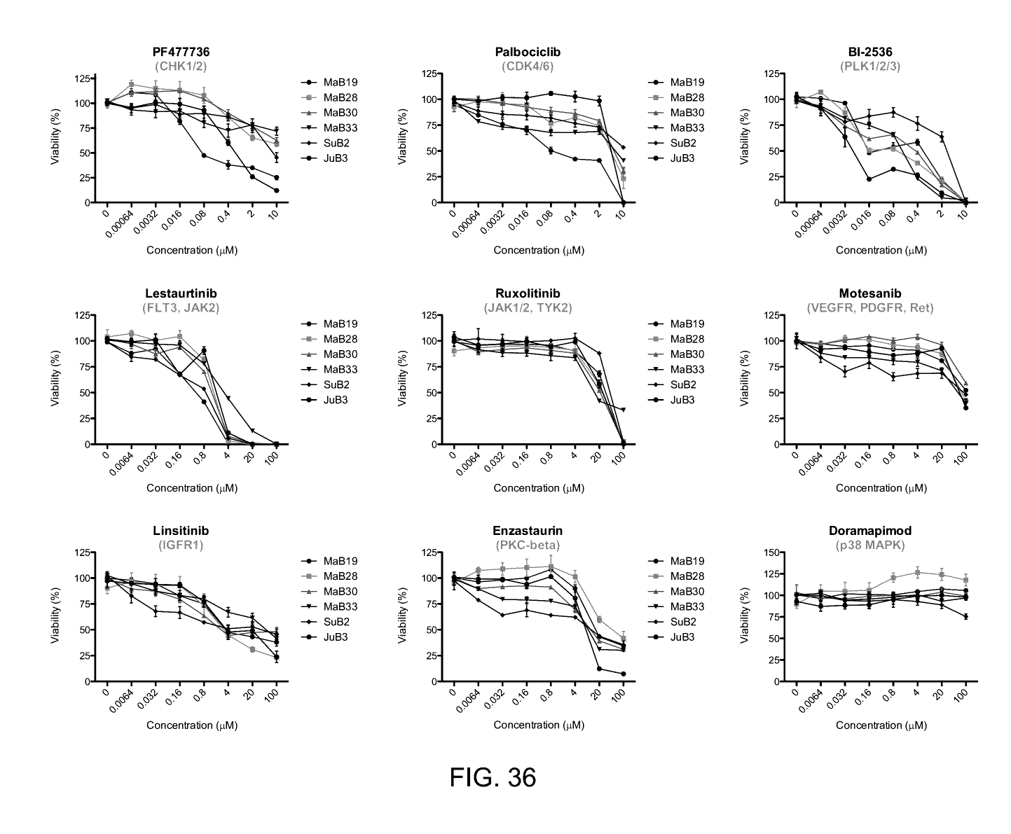

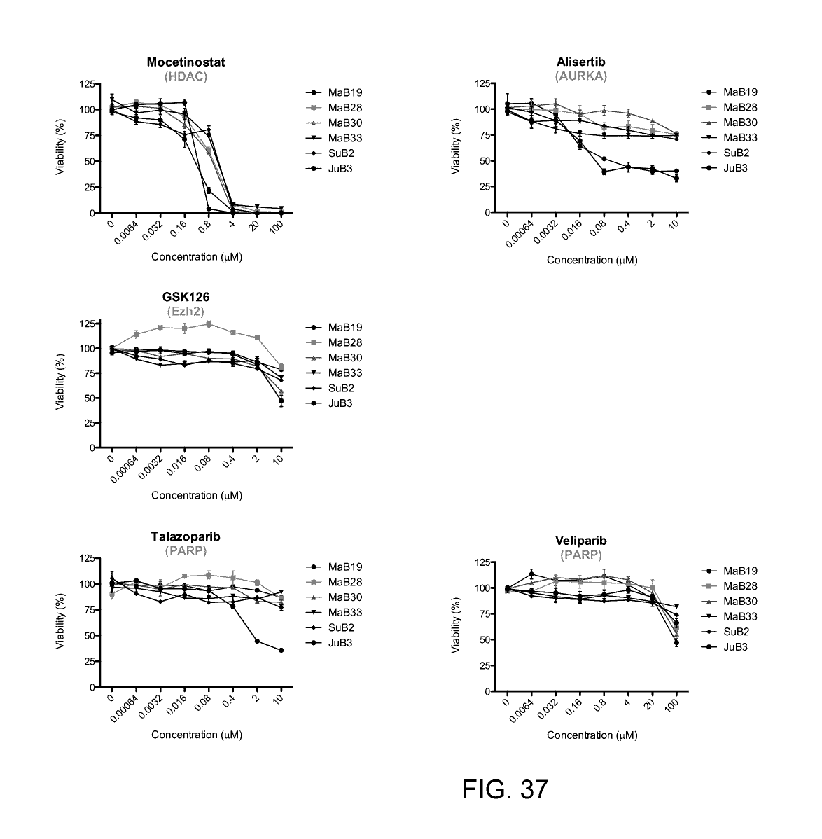

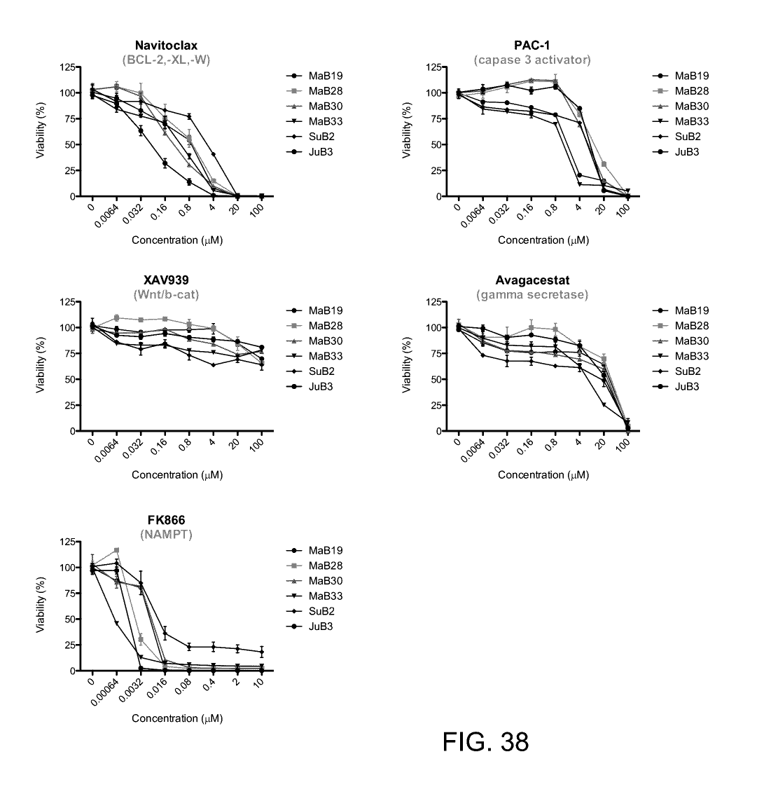

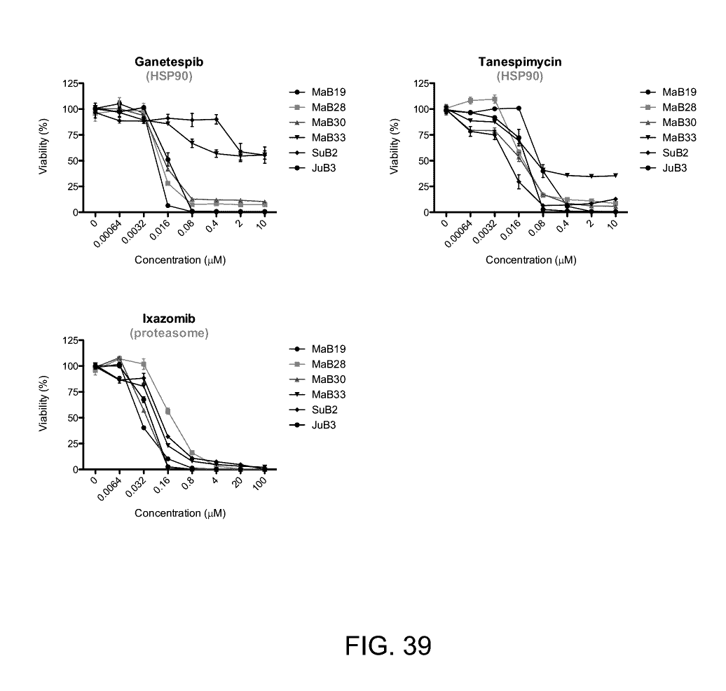

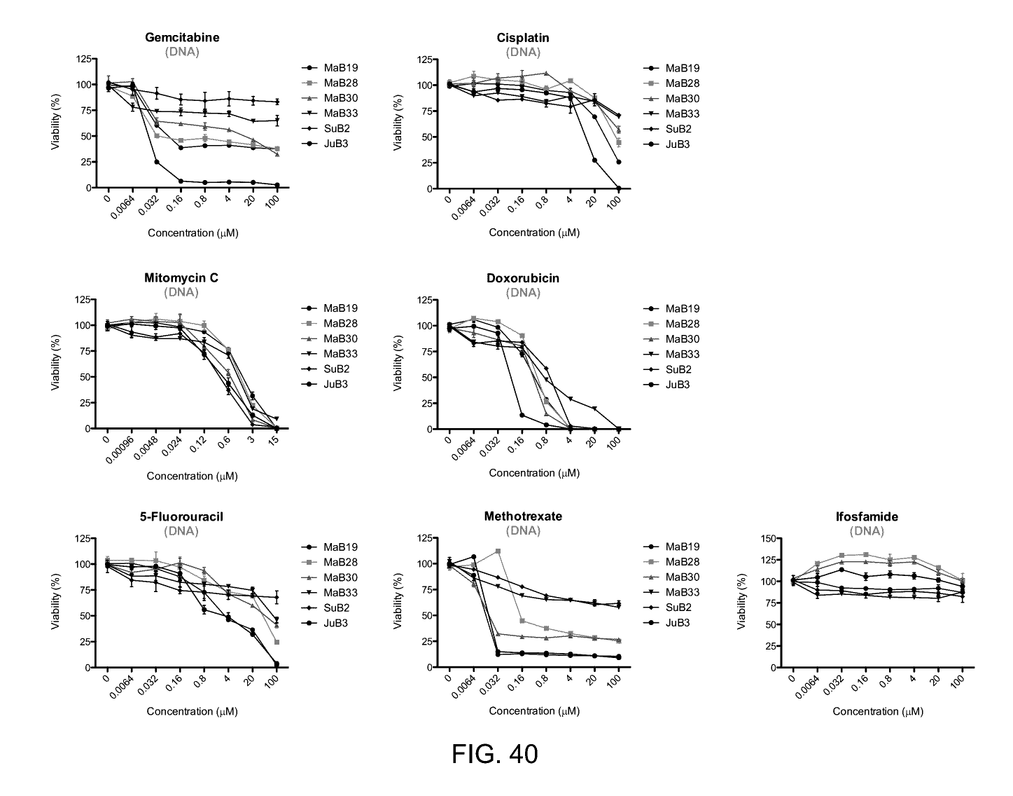

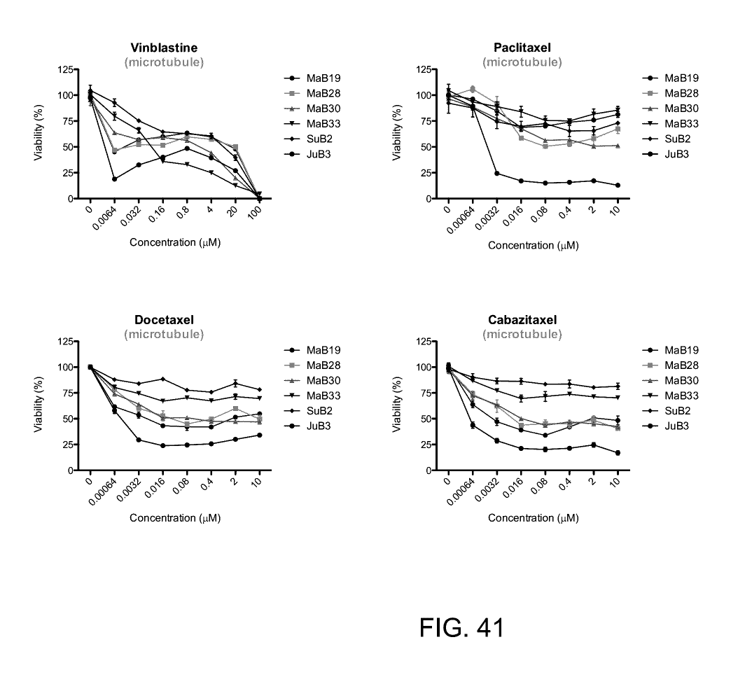

The response of patient-derived organoids and xenografts to inhibitors of tyrosine kinase receptor signaling pathways can be compared. To establish experimental conditions for these studies, a preliminary analysis of drug response in six independent patient-derived organoid lines has been performed. Dose titration assays were performed to examine the effects of treatment with four different compounds, corresponding to the small molecule MEK1/2 inhibitors trametinib and selumetinib, the ERK1/2 inhibitor SCH772984, and the nucleoside analog gemcitabine, which is a chemotherapy agent commonly used to treat patients with advanced bladder cancer (FIGS. 17-19; FIG. 22; see also FIGS. 29-41). Drug effects upon cell viability were assayed after treatment, and the resulting dose response curves were used to calculate values for IC₅₀ and area under the curve (AUC).

Striking differences were observed between the organoid lines in their sensitivity to these treatments. Notably, both MaB19 and JuB3 displayed significant responses to treatment with trametinib, selumetinib, and SCH772984, consistent with the presence of activating mutations in FGFR3. Conversely, however, MaB28 and SuB2 have FGFR3 mutations, but do not display a significant response to trametinib, selumetinib, and SCH772984, while the basis of the response of MaB30 to these agents is unclear. These preliminary findings demonstrate the feasibility of comparing the response of patient-derived organoids and xenografts to inhibitors, and indicate that mutational profiles can potentially explain some but not all of the differences in sensitivity and resistance of organoid cultures to clinical relevant compounds.

In summary, the data described herein have demonstrated the following key point. Patient-derived organoid lines as well as patient-derived xenografts have been successfully established. These lines recapitulate the histopathological phenotypes and mutational profiles of their corresponding parental tumors. Patient-derived organoids and xenografts can be interconverted with high efficiency, thereby generating matched pairs of organoid and xenograft lines. A sophisticated pipeline for the generation and analysis of targeted exome sequencing data for organoids and xenografts has been established. Patient-derived organoid lines can retain parental tumor heterogeneity, and at least some organoid lines display evidence of clonal evolution in culture. Drug response in organoids as well as xenografts can be readily assessed.

### Example 7: Research Design and Methods

Overview:

Based on the data described herein, matched patient-derived tumor organoid and xenograft lines can be used for comparative analyses of clonal evolution and drug response in human bladder cancer. The goal is to elucidate the relative advantages and disadvantages of these model systems in studies of bladder tumor biology, and to determine their accuracy in providing mechanistic insights into drug response. In particular, three aims will be pursued, as follows:

(1) To establish a biobank of patient-derived bladder tumor organoid and xenograft lines that is representative of the full spectrum of human bladder cancers, with a focus on the development of models from clinically aggressive variant subtypes that have a worse clinical prognosis, models that harbor potentially actionable genomic alterations, and models derived from tumors from women and underrepresented minorities;

(2) To pursue a comparative analysis of patient-derived organoid and xenograft lines to determine whether they capture the heterogeneity of the parental tumors, and undergo clonal evolution during serial passaging;

(3) To perform a comparative analysis of response to tyrosine kinase pathway inhibitors in bladder tumor organoids and xenografts to evaluate their potential for modeling patient responses.

Taken together, these findings should provide important reagents for the broader community of bladder cancer researchers, yield key insights into the advantages and disadvantages of organoid and xenograft models, and ultimately lead to the development of co-clinical trials to improve patient care.

As described in Example 6, an innovative methodology for three-dimensional culture of organoids obtained from fresh tissue biopsies of human bladder tumors from consented patients has been developed. To date, 15 independent organoid lines have been established from patient samples ranging from papillary non-invasive tumors to muscle-invasive cancer. These lines recapitulate the histopathological and molecular properties of their corresponding parental tumors, and targeted exome sequencing shows that they display genomic alterations characteristic of human bladder cancer. In parallel, a similar number of patient-derived xenograft lines have been established, and have shown that we can convert organoid lines into xenografts, and vice versa, thereby generating matched pairs of organoid and xenograft lines derived from the same parental tumors. Finally, it has been found that genomic alterations such as gain-of-function mutations of FGFR3 correlate at least in part with the response of organoid lines to drugs such as ERK (MAPK) pathway inhibitors.

Based on the results described in Example 6, these matched patient-derived tumor organoid and xenograft lines can be used for comparative analyses of clonal evolution and drug response in human bladder cancer. It will now be determined whether and how these model systems are most appropriate for studies of bladder tumor biology, and are most efficient and accurate in providing mechanistic insights into drug response. Our studies are highly innovative because they seek a precise delineation of the experimental advantages and disadvantages of organoid and xenograft approaches for investigation of patient-specific determinants of drug response, and thereby will provide the foundation for future development of effective co-clinical trials. Three specific aims can be pursued:

Establishment of a Biobank of Patient-Derived Bladder Tumor Organoid and Xenograft Lines.

The existing collection will be augmented by generating additional matched pairs from patients with rare bladder cancer subtypes and genomic alterations of interest, as well as from women and minorities. Histopathological and molecular analyses will be performed to assess the similarity of the organoids and xenografts to their corresponding parental tumors, and will use exome and RNA sequencing to categorize their genomic profiles and tumor subtype. Thus, a biobank of matched pairs of organoid and xenograft lines that is representative of the full spectrum of bladder cancer will be generated, and will ensure the authentication of this resource.

Comparative Analysis of Clonal Evolution in Patient-Derived Organoid and Xenograft Lines.

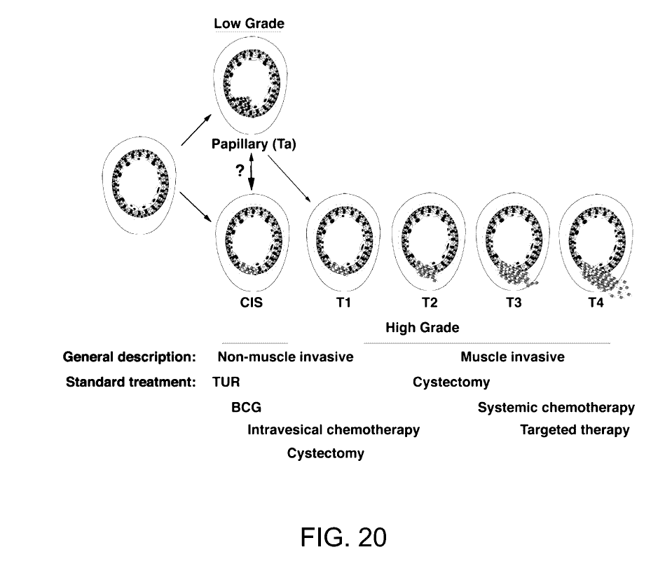

To determine whether tumor evolution can be accurately modeled in these systems, which is important for their relevance in studying treatment response, whether matched pairs of organoid and xenograft lines display parental tumor heterogeneity and clonal evolution during serial passaging will be examined. It will be determined whether the rates and outcomes of clonal evolution differ between organoid and xenografts, and whether expression of putative cancer stem cell markers correlates with changes in clonal populations. Xenograft tumors can be analyzed as described previously [41, 42, 88] and shown in FIG. 20.

Comparative Analysis of Response to Tyrosine Kinase Pathway Inhibitors in Bladder Tumor Organoids and Xenografts.

To assess their value for understanding treatment response in patients, the response of patient-derived organoid and xenograft lines to clinically-relevant compounds that target tyrosine kinase receptor pathways that are frequently activated in bladder cancer, including the FGFR3 and ERBB2 pathways will be compared. Potential correlations between the drug response of these lines with their corresponding phenotypes, genomic profiles, and potentially with the clinical response of the patient will be identified.

### REFERENCES FOR EXAMPLES 6 AND 7

1) Knowles, M. A. and Hurst, C. D. (2015). Molecular biology of bladder cancer: new insights into pathogenesis and clinical diversity. Nat Rev Cancer 15, 25-41.
2) Kamat, A. M., Hahn, N. M., Efstathiou, J. A., Lerner, S. P., Malmstrom, P. U., Choi, W., Guo, C. C., Lotan, Y. and Kassouf, W. (2016). Bladder cancer. Lancet 2016 Jun. 23, Epub ahead of print.
3) Kaufman, D. S., Shipley, W. U. and Feldman, A. S. (2009). Bladder cancer. Lancet 374, 239-249.
4) Prasad, S. M., Decastro, G. J., Steinberg, G. D. and Medscape. (2011). Urothelial carcinoma of the bladder: definition, treatment and future efforts. Nat Rev Urol 8, 631-642.
5) Botteman, M. F., Pashos, C. L., Redaelli, A., Laskin, B. and Hauser, R. (2003). The health economics of bladder cancer: a comprehensive review of the published literature. Pharmacoeconomics 21, 1315-1330.
6) Kobayashi, T., Owczarek, T. B., McKiernan, J. M. and Abate-Shen, C. (2015).
Modelling bladder cancer in mice: opportunities and challenges. Nat Rev Cancer 15, 42-54. PMCID: PMC4386904.
7) Hicks, R. M. (1975). The mammalian urinary bladder: an accommodating organ. Biol Rev Camb Philos Soc 50, 215-246.
8) Castillo-Martin, M., Domingo-Domenech, J., Karni-Schmidt, O., Matos, T. and Cordon-Cardo, C. (2010). Molecular pathways of urothelial development and bladder tumorigenesis. Urol Oncol 28, 401-408.
9) Gandhi, D., Molotkov, A., Batourina, E., Schneider, K., Dan, H., Reiley, M., Laufer, E., Metzger, D., Liang, F., Liao, Y., Sun, T. T., Aronow, B., Rosen, R., Mauney, J., Adam, R., Rosselot, C., Van Batavia, J., McMahon, A., McMahon, J., Guo, J. J. and Mendelsohn, C. (2013). Retinoid signaling in progenitors controls specification and regeneration of the urothelium. Dev Cell 26, 469-482. PMCID: PMC4024836.
10) Baskin, L. S., Hayward, S. W., Young, P. F. and Cunha, G. R. (1996). Ontogeny of the rat bladder: smooth muscle and epithelial differentiation. Acta Anat (Basel) 155, 163-171.
11) Jost, S. P. and Potten, C. S. (1986). Urothelial proliferation in growing mice. Cell Tissue Kinet 19, 155-160.
12) Cooper, E. H., Cowen, D. M. and Knowles, J. C. (1972). The recovery of mouse bladder epithelium after injury by 4-ethylsulphonylnaphthalene-1-sulphonamide. J Pathol 108, 151-156.
13) Ho, P. L., Kurtova, A. and Chan, K. S. (2012). Normal and neoplastic urothelial stem cells: getting to the root of the problem. Nat Rev Urol 9, 583-594. PMCID: PMC3468664.
14) Colopy, S. A., Bjorling, D. E., Mulligan, W. A. and Bushman, W. (2014). A population of progenitor cells in the basal and intermediate layers of the murine bladder urothelium contributes to urothelial development and regeneration. Dev Dyn 243, 988-998. PMCID: PMC4111772.
15) Papafotiou, G., Paraskevopoulou, V., Vasilaki, E., Kanaki, Z., Paschalidis, N. and Klinakis, A. (2016). KRT14 marks a subpopulation of bladder basal cells with pivotal role in regeneration and tumorigenesis. Nat Commun 7, 11914. PMCID: PMC4915139.
16) Shin, K., Lee, J., Guo, N., Kim, J., Lim, A., Qu, L., Mysorekar, I. U. and Beachy, P. A. (2011). Hedgehog/Wnt feedback supports regenerative proliferation of epithelial stem cells in bladder. Nature 472, 110-114. PMCID: PMC3676169.
17) Van Batavia, J., Yamany, T., Molotkov, A., Dan, H., Mansukhani, M., Batourina, E., Schneider, K., Oyon, D., Dunlop, M., Wu, X. R., Cordon-Cardo, C. and Mendelsohn, C. (2014). Bladder cancers arise from distinct urothelial sub-populations. Nat Cell Biol 16, 982-991, 981-985.
18) Shin, K., Lim, A., Odegaard, J. I., Honeycutt, J. D., Kawano, S., Hsieh, M. H. and Beachy, P. A. (2014). Cellular origin of bladder neoplasia and tissue dynamics of its progression to invasive carcinoma. Nat Cell Biol 16, 469-478. PMCID: PMC4196946.
19) Chan, K. S., Espinosa, I., Chao, M., Wong, D., Ailles, L., Diehn, M., Gill, H., Presti, J., Jr., Chang, H. Y., van de Rijn, M., Shortliffe, L. and Weissman, I. L. (2009). Identification, molecular characterization, clinical prognosis, and therapeutic targeting of human bladder tumor-initiating cells. Proc Natl Acad Sci USA 106, 14016-14021. PMCID: PMC2720852.
20) He, X., Marchionni, L., Hansel, D. E., Yu, W., Sood, A., Yang, J., Parmigiani, G., Matsui, W. and Berman, D. M. (2009). Differentiation of a highly tumorigenic basal cell compartment in urothelial carcinoma. Stem Cells 27, 1487-1495. PMCID: PMC3060766.
21) Volkmer, J. P., Sahoo, D., Chin, R. K., Ho, P. L., Tang, C., Kurtova, A. V., Willingham, S. B., Pazhanisamy, S. K., Contreras-Trujillo, H., Storm, T. A., Lotan, Y., Beck, A. H., Chung, B. I., Alizadeh, A. A., Godoy, G., Lerner, S. P., van de Rijn, M., Shortliffe, L. D., Weissman, I. L. and Chan, K. S. (2012). Three differentiation states risk-stratify bladder cancer into distinct subtypes. Proc Natl Acad Sci USA 109, 2078-2083. PMCID: PMC3277552.
22) Cancer Genome Atlas Research, N. (2014). Comprehensive molecular characterization of urothelial bladder carcinoma. Nature 507, 315-322. PMCID: PMC3962515.
23) Choi, W., Porten, S., Kim, S., Willis, D., Plimack, E. R., Hoffman-Censits, J., Roth, B., Cheng, T., Tran, M., Lee, I. L., Melquist, J., Bondaruk, J., Majewski, T., Zhang, S., Pretzsch, S., Baggerly, K., Siefker-Radtke, A., Czerniak, B., Dinney, C. P. and McConkey, D. J. (2014). Identification of distinct basal and luminal subtypes of muscle-invasive bladder cancer with different sensitivities to frontline chemotherapy. Cancer Cell 25, 152-165. PMCID: PMC4011497.
24) Damrauer, J. S., Hoadley, K. A., Chism, D. D., Fan, C., Tiganelli, C. J., Wobker, S. E., Yeh, J. J., Milowsky, M. I., Iyer, G., Parker, J. S. and Kim, W. Y. (2014). Intrinsic subtypes of high-grade bladder cancer reflect the hallmarks of breast cancer biology. Proc Natl Acad Sci USA 111, 3110-3115. PMCID: PMC3939870.
25) Dinney, C. P., McConkey, D. J., Millikan, R. E., Wu, X., Bar-Eli, M., Adam, L., Kamat, A. M., Siefker-Radtke, A. O., Tuziak, T., Sabichi, A. L., Grossman, H. B., Benedict, W. F. and Czerniak, B. (2004). Focus on bladder cancer. Cancer Cell 6, 111-116. PMID: 15324694.
26) Lerner, S. P., Bajorin, D. F., Dinney, C. P., Efstathiou, J. A., Groshen, S., Hahn, N. M., Hansel, D., Kwiatkowski, D., O'Donnell, M., Rosenberg, J., Svatek, R., Abrams, J. S., Al-Ahmadie, H., Apolo, A. B., Bellmunt, J., Callahan, M., Cha, E. K., Drake, C., Jarow, J., Kamat, A., Kim, W., Knowles, M., Mann, B., Marchionni, L., McConkey, D., McShane, L., Ramirez, N., Sharabi, A., Sharpe, A. H., Solit, D., Tangen, C. M., Amiri, A. T., Van Allen, E., West, P. J., Witjes, J. A. and Quale, D. Z. (2016). Summary and recommendations from the National Cancer Institute's clinical trials planning meeting on novel therapeutics for non-muscle invasive bladder cancer. Bladder Cancer 2, 165-202. PMCID: PMC4927845.
27) Wu, X., Hildebrandt, M. A. and Chang, D. W. (2009). Genome-wide association studies of bladder cancer risk: a field synopsis of progress and potential applications. Cancer Metastasis Rev 28, 269-280.
28) Freedman, N. D., Silverman, D. T., Hollenbeck, A. R., Schatzkin, A. and Abnet, C. C. (2011). Association between smoking and risk of bladder cancer among men and women. JAMA 306, 737-745. PMCID: PMC3441175.
29) Kiriluk, K. J., Prasad, S. M., Patel, A. R., Steinberg, G. D. and Smith, N. D. (2012). Bladder cancer risk from occupational and environmental exposures. Urol Oncol 30, 199-211.
30) Crivelli, J. J., Xylinas, E., Kluth, L. A., Rieken, M., Rink, M. and Shariat, S. F. (2014). Effect of smoking on outcomes of urothelial carcinoma: a systematic review of the literature. Eur Urol 65, 742-754.
31) Burger, M., Catto, J. W., Dalbagni, G., Grossman, H. B., Herr, H., Karakiewicz, P., Kassouf, W., Kiemeney, L. A., La Vecchia, C., Shariat, S. and Lotan, Y. (2013). Epidemiology and risk factors of urothelial bladder cancer. Eur Urol 63, 234-241.
32) Lucca, I., Klatte, T., Fajkovic, H., de Martino, M. and Shariat, S. F. (2015). Gender differences in incidence and outcomes of urothelial and kidney cancer. Nat Rev Urol 12, 585-592.
33) Lucca, I., Fajkovic, H. and Klatte, T. (2014). Sex steroids and gender differences in nonmuscle invasive bladder cancer. Curr Opin Urol 24, 500-505.
34) Hsu, J. W., Hsu, I., Xu, D., Miyamoto, H., Liang, L., Wu, X. R., Shyr, C. R. and Chang, C. (2013). Decreased tumorigenesis and mortality from bladder cancer in mice lacking urothelial androgen receptor. Am J Pathol 182, 1811-1820. PMCID: PMC3644728.
35) Lin, C., Yin, Y., Stemler, K., Humphrey, P., Kibel, A. S., Mysorekar, I. U. and Ma, L. (2013). Constitutive beta-catenin activation induces male-specific tumorigenesis in the bladder urothelium. Cancer Res 73, 5914-5925. PMCID: PMC3790859.
36) Resnick, M. J., Bassett, J. C. and Clark, P. E. (2013). Management of superficial and muscle-invasive urothelial cancers of the bladder. Curr Opin Oncol 25, 281-288.
37) Clark, P. E., Agarwal, N., Biagioli, M. C., Eisenberger, M. A., Greenberg, R. E., Herr, H. W., Inman, B. A., Kuban, D. A., Kuzel, T. M., Lele, S. M., Michalski, J., Pagliaro, L. C., Pal, S. K., Patterson, A., Plimack, E. R., Pohar, K. S., Porter, M. P., Richie, J. P., Sexton, W. J., Shipley, W. U., Small, E. J., Spiess, P. E., Trump, D. L., Wile, G., Wilson, T. G., Dwyer, M., Ho, M. and National Comprehensive Cancer, N. (2013). Bladder cancer. J Natl Compr Canc Netw 11, 446-475.
38) Redelman-Sidi, G., Glickman, M. S. and Bochner, B. H. (2014). The mechanism of action of BCG therapy for bladder cancer—a current perspective. Nat Rev Urol 11, 153-162.
39) Herr, H. W. and Morales, A. (2008). History of bacillus Calmette-Guerin and bladder cancer: an immunotherapy success story. J Urol 179, 53-56.
40) Barlow, L. J., Seager, C. M., Benson, M. C. and McKiernan, J. M. (2010). Novel intravesical therapies for non-muscle-invasive bladder cancer refractory to BCG. Urol Oncol 28, 108-111.
41) Delto, J. C., Kobayashi, T., Benson, M., McKiernan, J. and Abate-Shen, C. (2013). Preclinical analyses of intravesical chemotherapy for prevention of bladder cancer progression. Oncotarget 4, 269-276. PMCID: PMC3712572.
42) Seager, C. M., Puzio-Kuter, A. M., Patel, T., Jain, S., Cordon-Cardo, C., Mc Kiernan, J. and Abate-Shen, C. (2009). Intravesical delivery of rapamycin suppresses tumorigenesis in a mouse model of progressive bladder cancer. Cancer Prev Res (Phila) 2, 1008-1014. PMCID: PMC2789170.
43) Zargar, H., Espiritu, P. N., Fairey, A. S., Mertens, L. S., Dinney, C. P., Mir, M. C., Krabbe, L. M., Cookson, M. S., Jacobsen, N. E., Gandhi, N. M., Griffin, J., Montgomery, J. S., Vasdev, N., Yu, E. Y., Youssef, D., Xylinas, E., Campain, N. J., Kassouf, W., Dall′Era, M. A., Seah, J. A., Ercole, C. E., Horenblas, S., Sridhar, S. S., McGrath, J. S., Aning, J., Shariat, S. F., Wright, J. L., Thorpe, A. C., Morgan, T. M., Holzbeierlein, J. M., Bivalacqua, T. J., North, S., Barocas, D. A., Lotan, Y., Garcia, J. A., Stephenson, A. J., Shah, J. B., van Rhijn, B. W., Daneshmand, S., Spiess, P. E. and Black, P. C. (2015). Multicenter assessment of neoadjuvant chemotherapy for muscle-invasive bladder cancer. Eur Urol 67, 241-249. PMCID: PMC4840190.
44) Dash, A., Pettus, J. A. t., Herr, H. W., Bochner, B. H., Dalbagni, G., Donat, S. M., Russo, P., Boyle, M. G., Milowsky, M. I. and Bajorin, D. F. (2008). A role for neoadjuvant gemcitabine plus cisplatin in muscle-invasive urothelial carcinoma of the bladder: a retrospective experience. Cancer 113, 2471-2477. PMCID: PMC2585515.
45) Bochner, B. H. (2011). Chemotherapy: Standardizing the care of invasive bladder cancer. Nat Rev Clin Oncol 8, 454-455.
46) Powles, T., Eder, J. P., Fine, G. D., Braiteh, F. S., Loriot, Y., Cruz, C., Bellmunt, J., Burris, H. A., Petrylak, D. P., Teng, S. L., Shen, X., Boyd, Z., Hegde, P. S., Chen, D. S. and Vogelzang, N. J. (2014). MPDL3280A (anti-PD-L1) treatment leads to clinical activity in metastatic bladder cancer. Nature 515, 558-562.
47) Rosenberg, J. E., Hoffman-Censits, J., Powles, T., van der Heijden, M. S., Baler, A. V., Necchi, A., Dawson, N., O'Donnell, P. H., Balmanoukian, A., Loriot, Y., Srinivas, S., Retz, M. M., Grivas, P., Joseph, R. W., Galsky, M. D., Fleming, M. T., Petrylak, D. P., Perez-Gracia, J. L., Burris, H. A., Castellano, D., Canil, C., Bellmunt, J., Bajorin, D., Nickles, D., Bourgon, R., Frampton, G. M., Cui, N., Mariathasan, S., Abidoye, O., Fine, G. D. and Dreicer, R. (2016). Atezolizumab in patients with locally advanced and metastatic urothelial carcinoma who have progressed following treatment with platinum-based chemotherapy: a single-arm, multicentre, phase 2 trial. Lancet 387, 1909-1920.
48) Dyrskjot, L., Thykjaer, T., Kruhoffer, M., Jensen, J. L., Marcussen, N., Hamilton-Dutoit, S., Wolf, H. and Orntoft, T. F. (2003). Identifying distinct classes of bladder carcinoma using microarrays. Nat Genet 33, 90-96.
49) Kim, J. H., Tuziak, T., Hu, L., Wang, Z., Bondaruk, J., Kim, M., Fuller, G., Dinney, C., Grossman, H. B., Baggerly, K., Zhang, W. and Czerniak, B. (2005). Alterations in transcription clusters underlie development of bladder cancer along papillary and nonpapillary pathways. Lab Invest 85, 532-549. PMID: 15778693.
50) Lindgren, D., Frigyesi, A., Gudjonsson, S., Sjodahl, G., Hallden, C., Chebil, G., Veerla, S., Ryden, T., Mansson, W., Liedberg, F. and Hoglund, M. (2010). Combined gene expression and genomic profiling define two intrinsic molecular subtypes of urothelial carcinoma and gene signatures for molecular grading and outcome. Cancer Res 70, 3463-3472. PMID: 20406976.
51) Blaveri, E., Brewer, J. L., Roydasgupta, R., Fridlyand, J., DeVries, S., Koppie, T., Pejavar, S., Mehta, K., Carroll, P., Simko, J. P. and Waldman, F. M. (2005). Bladder cancer stage and outcome by array-based comparative genomic hybridization. Clin Cancer Res 11, 7012-7022. PMID: 16203795.
52) Lauss, M., Ringner, M. and Hoglund, M. (2010). Prediction of stage, grade, and survival in bladder cancer using genome-wide expression data: a validation study. Clin Cancer Res 16, 4421-4433. PMID: 20736328.
53) Zieger, K., Marcussen, N., Borre, M., Orntoft, T. F. and Dyrskjot, L. (2009). Consistent genomic alterations in carcinoma in situ of the urinary bladder confirm the presence of two major pathways in bladder cancer development. Int J Cancer 125, 2095-2103. PMID: 19637316.
54) Hurst, C. D., Platt, F. M., Taylor, C. F. and Knowles, M. A. (2012). Novel tumor subgroups of urothelial carcinoma of the bladder defined by integrated genomic analysis. Clin Cancer Res 18, 5865-5877. PMID: 22932667.
55) Lindgren, D., Sjodahl, G., Lauss, M., Staaf, J., Chebil, G., Lovgren, K., Gudjonsson, S., Liedberg, F., Patschan, O., Mansson, W., Ferno, M. and Hoglund, M. (2012). Integrated genomic and gene expression profiling identifies two major genomic circuits in urothelial carcinoma. PLoS One 7, e38863. PMCID: PMC3369837.
56) Hedegaard, J., Lamy, P., Nordentoft, I., Algaba, F., Hoyer, S., Ulhoi, B. P., Vang, S. Reinert, T., Hermann, G. G., Mogensen, K., Thomsen, M. B., Nielsen, M. M. Marquez, M., Segersten, U., Aine, M., Hoglund, M., Birkenkamp-Demtroder, K. Fristrup, N., Borre, M., Hartmann, A., Stohr, R., Wach, S., Keck, B., Seitz, A. K. Nawroth, R., Maurer, T., Tulic, C., Simic, T., Junker, K., Horstmann, M., Harving, N. Petersen, A. C., Calle, M. L., Steyerberg, E. W., Beukers, W., van Kessel, K. E., Jensen, J. B., Pedersen, J. S., Malmstrom, P. U., Malats, N., Real, F. X., Zwarthoff, E. C., Orntoft, T. F. and Dyrskjot, L. (2016). Comprehensive Transcriptional Analysis of Early-Stage Urothelial Carcinoma. Cancer Cell 30, 27-42.
57) Gui, Y., Guo, G., Huang, Y., Hu, X., Tang, A., Gao, S., Wu, R., Chen, C., Li, X., Zhou, L., He, M., Li, Z., Sun, X., Jia, W., Chen, J., Yang, S., Zhou, F., Zhao, X., Wan, S., Ye, R., Liang, C., Liu, Z., Huang, P., Liu, C., Jiang, H., Wang, Y., Zheng, H., Sun, L., Liu, X., Jiang, Z., Feng, D., Chen, J., Wu, S., Zou, J., Zhang, Z., Yang, R., Zhao, J., Xu, C., Yin, W., Guan, Z., Ye, J., Zhang, H., Li, J., Kristiansen, K., Nickerson, M. L., Theodorescu, D., Li, Y., Zhang, X., Li, S., Wang, J., Yang, H., Wang, J. and Cai, Z. (2011). Frequent mutations of chromatin remodeling genes in transitional cell carcinoma of the bladder. Nat Genet 43, 875-878.
58) Guo, G., Sun, X., Chen, C., Wu, S., Huang, P., Li, Z., Dean, M., Huang, Y., Jia, W., Zhou, Q., Tang, A., Yang, Z., Li, X., Song, P., Zhao, X., Ye, R., Zhang, S., Lin, Z., Qi, M., Wan, S., Xie, L., Fan, F., Nickerson, M. L., Zou, X., Hu, X., Xing, L., Lv, Z., Mei, H., Gao, S., Liang, C., Gao, Z., Lu, J., Yu, Y., Liu, C., Li, L., Fang, X., Jiang, Z., Yang, J., Li, C., Zhao, X., Chen, J., Zhang, F., Lai, Y., Lin, Z., Zhou, F., Chen, H., Chan, H. C., Tsang, S., Theodorescu, D., Li, Y., Zhang, X., Wang, J., Yang, H., Gui, Y., Wang, J. and Cai, Z. (2013). Whole-genome and whole-exome sequencing of bladder cancer identifies frequent alterations in genes involved in sister chromatid cohesion and segregation. Nat Genet 45, 1459-1463.
59) Morrison, C. D., Liu, P., Woloszynska-Read, A., Zhang, J., Luo, W., Qin, M., Bshara, W., Conroy, J. M., Sabatini, L., Vedell, P., Xiong, D., Liu, S., Wang, J., Shen, H., Li, Y., Omilian, A. R., Hill, A., Head, K., Guru, K., Kunnev, D., Leach, R., Eng, K. H., Darlak, C., Hoeflich, C., Veeranki, S., Glenn, S., You, M., Pruitt, S. C., Johnson, C. S. and Trump, D. L. (2014). Whole-genome sequencing identifies genomic heterogeneity at a nucleotide and chromosomal level in bladder cancer. Proc Natl Acad Sci USA 111, E672-681. PMCID: PMC3926024.
60) Cazier, J. B., Rao, S. R., McLean, C. M., Walker, A. L., Wright, B. J., Jaeger, E. E., Kartsonaki, C., Marsden, L., Yau, C., Camps, C., Kaisaki, P., Oxford-Illumina, W. G. S. C., Taylor, J., Catto, J. W., Tomlinson, I. P., Kiltie, A. E. and Hamdy, F. C. (2014). Whole-genome sequencing of bladder cancers reveals somatic CDKN1A mutations and clinicopathological associations with mutation burden. Nat Commun 5, 3756. PMCID: PMC4010643.
61) Al-Ahmadie, H. A., lyer, G., Janakiraman, M., Lin, O., Heguy, A., Tickoo, S. K., Fine, S. W., Gopalan, A., Chen, Y. B., Balar, A., Riches, J., Bochner, B., Dalbagni, G., Bajorin, D. F., Reuter, V. E., Milowsky, M. I. and Solit, D. B. (2011). Somatic mutation of fibroblast growth factor receptor-3 (FGFR3) defines a distinct morphological subtype of high-grade urothelial carcinoma. J Pathol 224, 270-279. PMCID: PMC3235805.
62) lyer, G., Al-Ahmadie, H., Schultz, N., Hanrahan, A. J., Ostrovnaya, I., Balar, A. V., Kim, P. H., Lin, O., Weinhold, N., Sander, C., Zabor, E. C., Janakiraman, M., Garcia-Grossman, I. R., Heguy, A., Viale, A., Bochner, B. H., Reuter, V. E., Bajorin, D. F., Milowsky, M. I., Taylor, B. S. and Solit, D. B. (2013). Prevalence and co-occurrence of actionable genomic alterations in high-grade bladder cancer. J Clin Oncol 31, 3133-3140. PMCID: PMC3753703.
63) lyer, G., Hanrahan, A. J., Milowsky, M. I., Al-Ahmadie, H., Scott, S. N., Janakiraman, M., Pirun, M., Sander, C., Socci, N. D., Ostrovnaya, I., Viale, A., Heguy, A., Peng, L., Chan, T. A., Bochner, B., Bajorin, D. F., Berger, M. F., Taylor, B. S. and Solit, D. B. (2012). Genome sequencing identifies a basis for everolimus sensitivity. Science 338, 221. PMCID: PMC3633467.
64) Wagle, N., Grabiner, B. C., Van Allen, E. M., Hodis, E., Jacobus, S., Supko, J. G., Stewart, M., Choueiri, T. K., Gandhi, L., Cleary, J. M., Elfiky, A. A., Taplin, M. E., Stack, E. C., Signoretti, S., Loda, M., Shapiro, G. I., Sabatini, D. M., Lander, E. S., Gabriel, S. B., Kantoff, P. W., Garraway, L. A. and Rosenberg, J. E. (2014). Activating mTOR mutations in a patient with an extraordinary response on a phase I trial of everolimus and pazopanib. Cancer Discov 4, 546-553. PMCID: PMC4122326.
65) Shih, C. and Weinberg, R. A. (1982). Isolation of a transforming sequence from a human bladder carcinoma cell line. Cell 29, 161-169.
66) Reddy, E. P., Reynolds, R. K., Santos, E. and Barbacid, M. (1982). A point mutation is responsible for the acquisition of transforming properties by the T24 human bladder carcinoma oncogene. Nature 300, 149-152.
67) Gust, K. M., McConkey, D. J., Awrey, S., Hegarty, P. K., Qing, J., Bondaruk, J., Ashkenazi, A., Czerniak, B., Dinney, C. P. and Black, P. C. (2013). Fibroblast growth factor receptor 3 is a rational therapeutic target in bladder cancer. Mol Cancer Ther 12, 1245-1254. PMCID: PMC3707970.
68) Rebouissou, S., Bernard-Pierrot, I., de Reynies, A., Lepage, M. L., Krucker, C., Chapeaublanc, E., Herault, A., Kamoun, A., Caillault, A., Letouze, E., Elarouci, N., Neuzillet, Y., Denoux, Y., Molinie, V., Vordos, D., Laplanche, A., Maille, P., Soyeux, P., Ofualuka, K., Reyal, F., Biton, A., Sibony, M., Paoletti, X., Southgate, J., Benhamou, S., Lebret, T., Allory, Y. and Radvanyi, F. (2014). EGFR as a potential therapeutic target for a subset of muscle-invasive bladder cancers presenting a basal-like phenotype. Sci Transl Med 6, 244ra291. PMID: 25009231.
69) Lee, J. K., Havaleshko, D. M., Cho, H., Weinstein, J. N., Kaldjian, E. P., Karpovich, J., Grimshaw, A. and Theodorescu, D. (2007). A strategy for predicting the chemosensitivity of human cancers and its application to drug discovery. Proc Natl Acad Sci USA 104, 13086-13091. PMCID: PMC1941805.
70) Havaleshko, D. M., Cho, H., Conaway, M., Owens, C. R., Hampton, G., Lee, J. K. and Theodorescu, D. (2007). Prediction of drug combination chemosensitivity in human bladder cancer. Mol Cancer Ther 6, 578-586. PMID: 17308055.
71) Borah, S., Xi, L., Zaug, A. J., Powell, N. M., Dancik, G. M., Cohen, S. B., Costello, J. C., Theodorescu, D. and Cech, T. R. (2015). Cancer. TERT promoter mutations and telomerase reactivation in urothelial cancer. Science 347, 1006-1010. PMCID: PMC4640672.
72) Al-Ahmadie, H. A., Iyer, G., Lee, B. H., Scott, S. N., Mehra, R., Bagrodia, A., Jordan, E. J., Gao, S. P., Ramirez, R., Cha, E. K., Desai, N. B., Zabor, E. C., Ostrovnaya, I., Gopalan, A., Chen, Y. B., Fine, S. W., Tickoo, S. K., Gandhi, A., Hreiki, J., Viale, A., Arcila, M. E., Dalbagni, G., Rosenberg, J. E., Bochner, B. H., Bajorin, D. F., Berger, M. F., Reuter, V. E., Taylor, B. S. and Solit, D. B. (2016). Frequent somatic CDH1 loss-of-function mutations in plasmacytoid variant bladder cancer. Nat Genet 48, 356-358. PMCID: PMC4827439.
73) DeGraff, D. J., Robinson, V. L., Shah, J. B., Brandt, W. D., Sonpavde, G., Kang, Y., Liebert, M., Wu, X. R., Taylor, J. A., 3rd and Translational Science Working Group of the Bladder Advocacy Network Think, T. (2013). Current preclinical models for the advancement of translational bladder cancer research. Mol Cancer Ther 12, 121-130. PMID: 23269072.
74) Barretina, J., Caponigro, G., Stransky, N., Venkatesan, K., Margolin, A. A., Kim, S., Wilson, C. J., Lehar, J., Kryukov, G. V., Sonkin, D., Reddy, A., Liu, M., Murray, L., Berger, M. F., Monahan, J. E., Morais, P., Meltzer, J., Korejwa, A., Jane-Valbuena, J., Mapa, F. A., Thibault, J., Bric-Furlong, E., Raman, P., Shipway, A., Engels, I. H., Cheng, J., Yu, G. K., Yu, J., Aspesi, P., Jr., de Silva, M., Jagtap, K., Jones, M. D., Wang, L., Hatton, C., Palescandolo, E., Gupta, S., Mahan, S., Sougnez, C., Onofrio, R. C., Liefeld, T., MacConaill, L., Winckler, W., Reich, M., Li, N., Mesirov, J. P., Gabriel, S. B., Getz, G., Ardlie, K., Chan, V., Myer, V. E., Weber, B. L., Porter, J., Warmuth, M., Finan, P., Harris, J. L., Meyerson, M., Golub, T. R., Morrissey, M. P., Sellers, W. R., Schlegel, R. and Garraway, L. A. (2012). The Cancer Cell Line Encyclopedia enables predictive modelling of anticancer drug sensitivity. Nature 483, 603-607. PMCID: PMC3320027.
75) Tentler, J. J., Tan, A. C., Weekes, C. D., Jimeno, A., Leong, S., Pitts, T. M., Arcaroli, J. J., Messersmith, W. A. and Eckhardt, S. G. (2012). Patient-derived tumour xenografts as models for oncology drug development. Nat Rev Clin Oncol 9, 338-350. PMCID: PMC3928688.
76) Hidalgo, M., Amant, F., Biankin, A. V., Budinska, E., Byrne, A. T., Caldas, C., Clarke, R. B., de Jong, S., Jonkers, J., Maelandsmo, G. M., Roman-Roman, S., Seoane, J., Trusolino, L. and Villanueva, A. (2014). Patient-derived xenograft models: an emerging platform for translational cancer research. Cancer Discov 4, 998-1013. PMCID: PMC4167608.
77) Park, B., Jeong, B. C., Choi, Y. L., Kwon, G. Y., Lim, J. E., Seo, S. I., Jeon, S. S., Lee, H. M., Choi, H. Y. and Lee, K. S. (2013). Development and characterization of a bladder cancer xenograft model using patient-derived tumor tissue. Cancer Sci 104, 631-638. PMID: 23384396.
78) Jager, W., Xue, H., Hayashi, T., Janssen, C., Awrey, S., Wyatt, A. W., Anderson, S., Moskalev, I., Haegert, A., Alshalalfa, M., Erho, N., Davicioni, E., Fazli, L., Li, E., Collins, C., Wang, Y. and Black, P. C. (2015). Patient-derived bladder cancer xenografts in the preclinical development of novel targeted therapies. Oncotarget 6, 21522-21532. PMCID: PMC4673283.
79) Cirone, P., Andresen, C. J., Eswaraka, J. R., Lappin, P. B. and Bagi, C. M. (2014). Patient-derived xenografts reveal limits to PI3K/mTOR- and MEK-mediated inhibition of bladder cancer. Cancer Chemother Pharmacol 73, 525-538.
80) Boj, S. F., Hwang, C. I., Baker, L. A., Chio, II, Engle, D. D., Corbo, V., Jager, M., Ponz-Sarvise, M., Tiriac, H., Spector, M. S., Gracanin, A., Oni, T., Yu, K. H., van Boxtel, R., Huch, M., Rivera, K. D., Wilson, J. P., Feigin, M. E., Ohlund, D., Handly-Santana, A., Ardito-Abraham, C. M., Ludwig, M., Elyada, E., Alagesan, B., Biffi, G., Yordanov, G. N., Delcuze, B., Creighton, B., Wright, K., Park, Y., Morsink, F. H., Molenaar, I. Q., Borel Rinkes, I. H., Cuppen, E., Hao, Y., Jin, Y., Nijman, I. J., Iacobuzio-Donahue, C., Leach, S. D., Pappin, D. J., Hammell, M., Klimstra, D. S., Basturk, O., Hruban, R. H., Offerhaus, G. J., Vries, R. G., Clevers, H. and Tuveson, D. A. (2015). Organoid models of human and mouse ductal pancreatic cancer. Cell 160, 324-338. PMCID: PMC4334572.
81) Gao, D., Vela, I., Sboner, A., laquinta, P. J., Karthaus, W. R., Gopalan, A., Dowling, C., Wanjala, J. N., Undvall, E. A., Arora, V. K., Wongvipat, J., Kossai, M., Ramazanoglu, S., Barboza, L. P., Di, W., Cao, Z., Zhang, Q. F., Sirota, I., Ran, L., MacDonald, T. Y., Beltran, H., Mosquera, J. M., Touijer, K. A., Scardino, P. T., Laudone, V. P., Curtis, K. R., Rathkopf, D. E., Morris, M. J., Danila, D. C., Slovin, S. F., Solomon, S. B., Eastham, J. A., Chi, P., Carver, B., Rubin, M. A., Scher, H. I., Clevers, H., Sawyers, C. L. and Chen, Y. (2014). Organoid cultures derived from patients with advanced prostate cancer. Cell 159, 176-187. PMCID: PMC4237931.
82) van de Wetering, M., Francies, H. E., Francis, J. M., Bounova, G., lorio, F., Pronk, A., van Houdt, W., van Gorp, J., Taylor-Weiner, A., Kester, L., McLaren-Douglas, A., Blokker, J., Jaksani, S., Bartfeld, S., Volckman, R., van Sluis, P., Li, V. S., Seepo, S., Sekhar Pedamallu, C., Cibulskis, K., Carter, S. L., McKenna, A., Lawrence, M. S., Lichtenstein, L., Stewart, C., Koster, J., Versteeg, R., van Oudenaarden, A., Saez-Rodriguez, J., Vries, R. G., Getz, G., Wessels, L., Stratton, M. R., McDermott, U., Meyerson, M., Garnett, M. J. and Clevers, H. (2015). Prospective derivation of a living organoid biobank of colorectal cancer patients. Cell 161, 933-945. PMID: 25957691.
83) Fujii, M., Shimokawa, M., Date, S., Takano, A., Matano, M., Nanki, K., Ohta, Y., Toshimitsu, K., Nakazato, Y., Kawasaki, K., Uraoka, T., Watanabe, T., Kanai, T. and Sato, T. (2016). A colorectal tumor organoid library demonstrates progressive loss of niche factor requirements during tumorigenesis. Cell Stem Cell 18, 827-838.
84) Chua, C. W., Shibata, M., Lei, M., Toivanen, R., Barlow, L. J., Bergren, S. K., Badani, K. K., McKiernan, J. M., Benson, M. C., Hibshoosh, H. and Shen, M. M. (2014). Single luminal epithelial progenitors can generate prostate organoids in culture. Nat Cell Biol 16, 951-961. PMCID: PMC4183706.
85) Wang, Z. A., Toivanen, R., Bergren, S. K., Chambon, P. and Shen, M. M. (2014). Luminal cells are favored as the cell of origin for prostate cancer. Cell Rep 8, 1339-1346. PMCID: PMC4163115.
86) Wang, Z. A., Mitrofanova, A., Bergren, S. K., Abate-Shen, C., Cardiff, R. D., Califano, A. and Shen, M. M. (2013). Lineage analysis of basal epithelial cells reveals their unexpected plasticity and supports a cell-of-origin model for prostate cancer heterogeneity. Nat Cell Biol 15, 274-283. PMCID: PMC3743266.
87) Wang, X., Kruithof-de Julio, M., Economides, K. D., Walker, D., Yu, H., Halili, M. V., Hu, Y.-P., Price, S. M., Abate-Shen, C. and Shen, M. M. (2009). A luminal epithelial stem cell that is a cell of origin for prostate cancer. Nature 461, 495-500. PMCID: PMC2800362.
88) Puzio-Kuter, A. M., Castillo-Martin, M., Kinkade, C. W., Wang, X., Shen, T. H., Matos, T., Shen, M. M., Cordon-Cardo, C. and Abate-Shen, C. (2009). Inactivation of p53 and Pten promotes invasive bladder cancer. Genes Dev 23, 675-680. PMCID: PMC2661614.
89) Kobayashi, T., Wang, J., Al-Ahmadie, H. and Abate-Shen, C. (2013). ARF regulates the stability of p16 protein via REGgamma-dependent proteasome degradation. Mol Cancer Res 11, 828-833. PMCID: PMC3748223.
90) Ouyang, X., Jessen, W. J., Al-Ahmadie, H., Serio, A. M., Lin, Y., Shih, W. J., Reuter, V. E., Scardino, P. T., Shen, M. M., Aronow, B. J., Vickers, A. J., Gerald, W. L. and Abate-Shen, C. (2008). Activator protein-1 transcription factors are associated with progression and recurrence of prostate cancer. Cancer Res 68, 2132-2144. PMID: 18381418.
91) Al-Ahmadie, H., lyer, G., Hohl, M., Asthana, S., Inagaki, A., Schultz, N., Hanrahan, A. J., Scott, S. N., Brannon, A. R., McDermott, G. C., Pirun, M., Ostrovnaya, I., Kim, P., Socci, N. D., Viale, A., Schwartz, G. K., Reuter, V., Bochner, B. H., Rosenberg, J. E., Bajorin, D. F., Berger, M. F., Petrini, J. H., Solit, D. B. and Taylor, B. S. (2014). Synthetic lethality in ATM-deficient RAD50-mutant tumors underlies outlier response to cancer therapy. Cancer Discov 4, 1014-1021. PMCID: PMC4155059.
92) Aytes, A., Mitrofanova, A., Lefebvre, C., Alvarez, M. J., Castillo-Martin, M., Zheng, T., Eastham, J. A., Gopalan, A., Pienta, K. J., Shen, M. M., Califano, A. and Abate-Shen, C. (2014). Cross-species regulatory network analysis identifies a synergistic interaction between FOXM1 and CENPF that drives prostate cancer malignancy. Cancer Cell 25, 638-651. PMCID: PMC4051317.
93) Irshad, S., Bansal, M., Castillo-Martin, M., Zheng, T., Aytes, A., Wenske, S., Le Magnen, C., Guarnieri, P., Sumazin, P., Benson, M. C., Shen, M. M., Califano, A. and Abate-Shen, C. (2013). A molecular signature predictive of indolent prostate cancer. Sci Transl Med 5, 202ra122. PMCID: PMC3943244.
94) Kim, P. H., Cha, E. K., Sfakianos, J. P., lyer, G., Zabor, E. C., Scott, S. N., Ostrovnaya, I., Ramirez, R., Sun, A., Shah, R., Yee, A. M., Reuter, V. E., Bajorin, D. F., Rosenberg, J. E., Schultz, N., Berger, M. F., Al-Ahmadie, H. A., Solit, D. B. and Bochner, B. H. (2015). Genomic predictors of survival in patients with high-grade urothelial carcinoma of the bladder. Eur Urol 67, 198-201. PMCID: PMC4312739.
95) Cheng, D. T., Mitchell, T. N., Zehir, A., Shah, R. H., Benayed, R., Syed, A., Chandramohan, R., Liu, Z. Y., Won, H. H., Scott, S. N., Brannon, A. R., O'Reilly, C., Sadowska, J., Casanova, J., Yannes, A., Hechtman, J. F., Yao, J., Song, W., Ross, D. S., Oultache, A., Dogan, S., Borsu, L., Hameed, M., Nafa, K., Arcila, M. E., Ladanyi, M. and Berger, M. F. (2015). Memorial Sloan Kettering-Integrated Mutation Profiling of Actionable Cancer Targets (MSK-IMPACT): a hybridization capture-based next-generation sequencing clinical assay for solid tumor molecular oncology. J Mol Diagn 17, 251-264.
96) Jager, W., Moskalev, I., Janssen, C., Hayashi, T., Awrey, S., Gust, K. M., So, A. I., Zhang, K., Fazli, L., Li, E., Thuroff, J. W., Lange, D. and Black, P. C. (2013). Ultrasound-guided intramural inoculation of orthotopic bladder cancer xenografts: a novel high-precision approach. PLoS One 8, e59536. PMCID: PMC3608695.
97) Jager, W., Moskalev, I., Janssen, C., Hayashi, T., Gust, K. M., Awrey, S. and Black, P. C. (2014). Minimally invasive establishment of murine orthotopic bladder xenografts. J Vis Exp, e51123. PMCID: PMC4123502.
98) Grossman, H. B., Wedemeyer, G., Ren, L., Wilson, G. N. and Cox, B. (1986). Improved growth of human urothelial carcinoma cell cultures. J Urol 136, 953-959.
99) Guo, W., Keckesova, Z., Donaher, J. L., Shibue, T., Tischler, V., Reinhardt, F., Itzkovitz, S., Noske, A., Zurrer-Hardi, U., Bell, G., Tam, W. L., Mani, S. A., van Oudenaarden, A. and Weinberg, R. A. (2012). Slug and Sox9 cooperatively determine the mammary stem cell state. Cell 148, 1015-1028. PMCID: PMC3305806.
100) Lang, S. H., Stark, M., Collins, A., Paul, A. B., Stower, M. J. and Maitland, N. J. (2001). Experimental prostate epithelial morphogenesis in response to stroma and three-dimensional matrigel culture. Cell Growth Differ 12, 631-640. PMID: 11751458.
101) Cano, P., Godoy, A., Escamilla, R., Dhir, R. and Onate, S. A. (2007). Stromal-epithelial cell interactions and androgen receptor-coregulator recruitment is altered in the tissue microenvironment of prostate cancer. Cancer Res 67, 511-519. PMID: 17234758.
102) Liu, X., Ory, V., Chapman, S., Yuan, H., Albanese, C., Kallakury, B., Timofeeva, O. A., Nealon, C., Dakic, A., Simic, V., Haddad, B. R., Rhim, J. S., Dritschilo, A., Riegel, A., McBride, A. and Schlegel, R. (2012). ROCK inhibitor and feeder cells induce the conditional reprogramming of epithelial cells. Am J Pathol 180, 599-607. PMCID: PMC3349876.
103) Zhang, L., Valdez, J. M., Zhang, B., Wei, L., Chang, J. and Xin, L. (2011). ROCK inhibitor Y-27632 suppresses dissociation-induced apoptosis of murine prostate stem/progenitor cells and increases their cloning efficiency. PLoS ONE 6, e18271. PMCID: PMC3065488.
104) Xu, Y., Zhu, X., Hahm, H. S., Wei, W., Hao, E., Hayek, A. and Ding, S. (2010). Revealing a core signaling regulatory mechanism for pluripotent stem cell survival and self-renewal by small molecules. Proc Natl Acad Sci USA 107, 8129-8134. PMCID: PMC2889586.
105) Stange, D. E., Koo, B. K., Huch, M., Sibbel, G., Basak, O., Lyubimova, A., Kujala, P., Bartfeld, S., Koster, J., Geahlen, J. H., Peters, P. J., van Es, J. H., van de Wetering, M., Mills, J. C. and Clevers, H. (2013). Differentiated Troy+ chief cells act as reserve stem cells to generate all lineages of the stomach epithelium. Cell 155, 357-368. PMCID: PMC4094146.
106) Huch, M., Dorrell, C., Boj, S. F., van Es, J. H., Li, V. S., van de Wetering, M., Sato, T., Hamer, K., Sasaki, N., Finegold, M. J., Haft, A., Vries, R. G., Grompe, M. and Clevers, H. (2013). In vitro expansion of single Lgr5+ liver stem cells induced by Wnt-driven regeneration. Nature 494, 247-250. PMCID: PMC3634804.
107) Huch, M., Bonfanti, P., Boj, S. F., Sato, T., Loomans, C. J., van de Wetering, M., Sojoodi, M., Li, V. S., Schuijers, J., Gracanin, A., Ringnalda, F., Begthel, H., Hamer, K., Mulder, J., van Es, J. H., de Koning, E., Vries, R. G., Heimberg, H. and Clevers, H. (2013). Unlimited in vitro expansion of adult bi-potent pancreas progenitors through the Lgr5/R-spondin axis. Embo J 32, 2708-2721. PMCID: PMC3801438.
108) Sato, T., Vries, R. G., Snippert, H. J., van de Wetering, M., Barker, N., Stange, D. E., van Es, J. H., Abo, A., Kujala, P., Peters, P. J. and Clevers, H. (2009). Single Lgr5 stem cells build crypt-villus structures in vitro without a mesenchymal niche. Nature 459, 262-265.
109) Karthaus, W. R., laquinta, P. J., Drost, J., Gracanin, A., van Boxtel, R., Wongvipat, J., Dowling, C. M., Gao, D., Begthel, H., Sachs, N., Vries, R. G., Cuppen, E., Chen, Y., Sawyers, C. L. and Clevers, H. C. (2014). Identification of multipotent luminal progenitor cells in human prostate organoid cultures. Cell 159, 163-175. PMCID: PMC4772677.
110) Cerami, E., Gao, J., Dogrusoz, U., Gross, B. E., Sumer, S. O., Aksoy, B. A., Jacobsen, A., Byrne, C. J., Heuer, M. L., Larsson, E., Antipin, Y., Reva, B., Goldberg, A. P., Sander, C. and Schultz, N. (2012). The cBio cancer genomics portal: an open platform for exploring multidimensional cancer genomics data. Cancer Discov 2, 401-404. PMCID: PMC3956037.
111) Gao, J., Aksoy, B. A., Dogrusoz, U., Dresdner, G., Gross, B., Sumer, S. O., Sun, Y., Jacobsen, A., Sinha, R., Larsson, E., Cerami, E., Sander, C. and Schultz, N. (2013). Integrative analysis of complex cancer genomics and clinical profiles using the cBioPortal. Sci Signal 6, p11. PMCID: PMC4160307.
112) Bryan, R. T. (2015). Cell adhesion and urothelial bladder cancer: the role of cadherin switching and related phenomena. Philos Trans R Soc Lond B Biol Sci 370, 20140042. PMCID: PMC4275911.
113) Shen, R. and Seshan, V. E. (2016). FACETS: allele-specific copy number and clonal heterogeneity analysis tool for high-throughput DNA sequencing. Nucleic Acids Res 2016 June 7, Epub ahead of print. PMID: 27270079.
114) Chang, M. T., Asthana, S., Gao, S. P., Lee, B. H., Chapman, J. S., Kandoth, C., Gao, J., Socci, N. D., Solit, D. B., Olshen, A. B., Schultz, N. and Taylor, B. S. (2016). Identifying recurrent mutations in cancer reveals widespread lineage diversity and mutational specificity. Nat Biotechnol 34, 155-163. PMCID: PMC4744099.
115) Johnson, B. E., Mazor, T., Hong, C., Barnes, M., Aihara, K., McLean, C. Y., Fouse, S. D., Yamamoto, S., Ueda, H., Tatsuno, K., Asthana, S., Jalbert, L. E., Nelson, S. J., Bollen, A. W., Gustafson, W. C., Charron, E., Weiss, W. A., Smirnov, I. V., Song, J. S., Olshen, A. B., Cha, S., Zhao, Y., Moore, R. A., Mungall, A. J., Jones, S. J., Hirst, M., Marra, M. A., Saito, N., Aburatani, H., Mukasa, A., Berger, M. S., Chang, S. M., Taylor, B. S. and Costello, J. F. (2014). Mutational analysis reveals the origin and therapy-driven evolution of recurrent glioma. Science 343, 189-193. PMCID: PMC3998672.
116) Carter, S. L., Cibulskis, K., Heiman, E., McKenna, A., Shen, H., Zack, T., Laird, P. W., Onofrio, R. C., Winckler, W., Weir, B. A., Beroukhim, R., Pellman, D., Levine, D. A., Lander, E. S., Meyerson, M. and Getz, G. (2012). Absolute quantification of somatic DNA alterations in human cancer. Nat Biotechnol 30, 413-421. PMCID: PMC4383288.
117) Li, H. and Durbin, R. (2009). Fast and accurate short read alignment with Burrows-Wheeler transform. Bioinformatics 25, 1754-1760. PMCID: PMC2705234.
118) DePristo, M. A., Banks, E., Poplin, R., Garimella, K. V., Maguire, J. R., Hartl, C., Philippakis, A. A., del Angel, G., Rivas, M. A., Hanna, M., McKenna, A., Fennell, T. J., Kernytsky, A. M., Sivachenko, A. Y., Cibulskis, K., Gabriel, S. B., Altshuler, D. and Daly, M. J. (2011). A framework for variation discovery and genotyping using next-generation DNA sequencing data. Nat Genet 43, 491-498. PMCID: PMC3083463.
119) Cibulskis, K., Lawrence, M. S., Carter, S. L., Sivachenko, A., Jaffe, D., Sougnez, C., Gabriel, S., Meyerson, M., Lander, E. S. and Getz, G. (2013). Sensitive detection of somatic point mutations in impure and heterogeneous cancer samples. Nat Biotechnol 31, 213-219. PMCID: PMC3833702.
120) Ye, K., Schulz, M. H., Long, Q., Apweiler, R. and Ning, Z. (2009). Pindel: a pattern growth approach to detect break points of large deletions and medium sized insertions from paired-end short reads. Bioinformatics 25, 2865-2871. PMCID: PMC2781750.
121) Olshen, A. B., Venkatraman, E. S., Lucito, R. and Wigler, M. (2004). Circular binary segmentation for the analysis of array-based DNA copy number data. Biostatistics 5, 557-572. PMID: 15475419.
122) Venkatraman, E. S. and Olshen, A. B. (2007). A faster circular binary segmentation algorithm for the analysis of array CGH data. Bioinformatics 23, 657-663. PMID: 17234643.
123) Wang, J., Mullighan, C. G., Easton, J., Roberts, S., Heatley, S. L., Ma, J., Rusch, M. C., Chen, K., Harris, C. C., Ding, L., Holmfeldt, L., Payne-Turner, D., Fan, X., Wei, L., Zhao, D., Obenauer, J. C., Naeve, C., Mardis, E. R., Wilson, R. K., Downing, J. R. and Zhang, J. (2011). CREST maps somatic structural variation in cancer genomes with base-pair resolution. Nat Methods 8, 652-654. PMCID: PMC3527068.
124) Rausch, T., Zichner, T., Schlattl, A., Stutz, A. M., Banes, V. and Korbel, J. O. (2012). DELLY: structural variant discovery by integrated paired-end and split-read analysis. Bioinformatics 28, i333-i339. PMCID: PMC3436805.
125) Dobin, A., Davis, C. A., Schlesinger, F., Drenkow, J., Zaleski, C., Jha, S., Batut, P., Chaisson, M. and Gingeras, T. R. (2013). STAR: ultrafast universal RNA-seq aligner. Bioinformatics 29, 15-21. PMCID: PMC3530905.
126) Engstrom, P. G., Steijger, T., Sipos, B., Grant, G. R., Kahles, A., Ratsch, G., Goldman, N., Hubbard, T. J., Harrow, J., Guigo, R., Bertone, P. and Consortium, R. (2013). Systematic evaluation of spliced alignment programs for RNA-seq data. Nat Methods 10, 1185-1191. PMCID: PMC4018468.
127) Rapaport, F., Khanin, R., Liang, Y., Pirun, M., Krek, A., Zumbo, P., Mason, C. E., Socci, N. D. and Betel, D. (2013). Comprehensive evaluation of differential gene expression analysis methods for RNA-seq data. Genome Biol 14, R95. PMCID: PMC4054597.
128) Meacham, C. E. and Morrison, S. J. (2013). Tumour heterogeneity and cancer cell plasticity. Nature 501, 328-337. PMCID: PMC4521623.
129) Shibata, M. and Shen, M. M. (2013). The roots of cancer: stem cells and the basis for tumor heterogeneity. Bioassays 35, 253-260. PMCID: PMC3687804.
130) Greaves, M. and Maley, C. C. (2012). Clonal evolution in cancer. Nature 481, 306-313. PMCID: PMC3367003.
131) Marusyk, A., Tabassum, D. P., Altrock, P. M., Almendro, V., Michor, F. and Polyak, K. (2014). Non-cell-autonomous driving of tumour growth supports sub-clonal heterogeneity. Nature 514, 54-58. PMCID: PMC4184961.
132) Shin, K., Lim, A., Zhao, C., Sahoo, D., Pan, Y., Spiekerkoetter, E., Liao, J. C. and Beachy, P. A. (2014). Hedgehog signaling restrains bladder cancer progression by eliciting stromal production of urothelial differentiation factors. Cancer Cell 26, 521-533. PMCID: PMC4326077.
133) Kurtova, A. V., Xiao, J., Mo, Q., Pazhanisamy, S., Krasnow, R., Lerner, S. P., Chen, F., Roh, T. T., Lay, E., Ho, P. L. and Chan, K. S. (2015). Blocking PGE2-induced tumour repopulation abrogates bladder cancer chemoresistance. Nature 517, 209-213. PMCID: PMC4465385.
134) Eirew, P., Steif, A., Khattra, J., Ha, G., Yap, D., Farahani, H., Gelmon, K., Chia, S., Mar, C., Wan, A., Laks, E., Biele, J., Shumansky, K., Rosner, J., McPherson, A., Nielsen, C., Roth, A. J., Lefebvre, C., Bashashati, A., de Souza, C., Siu, C., Aniba, R., Brimhall, J., Oloumi, A., Osako, T., Bruna, A., Sandoval, J. L., Algara, T., Greenwood, W., Leung, K., Cheng, H., Xue, H., Wang, Y., Lin, D., Mungall, A. J., Moore, R., Zhao, Y., Lorette, J., Nguyen, L., Huntsman, D., Eaves, C. J., Hansen, C., Marra, M. A., Caldas, C., Shah, S. P. and Aparicio, S. (2015). Dynamics of genomic clones in breast cancer patient xenografts at single-cell resolution. Nature 518, 422-426. PMCID: PMC4864027.
135) Kreso, A., O'Brien, C. A., van Galen, P., Gan, O. I., Notta, F., Brown, A. M., Ng, K., Ma, J., Wienholds, E., Dunant, C., Pollett, A., Gallinger, S., McPherson, J., Mullighan, C. G., Shibata, D. and Dick, J. E. (2013). Variable clonal repopulation dynamics influence chemotherapy response in colorectal cancer. Science 339, 543-548. PMID: 23239622.
136) Misale, S., Yaeger, R., Hobor, S., Scala, E., Janakiraman, M., Liska, D., Valtorta, E., Schiavo, R., Buscarino, M., Siravegna, G., Bencardino, K., Cercek, A., Chen, C. T., Veronese, S., Zanon, C., Sartore-Bianchi, A., Gambacorta, M., Gallicchio, M., Vakiani, E., Boscaro, V., Medico, E., Weiser, M., Siena, S., Di Nicolantonio, F., Solit, D. and Bardelli, A. (2012). Emergence of KRAS mutations and acquired resistance to anti-EGFR therapy in colorectal cancer. Nature 486, 532-536. PMCID: PMC3927413.
137) Poulikakos, P. I., Persaud, Y., Janakiraman, M., Kong, X., Ng, C., Moriceau, G., Shi, H., Atefi, M., Titz, B., Gabay, M. T., Salton, M., Dahlman, K. B., Tadi, M., Wargo, J. A., Flaherty, K. T., Kelley, M. C., Misteli, T., Chapman, P. B., Sosman, J. A., Graeber, T. G., Ribas, A., Lo, R. S., Rosen, N. and Solit, D. B. (2011). RAF inhibitor resistance is mediated by dimerization of aberrantly spliced BRAF(V600E). Nature 480, 387-390. PMCID: PMC3266695.
138) Solit, D. B., Zheng, F. F., Drobnjak, M., Munster, P. N., Higgins, B., Verbal, D., Heller, G., Tong, W., Cordon-Cardo, C., Agus, D. B., Scher, H. I. and Rosen, N. (2002). 17-Allylamino-17-demethoxygeldanamycin induces the degradation of androgen receptor and HER-2/neu and inhibits the growth of prostate cancer xenografts. Clin Cancer Res 8, 986-993. PMID: 12006510.
139) Solit, D. B., Basso, A. D., Olshen, A. B., Scher, H. I. and Rosen, N. (2003). Inhibition of heat shock protein 90 function down-regulates Akt kinase and sensitizes tumors to Taxol. Cancer Res 63, 2139-2144. PMID: 12727831.
140) Solit, D. B., Garraway, L. A., Pratilas, C. A., Sawai, A., Getz, G., Basso, A., Ye, Q., Lobo, J. M., She, Y., Osman, I., Golub, T. R., Sebolt-Leopold, J., Sellers, W. R. and Rosen, N. (2006). BRAF mutation predicts sensitivity to MEK inhibition. Nature 439, 358-362. PMCID: PMC3306236.
141) She, Q. B., Halilovic, E., Ye, Q., Zhen, W., Shirasawa, S., Sasazuki, T., Solit, D. B. and Rosen, N. (2010). 4E-BP1 is a key effector of the oncogenic activation of the AKT and ERK signaling pathways that integrates their function in tumors. Cancer Cell 18, 39-51. PMCID: PMC3286650.

### Example 8: Methods for Bladder Cancer Organoid Culture

In one embodiment, the bladder organoids of the invention can be generated using the following protocol:

1. Prepare ice cold Gentamycin (4 mg/ml in PBS) in a culture dish.

2. Transfer the patient tissues into the dish filled with Gentamycin (4 mg/ml) and incubate for 5 min at RT.

3. Wash the Gentamycin-treated patient tissues in cold PBS.

4. Fill the e-tube with 600 ul of pre-warmed 1× collagenase/hyaluronidase sol (1/10) and transfer the Gentamycin-treated patient tissues into the e-tube.

5. Using small, sharp sterile scissors, macerate the tissues to cut into small pieces.

6. Fill the 50 ml tube with 10 ml of pre-warmed 1× collagenase/hyaluronidase sol. and transfer the small pieces of patient samples into the tube.

7. Incubate the tissues with 1× collagenase/hyaluronidase in 37 C incubator for 10 min.

8. Dissociate the tissues by pipetting with 1 ml pipette tip.

9. Centrifuge at 350 rcf for 5 min and discard supernatant.

10. Add 10 ml of HBSS (2% FBS) into the tubes and filter through a 100 uM cell strainer.

11. Centrifuge at 350 rcf for 5 min and discard supernatant.

12. Resuspend the pellets with 60% Matrigel and plate 250 ul of Matrigel/cell mixture at the center of the well in the pre-coated 6-well plate.

13. Incubate the 6-well plate in 37 C incubator for 30 min.

14. Add 1.5 ml of pre-warmed organoid culture media to each well.

In another embodiment, the bladder organoids of the invention can be generated using the following protocol:

1. Prepare ice cold Gentamycin (4 mg/ml in PBS) in a culture dish.

2. Transfer the patient tissues into the dish filled with Gentamycin (4 mg/ml) and incubate for 5 min at RT.

3. Wash the Gentamycin-treated patient tissues in cold PBS.

4. Fill the e-tube with 600 ul of pre-warmed 1× collagenase/hyaluronidase sol (1/10) and transfer the Gentamycin-treated patient tissues into the e-tube.

5. Using small, sharp sterile scissors, macerate the tissues to cut into small pieces.

6. Fill the 50 ml tube with 10 ml of pre-warmed 1× collagenase/hyaluronidase sol. and transfer the small pieces of patient samples into the tube.

7. Incubate the tissues with 1× collagenase/hyaluronidase in 37 C incubator for 10 min.

8. Centrifuge at 350 rcf for 5 min and discard supernatant.

9. Add 2.5 ml of PBS and 2.5 ml of TrypLE to the pellets and resuspend the pellets with 1 ml pipette tip. Incubate at RT for 3 min.

10. Add 10 ml of HBSS (2% FBS).

11. Centrifuge at 350 rcf for 5 min and discard supernatant.

12. Prepare the pre-warmed DNaseI solution (final 1 mg/ml)

13. Resuspend the pellets in 2 ml of DNaseI solution with 1 ml pipette tip.

14. Incubate tissues with DNaseI for 5 min at RT.

15. Add 10 ml of HBSS (2% FBS) into the tubes and filter through a 70 uM cell strainer.

16. Centrifuge at 350 rcf for 5 min and discard supernatant.

17. Resuspend pellet with 60% Matrigel and plate 250 ul of Matrigel/cell mixture at the center of the well in the pre-coated 6-well plate.

18. Incubate the 6-well plate in 37 C incubator for 30 min.

19. Add 1.5 ml of pre-warmed organoid culture media to each well.

1. A method for culturing a bladder cell line, the method comprising:
a) obtaining a sample of bladder tissue from a subject;
b) dissociating the sample of bladder tissue;
c) isolating dissociated bladder epithelial cells from the sample of bladder tissue;
d) plating the isolated dissociated bladder epithelial cells of (c) on an adherent cell culture support; and
e) culturing the dissociated bladder epithelial cells in a culture medium comprising hepatocyte medium, FBS, Matrigel, and ROCK inhibitor;
wherein the dissociated bladder epithelial cells form bladder cell line colonies in culture.

2. A method for culturing a bladder organoid, the method comprising:
a) obtaining a sample of bladder tissue from a subject;
b) dissociating the sample of bladder tissue;
c) isolating dissociated bladder epithelial cells from the sample of bladder tissue;
d) plating the isolated dissociated bladder epithelial cells of (c) on a low attachment cell culture support; and
e) culturing the dissociated bladder epithelial cells in a culture medium comprising hepatocyte medium, FBS, Matrigel, and ROCK inhibitor;
wherein the dissociated bladder epithelial cells form organoids in culture.

3. A method for culturing a bladder organoid, the method comprising:
a) obtaining a sample of bladder tissue from a subject;
b) dissociating the sample of bladder tissue;
c) contacting the dissociated bladder tissue with a Matrigel solution and plating in a cell culture support, wherein the Matrigel solution comprises hepatocyte medium and Matrigel and wherein the Matrigel solution forms a matrix;
d) providing an overlay layer of liquid culture medium comprising hepatocyte medium and FBS; and
e) incubating the culture of (d) wherein the dissociated bladder tissue forms organoids.

4. (canceled)

5. (canceled)

6. (canceled)

7. The method of claim 3, wherein the bladder tissue is cancerous or obtained from a bladder tumor.

8. (canceled)

9. The method of claim 3, wherein the subject is a human.

10. The method of claim 3, wherein the bladder tissue is obtained from an endoscopic biopsy, an endoscopic resection, or a cystectomy sample.

11. (canceled)

12. (canceled)

13. The method of claim 7, wherein the bladder organoid displays the transformed phenotype of the cancerous bladder tissue or bladder tumor.

14. (canceled)

15. The method of claim 3, wherein the culture medium further comprises Glutamax, EGF, antibiotic-antimycotic, 5% heat-inactivated charcoal-stripped FBS or a combination thereof.

16. (canceled)

17. (canceled)

18. The method of claim 15, wherein the culture medium comprises 10 ng/ml of EGF.

19. (canceled)

20. (canceled)

21. (canceled)

22. (canceled)

23. The method of claim 2, wherein an adherent bladder cell line is obtained from the organoids.

24. The method of claim 3, wherein an adherent bladder cell line is obtained from the organoids.

25. (canceled)

26. The method of claim 3, wherein a single cell suspension is obtained by the dissociating of (b).

27. The method of claim 3, wherein cell clusters are obtained by the dissociating of (b).

28. The method of claim 26, wherein the single cell suspension contains epithelial and stromal cells.

29. The method of claim 27, wherein the cell clusters contain epithelial and stromal cells.

30. (canceled)

31. The method of claim 3, wherein (b) comprises dissociating the sample of bladder tissue with collagenase, hyaluronidase, or a combination thereof.

32. The method of claim 31, wherein the dissociating further comprises dissociating the sample with TrypLE™ or trypsin.

33. (canceled)

34. (canceled)

35. (canceled)

36. (canceled)

37. The method of claim 3, further comprising:
f) serially passaging the organoids.

38. (canceled)

39. (canceled)

40. (canceled)

41. (canceled)

42. The method of claim 3, wherein the cell culture support is a 6-well tissue culture plate.

43. The method of claim 3, wherein the cell culture support is surface modified before the plating by rinsing Matrigel solution over the support surface and incubating the cell culture support at 37° C. for at least 30 minutes.

44. (canceled)

45. (canceled)

46. A bladder cell line, wherein the cell line is obtained by the method of claim 24.

47. (canceled)

48. (canceled)

49. (canceled)

50. (canceled)

51. (canceled)

52. A bladder organoid, wherein the organoid is obtained by the method of claim 3.

53. (canceled)

54. The cell line of claim 46, wherein the bladder cell line is a bladder tumor cell line and displays the transformed phenotype of cancerous bladder tissue.

55. The organoid of claim 52, wherein the bladder organoid is a bladder tumor organoid and displays the transformed phenotype of cancerous bladder tissue.

56. (canceled)

57. (canceled)

58. (canceled)

59. (canceled)

60. (canceled)

61. (canceled)

62. (canceled)

63. A method for treating bladder cancer in a subject in need thereof, comprising:
a) obtaining a sample of bladder tissue from the subject;
b) dissociating the sample of bladder tissue;
c) isolating dissociated bladder epithelial cells from the sample of bladder tissue;
d) plating the isolated dissociated bladder epithelial cells of (c) on an adherent cell culture support;
e) culturing the dissociated bladder epithelial cells in a culture medium comprising hepatocyte medium, FBS, Matrigel, and ROCK inhibitor, wherein the dissociated bladder epithelial cells form bladder cell line colonies in culture;
f) contacting the bladder cell line with a test compound; and
g) determining whether growth of the bladder cell line is inhibited in the presence of the test compound, as compared to growth of the bladder cell line in the absence of the test compound, wherein (i) the test compound is administered to the subject if growth of the bladder cell line is inhibited in the presence of the test compound; or wherein (ii) a cystectomy is performed on the subject if growth of the bladder cell line is not inhibited in the presence of the test compound.

64. (canceled)

65. The method of claim 63, wherein the test compound is an intravesical agent, an antineoplastic agent, or a chemotherapy agent.

66. (canceled)

67. (canceled)

68. The method of claim 63, wherein the growth of the bladder cell line of (f) is measured using a MTT assay.

69. A method for treating bladder cancer in a subject in need thereof, comprising:
a) obtaining a sample of bladder tissue from the subject;
b) dissociating the sample of bladder tissue;
c) contacting the dissociated bladder tissue with a Matrigel solution and plating in a cell culture support, wherein the Matrigel solution comprises hepatocyte medium and Matrigel and wherein the Matrigel solution forms a matrix;
d) providing an overlay layer of liquid culture medium comprising hepatocyte medium and FBS;
e) incubating the culture of (d) wherein the dissociated bladder tissue forms organoids;
f) contacting the bladder organoid with a test compound; and
g) determining whether growth of the bladder organoid is inhibited in the presence of the test compound, as compared to growth of the bladder organoid in the absence of the test compound, wherein (i) the test compound is administered to the subject if growth of the bladder organoid is inhibited in the presence of the test compound, or wherein (ii) a cystectomy is performed on the subject if growth of the bladder organoid is not inhibited in the presence of the test compound.

70. (canceled)

71. The method of claim 69, wherein the test compound is an intravesical agent, an antineoplastic agent, or a chemotherapy agent.

72. (canceled)

73. (canceled)

74. The method of claim 69, wherein the growth of the bladder organoid of (f) is measured using a MTT assay.

75. The method of claim 3, wherein the method has at least 80% efficiency.

76. (canceled)

77. (canceled)

78. (canceled)
